SEQUENCE LISTING (9 sequences)

SEQ ID NO: 1 - DNA, 5985 bp, homo sapiens
  CDS (1)..(5985)
atggacacttctcgcacgccgagtgtgtgcagggaaacgggcggagcagccctgagcagaggtctggctaacacctcctacaccagcccaggcctccagaggctgaaggactctccacaggacaggatggtggggacacagggctgtgtgagcactgctctctctgtagccccggacaaaggccagtgctccacgtggggggctggtcacttctccaccttcgaccaccacgtgtacgacttctcggggacgtgcaactacatcttcgcggccacctgcaaggacgccttccccaccttcagtgtccagctgcggcgaggcccagacgggagcatctcgcggatcatcgtggagctgggggcctccgtcgtcactgtgagcgaagccatcatctcagtcaaggacatcggggtcatcagcctgccctataccagcaatggactccagatcacacccttcggccagagcgtgcggctggtggccaagcagctggagctggagctggaagtcgtgtggggtcctgacagccacctcatggttctggtggagcggaagtacatgggtcagatgtgcgggctctgcgggaactttgacgggaaggtgaccaacgagtttgtcagtgaggagggtacggtcctgaatgacctctccaataaccacacctgcgtgcccgtcacccagtgcccctgtgtgctccacggcgccatgtatgcccccggggaggtcacaatagctgcctgccaaacctgccggtgcaccctgggccgctgggtgtgcacggagcggccgtgccccggacactgctccctggaaggtggctcctttgttaccacatttgacgccaggccctaccgcttccacggcacctgcacctacatcctcctccagagcccccagcttcccgaggacggtgccctcatggctgtgtacgacaagtccggcgtctcacactccgagacctccctggtggctgtggtctacctctccaggcaggacaaaattgtgatctctcaggacgaggtggtcaccaacaacggagaagccaagtggctgccatacaagactcgcaacatcacggtcttcaggcagacgtccacccacctccagatggccaccagcttcgggctggagctcgtggtccagctgcgccccatcttccaggcctatgtcactgttgggccccagttcagaggtcagaccagagggctctgcggcaacttcaacggggacacaacggatgacttcaccactagcatgggtatcgccgagggcaccgcctcgctgtttgtggactcctggcgggcggggaactgtccggccgctctggagcgtgagactgacccctgctccatgagccagctcaacaaggtgtgtgcagagacccactgctccatgctgctgaggacaggcacggtgttcgagaggtgccacgccacagtgaaccctgcacccttctacaagaggtgcgtgtaccaggcctgcaactacgaggagacctttccccacatctgtgccgccctgggcgactacgtacacgcctgctccttgcggggcgtcctgctctggggctggagaagcagtgtggacaactgcaccatcccctgcacgggtaacaccaccttcagctacaacagccaagcctgtgagcgcacctgcctgtcgctgtcggaccgtgccaccgagtgccaccacagcgccgtgcccgtggacggttgcaactgccccgatggcacctacctgaaccaaaagggcgagtgtgtgcgcaaggcccagtgcccgtgcatactggagggttacaagttcatcctggccgagcagtccactgtcatcaacggcatcacctgccactgcatcaacgggcggctgagttgcccgcagcggccacagatgttcctggcctcctgccaggcccctaagaccttcaagtcctgcagccagtcctccgagaacaagtttggggcagcctgtgcccccacatgccagatgctggccaccggtgttgcctgcgtgcccaccaagtgtgagcctggctgtgtctgcgccgagggcctctacgagaatgccgacgggcagtgtgtgccccccgaggagtgcccatgtgagttctcggggggccacgtcatcaccttcgacggccagcgcttcgtattcgacggcaactgcgagtacatcctggccacggtaaccatcgggcgccaggccgcaggggccggggacccagaggcacggcctccagggctcctcctcagcgccctctccctgcaggacgtctgtggtgtcaacgactcacagcccaccttcaagatcctgacagagaacgtcatctgtgggaactccggggtcacatgctcacgggccatcaagatcttcctggggggcctgtccgtggtgctggcggacagaaactacacggtcaccggggaggagccccacgtgcagctcggggtgacgccgggtgcgctgagccttgtcgtggacatcagcatccccgggaggtacaacctgacgctcatctggaacaggcacatgaccatcctcatcaggatcgcccgtgcctcccaggatcccctctgcggcttgtgtggcaacttcaacgggaacatgaaggacgacttcgagacgcgcagcaggtacgtggcatccagcgagctggagttggtgaactcgtggaaggagagcccgctgtgcggggacgtgagcttcgtgacagacccctgcagtctcaatgccttccggcgctcctgggccgagcgcaagtgcagcgtcatcaacagccagacctttgccacctgccacagcaagccagacactgactggcacacacccctgcaggtataccacctgccctactacgaggcctgcgtgcgcgacgcatgtgggtgtgacagtggcggggactgtgagtgtctgtgcgatgccgtggctgcctacgcccaagcctgtctggacaagggtgtgtgcgtggactggaggaccccggccttctgccccatctactgcggcttctacaacacgcacacgcaggacggccatggcgagtaccagtacacacaggaggccaactgcacgtggcactaccagccctgcctctgccccagccagccacagagcgtcccaggcagcaacatcgaaggctgctacaactgctcccaggatgagtacttcgaccacgaggagggggtgtgcgtgccctgcagctcacggcccacgcaagtctggcccatgacgggaacctccaccaccatcgggcttctcagctccaccggaccctcacccagctctaatcacacccctgccagccccacccagacacccctccttccagccacgctcacatcctccaagcccacagcctcctcgggaggtaaggagcctccagctgagcccatggagagggcagctgcaggaggtcctagccacactgagatcgacagccacaaaacccacagtgacccaggccacaaccagggccacggcatcgaccgccagcccagccacgacgtccacagctcagtccacaacacggaccacaatgacactaccaaccccagccacatcagggacaagccccacgctgctactcacacagtcatcacccctacccacgcacagatggccacatctgcctccatccactcagcgccaacaggtaccattcctccaccaacaacgctcaaggccacagggtccacccacacagccccaccaataacgccgaccaccagtgggaccagccaagcccacagctcattcagcacaaacaaaacacctacctcgctacattcacacacttcctccacacaccatcctgaagtcaccccaacttctactaccacgattactcccaaccccaccagtacaggcaccagaacccctgtggcccacaccacctcggccaccagcagcagactacccacacccttcaccacacattccccacctacagggagcagtcccatctcttccacaggtcctatgactgcaccatcctttcatgccaccactacctatccaaccccatcacaccctcagaccacacttcccactcacgttccatctttctccacctccttggtgactccaagtactcacatagtcatcacccctacccacgcacagatggccacttctgcctccatccactcaatgcaaacaggcaccattcctccaccgaccacgatcaaggccacagggtccacccacacagccccaccaatgacaccgaccaccagtgggaccagccaatccctaagctcatttagcacggccaaaacttctacatccctaccttaccacacttcctccacacaccatcctgaagtcaccccaacttctaccaccaacatcacccccaaacacaccagtacaggcaccagaacccctgtggcccacaccacctcggccaccagcagcagactacccacacccttcaccacacattccccacctacagggagcagtcccatctcttccacagaccaccactacctatccaaccccatcacaccctcagaccacacttcccactcacgttccacctttctccacctccttggtgactccaagtactcacaaggtcatcacccctacccatgcacagatggccacttctgcctccatccactcaacgccaacagggcaccattccttccactgacaacgctcatgaacacagggtccacacacacagccccactaataacagtgaccaccagtaggaccagccaagtccacagctccttcagcacagccaaaacctctacatccctcctctcccatgcttcctccacacaccatccagaaatcaccacaaattctaccaccaccattactcccaaccccactagtacaggcaccggaacccctgtggcccacaccacctcagccaccagcagcaggctaaccaccacccttcaccacacactccccacctacagagagcagtcccttctcttccacaggtcctatgactgcaacatccttccagaccaccactacctatccaaccccatcacaccctcagaccacacttcccactcacgttccacctttctccacctctttagtgactccaagtactcacacagtcatcacccctacccatgcacagatgtccacttctgcctcgatccactcaatgccaacagtcaccaaccttaccaccaggcaccctggtcccaccttgtcgcctaccacacggttcctgaccagctccctcactgcccatggaagcacccctgcttctgccccggtatcttctctcgggacacctacgcccacctcacccgcaaccacccagggactgggtccctcgcatgcatcgacctccaccaggcgacaacgccacagttgccttcgtggtctctcacgtgggtggcagctcgttgctgcaagctgagggaatcttggttcgggtccctccctgagaccgggacttgggtgcaaggtgtaaccagggaggtgaccccaagaagcagaggcgagggagcaggaaccagctgggaggggagggcagctggggaaggcagggcctatggaagcacccagagtcctgaccctcccggagaaagccctctgcagcgggcagctggggcacacggagctcctgcaacaccatatgtcccgctctggggtcactggcacggtgtcctcggcccccctgcaggtcctgggtctggccaaccagagaggcccatgcccacaggggtctgcagtgtgcgggagcagcaggaggagatcacgttcaaggggtgcatggcgaacgtgacggtaacccgctgtgagggcgcctgcatttccgctgccagcttcaacatcatcacccagcaggtggatgcccgctgcagctgctgccgccccctccactcctatgagcagcagctggagctgccctgccccgatcccagcacgcctggccggcggctcgtactcaccctgcaggtgttcagccactgcgtgtgcagctctgtggcctgtggagactag

SEQ ID NO: 2 - protein, 1994 aa, homo sapiens
MDTSRTPSVCRETGGAALSRGLANTSYTSPGLQRLKDSPQDRMVGTQGCVSTALSVAPDKGQCSTWGAGHFSTFDHHVYDFSGTCNYIFAATCKDAFPTFSVQLRRGPDGSISRIIVELGASVVTVSEAIISVKDIGVISLPYTSNGLQITPFGQSVRLVAKQLELELEVVWGPDSHLMVLVERKYMGQMCGLCGNFDGKVTNEFVSEEGTVLNDLSNNHTCVPVTQCPCVLHGAMYAPGEVTIAACQTCRCTLGRWVCTERPCPGHCSLEGGSFVTTFDARPYRFHGTCTYILLQSPQLPEDGALMAVYDKSGVSHSETSLVAVVYLSRQDKIVISQDEVVTNNGEAKWLPYKTRNITVFRQTSTHLQMATSFGLELVVQLRPIFQAYVTVGPQFRGQTRGLCGNFNGDTTDDFTTSMGIAEGTASLFVDSWRAGNCPAALERETDPCSMSQLNKVCAETHCSMLLRTGTVFERCHATVNPAPFYKRCVYQACNYEETFPHICAALGDYVHACSLRGVLLWGWRSSVDNCTIPCTGNTTFSYNSQACERTCLSLSDRATECHHSAVPVDGCNCPDGTYLNQKGECVRKAQCPCILEGYKFILAEQSTVINGITCHCINGRLSCPQRPQMFLASCQAPKTFKSCSQSSENKFGAACAPTCQMLATGVACVPTKCEPGCVCAEGLYENADGQCVPPEECPCEFSGGHVITFDGQRFVFDGNCEYILATVTIGRQAAGAGDPEARPPGLLLSALSLQDVCGVNDSQPTFKILTENVICGNSGVTCSRAIKIFLGGLSVVLADRNYTVTGEEPHVQLGVTPGALSLVVDISIPGRYNLTLIWNRHMTILIRIARASQDPLCGLCGNFNGNMKDDFETRSRYVASSELELVNSWKESPLCGDVSFVTDPCSLNAFRRSWAERKCSVINSQTFATCHSKPDTDWHTPLQVYHLPYYEACVRDACGCDSGGDCECLCDAVAAYAQACLDKGVCVDWRTPAFCPIYCGFYNTHTQDGHGEYQYTQEANCTWHYQPCLCPSQPQSVPGSNIEGCYNCSQDEYFDHEEGVCVPCSSRPTQVWPMTGTSTTIGLLSSTGPSPSSNHTPASPTQTPLLPATLTSSKPTASSGGKEPPAEPMERAAAGGPSHTEIDSHKTHSDPGHNQGHGIDRQPSHDVHSSVHNTDHNDTTNPSHIRDKPHAATHTVITPTHAQMATSASIHSAPTGTIPPPTTLKATGSTHTAPPITPTTSGTSQAHSSFSTNKTPTSLHSHTSSTHHPEVTPTSTTTITPNPTSTGTRTPVAHTTSATSSRLPTPFTTHSPPTGSSPISSTGPMTAPSFHATTTYPTPSHPQTTLPTHVPSFSTSLVTPSTHIVITPTHAQMATSASIHSMQTGTIPPPTTIKATGSTHTAPPMTPTTSGTSQSLSSFSTAKTSTSLPYHTSSTHHPEVTPTSTTNITPKHTSTGTRTPVAHTTSATSSRLPTPFTTHSPPTGSSPISSTDHHYLSNPITPSDHTSHSRSTFLHLLGDSKYSQGHHPYPCTDGHFCLHPLNANRAPFLPLTTLMNTGSTHTAPLITVTTSRTSQVHSSFSTAKTSTSLLSHASSTHHPEITTNSTTTITPNPTSTGTGTPVAHTTSATSSRLTTTLHHTLPTYREQSLLFHRSYDCNILPDHHYLSNPITPSDHTSHSRSTFLHLFSDSKYSHSHHPYPCTDVHFCLDPLNANSHQPYHQAPWSHLVAYHTVPDQLPHCPWKHPCFCPGIFSRDTYAHLTRNHPGTGSLACIDLHQATTPQLPSWSLTWVAARCCKLRESWFGSLPETGTWVQGVTREVTPRSRGEGAGTSWEGRAAGEGRAYGSTQSPDPPGESPLQRAAGAHGAPATPYVPLWGHWHGVLGPPAGPGSGQPERPMPTGVCSVREQQEEITFKGCMANVTVTRCEGACISAASFNIITQQVDARCSCCRPLHSYEQQLELPCPDPSTPGRRLVLTLQVFSHCVCSSVACGD

SEQ ID NO: 3 - protein, 2258 aa, homo sapiens
  mat_peptide (19)..(2258)
MVQRWLLLSCCGALLSAGLANTSYTSPGLQRLKDSPQTAPDKGQCSTWGAGHFSTFDHHVYDFSGTCNYIFAATCKDAFPSFSVQLRRGPDGSISRIIVELGASVVTVSEAIISVKDIGVISLPYTSNGLQITPFGQSVRLVAKQLELELEVVWGPDSHLMVLVERKYMGQMCGLCGNFDGKVTNEFVSEEGKFLEPHKFAALQKLDDPGEICTFQDIPSTHVRQAQHARGCTQLLTLVAPECSVSKEPFVLSCQADVAAAPQPGPQNSSYATLSEYSRQCSMVGQPVALRSPGLCSVGQCPANQVYQECGSACVKTCSNSEHSCSSSCTFGCFCPEGTDLNDLSNNHTCVPVTQCPCVLHGAMYAPGEVTIAACQTCRCTLGRWVCTERPCPGHCSLEGGSFVTTFDARPYRFHGTCTYILLQSPQLPEDGALMAVYDKSGVSHSETSLVAVVYLSRQDKIVISQDEVVTNNGEAKWLPYKTRNITVFRQTSTHLQMATSFGLELVVQLRPIFQAYVTVGPQFRGQTRGLCGNFNGDTTDDFTTSMGIAEGTASLFVDSWRAGNCPDALERETDPCSMSQLNKVCAETHCSMLLRTGTVFERCHATVNPAPIYKRCMYQACNYEETFPHICAALGDYVHACSLRGVLLWGWRSSVDNCTIPCTGNTTFSYNSQACERTCLSLSDRATECHHSAVPVDGCNCPDGTYLNQKGECVRKAQCPCILEGYKFILAEQSTVINGITCHCINGRLSCPQRLQMFLASCQAPKTFKSCSQSSENKFGAACAPTCQMLATGVACVPTKCEPGCVCAEGLYENAYGQCVPPEECPCEFSGVSYPGGAELHTDCRTCSCSRGRWACQQGTHCPSTCTLYGEGHVITFDGQRFVFDGNCEYILATDVCGVNYSQPTFKILTENVICGNSGVTCSRAIKIFLGGLSVVLADRNYTVTGEEPHVQLGVTPGALSLVVDISIPGRYNLTLIWNRHMTILIRIARASQDPLCGLCGNFNGNMKDDFETRSRYVASSELELVNSWKESPLCGDVSFVTDPCSLNAFRRSWAERKCSVINSQTFATCHSKVYHLPYYEACVRDACGCDSGGDCECLCDAVAAYAQACLDKGVCVDWRTPAFCPIYCGFYNTHTQDGHGEYQYTQEANCTWHYQPCLCPSQPQSVPGSNIEGCYNCSQDEYFDHEEGVCVPCMPPTTPQPPTTPQLPTTGSRPTQVWPMTGTSTTIGLLSSTGPSPSSNHTPASPTQTPLLPATLTSSKPTASSGEPPRPTTAVTPQATSGLPPTATLRSTATKPTVTQATTRATASTASPATTSTAQSTTRTTMTLPTPATSGTSPTLPKSTNQELPGTTATQTTGPRPTPASTTGPTTPQPGQPTRPTATETTQTRTTTEYTTPQTPHTTHSPPTAGSPVPSTGPVTATSFHATTTYPTPSHPETTLPTHVPPFSTSLVTPSTHTVITPTHAQMASSASNHSAPTGTIPPPTTLKATGSTHTAPPITPTTSGTSQAHSSFSTNKTPTSLHSHTSSTHHPEVTPTSTTSITPNPTSTRTRTPMAHTNSATSSRPPTPFTTHSPPTGSSPISSTGPMTAPSFHATTTYPTPSHPQTTLPTHVPSFSTSLVTPSTHIVITPTHAQMATSASIHSMQTGTIPPPTTIKATGSTHTAPPMTPTTSGTSQSLSSFSTAKTSTSLPYHTSSTHHPEVTPTSTTNITPKHTSTGTRTPVAHTTSATSSRLPTPFTTHSPPTGSSPISSTDHHYLSNPITPSDHTSHSRSTFLHLLGDSKYSQGHHPYPCTDGHFCLHPLNANRAPFLPLTTLMNTGSTHTAPLITVTTSRTSQVHSSFSTAKTSTSLLSHASSTHHPEITTNSTTTITPNPTSTGTGTPVAHTTSATSSRLTTTLHHTLPTYREQSLLFHRSYDCNILPDHHYLSNPITPSDHTSHSRSTFLHLFSDSKYSHSHHPYPCTDVHFCLDPLNANSHQPYHQAPWSHLVAYHTVPDQLPHCPWKHPCFCPGIFSRDTYAHLTRNHPGTGSLACIDLHQATTPQLPSWSLTWVAARCCKLRESWFGSLPETGTWVQGVTREVTPRSRGEGAGTSWEGRAAGEGRAYGSTQSPDPPGESPLQRAAGAHGAPATPYVPLWGHWHGVLGPPAGPGSGQPERPMPTGVCSVREQQEEITFKGCMANVTVTRCEGACISAASFNIITQQVDARCSCCRPLHSYEQQLELPCPDPSTPGRRLVLTLQVFSHCVCSSVACGD

SEQ ID NO: 4 - protein, 2240 aa, homo sapiens
LANTSYTSPGLQRLKDSPQTAPDKGQCSTWGAGHFSTFDHHVYDFSGTCNYIFAATCKDAFPSFSVQLRRGPDGSISRIIVELGASVVTVSEAIISVKDIGVISLPYTSNGLQITPFGQSVRLVAKQLELELEVVWGPDSHLMVLVERKYMGQMCGLCGNFDGKVTNEFVSEEGKFLEPHKFAALQKLDDPGEICTFQDIPSTHVRQAQHARGCTQLLTLVAPECSVSKEPFVLSCQADVAAAPQPGPQNSSYATLSEYSRQCSMVGQPVALRSPGLCSVGQCPANQVYQECGSACVKTCSNSEHSCSSSCTFGCFCPEGTDLNDLSNNHTCVPVTQCPCVLHGAMYAPGEVTIAACQTCRCTLGRWVCTERPCPGHCSLEGGSFVTTFDARPYRFHGTCTYILLQSPQLPEDGALMAVYDKSGVSHSETSLVAVVYLSRQDKIVISQDEVVTNNGEAKWLPYKTRNITVFRQTSTHLQMATSFGLELVVQLRPIFQAYVTVGPQFRGQTRGLCGNFNGDTTDDFTTSMGIAEGTASLFVDSWRAGNCPDALERETDPCSMSQLNKVCAETHCSMLLRTGTVFERCHATVNPAPIYKRCMYQACNYEETFPHICAALGDYVHACSLRGVLLWGWRSSVDNCTIPCTGNTTFSYNSQACERTCLSLSDRATECHHSAVPVDGCNCPDGTYLNQKGECVRKAQCPCILEGYKFILAEQSTVINGITCHCINGRLSCPQRLQMFLASCQAPKTFKSCSQSSENKFGAACAPTCQMLATGVACVPTKCEPGCVCAEGLYENAYGQCVPPEECPCEFSGVSYPGGAELHTDCRTCSCSRGRWACQQGTHCPSTCTLYGEGHVITFDGQRFVFDGNCEYILATDVCGVNYSQPTFKILTENVICGNSGVTCSRAIKIFLGGLSVVLADRNYTVTGEEPHVQLGVTPGALSLVVDISIPGRYNLTLIWNRHMTILIRIARASQDPLCGLCGNFNGNMKDDFETRSRYVASSELELVNSWKESPLCGDVSFVTDPCSLNAFRRSWAERKCSVINSQTFATCHSKVYHLPYYEACVRDACGCDSGGDCECLCDAVAAYAQACLDKGVCVDWRTPAFCPIYCGFYNTHTQDGHGEYQYTQEANCTWHYQPCLCPSQPQSVPGSNIEGCYNCSQDEYFDHEEGVCVPCMPPTTPQPPTTPQLPTTGSRPTQVWPMTGTSTTIGLLSSTGPSPSSNHTPASPTQTPLLPATLTSSKPTASSGEPPRPTTAVTPQATSGLPPTATLRSTATKPTVTQATTRATASTASPATTSTAQSTTRTTMTLPTPATSGTSPTLPKSTNQELPGTTATQTTGPRPTPASTTGPTTPQPGQPTRPTATETTQTRTTTEYTTPQTPHTTHSPPTAGSPVPSTGPVTATSFHATTTYPTPSHPETTLPTHVPPFSTSLVTPSTHTVITPTHAQMASSASNHSAPTGTIPPPTTLKATGSTHTAPPITPTTSGTSQAHSSFSTNKTPTSLHSHTSSTHHPEVTPTSTTSITPNPTSTRTRTPMAHTNSATSSRPPTPFTTHSPPTGSSPISSTGPMTAPSFHATTTYPTPSHPQTTLPTHVPSFSTSLVTPSTHIVITPTHAQMATSASIHSMQTGTIPPPTTIKATGSTHTAPPMTPTTSGTSQSLSSFSTAKTSTSLPYHTSSTHHPEVTPTSTTNITPKHTSTGTRTPVAHTTSATSSRLPTPFTTHSPPTGSSPISSTDHHYLSNPITPSDHTSHSRSTFLHLLGDSKYSQGHHPYPCTDGHFCLHPLNANRAPFLPLTTLMNTGSTHTAPLITVTTSRTSQVHSSFSTAKTSTSLLSHASSTHHPEITTNSTTTITPNPTSTGTGTPVAHTTSATSSRLTTTLHHTLPTYREQSLLFHRSYDCNILPDHHYLSNPITPSDHTSHSRSTFLHLFSDSKYSHSHHPYPCTDVHFCLDPLNANSHQPYHQAPWSHLVAYHTVPDQLPHCPWKHPCFCPGIFSRDTYAHLTRNHPGTGSLACIDLHQATTPQLPSWSLTWVAARCCKLRESWFGSLPETGTWVQGVTREVTPRSRGEGAGTSWEGRAAGEGRAYGSTQSPDPPGESPLQRAAGAHGAPATPYVPLWGHWHGVLGPPAGPGSGQPERPMPTGVCSVREQQEEITFKGCMANVTVTRCEGACISAASFNIITQQVDARCSCCRPLHSYEQQLELPCPDPSTPGRRLVLTLQVFSHCVCSSVACGD

SEQ ID NO: 5 - protein, 2264 aa, homo sapiens
MVQRWLLLSCCGALLSAGLANTSYTSPGLQRLKDSPQTAPDKGQCSTWGAGHFSTFDHHVYDFSGTCNYIFAATCKDAFPSFSVQLRRGPDGSISRIIVELGASVVTVSEAIISVKDIGVISLPYTSNGLQITPFGQSVRLVAKQLELELEVVWGPDSHLMVLVERKYMGQMCGLCGNFDGKVTNEFVSEEGKFLEPHKFAALQKLDDPGEICTFQDIPSTHVRQAQHARGCTQLLTLVAPECSVSKEPFVLSCQADVAAAPQPGPQNSSYATLSEYSRQCSMVGQPVALRSPGLCSVGQCPANQVYQECGSACVKTCSNSEHSCSSSCTFGCFCPEGTDLNDLSNNHTCVPVTQCPCVLHGAMYAPGEVTIAACQTCRCTLGRWVCTERPCPGHCSLEGGSFVTTFDARPYRFHGTCTYILLQSPQLPEDGALMAVYDKSGVSHSETSLVAVVYLSRQDKIVISQDEVVTNNGEAKWLPYKTRNITVFRQTSTHLQMATSFGLELVVQLRPIFQAYVTVGPQFRGQTRGLCGNFNGDTTDDFTTSMGIAEGTASLFVDSWRAGNCPDALERETDPCSMSQLNKVCAETHCSMLLRTGTVFERCHATVNPAPIYKRCMYQACNYEETFPHICAALGDYVHACSLRGVLLWGWRSSVDNCTIPCTGNTTFSYNSQACERTCLSLSDRATECHHSAVPVDGCNCPDGTYLNQKGECVRKAQCPCILEGYKFILAEQSTVINGITCHCINGRLSCPQRLQMFLASCQAPKTFKSCSQSSENKFGAACAPTCQMLATGVACVPTKCEPGCVCAEGLYENAYGQCVPPEECPCEFSGVSYPGGAELHTDCRTCSCSRGRWACQQGTHCPSTCTLYGEGHVITFDGQRFVFDGNCEYILATDVCGVNYSQPTFKILTENVICGNSGVTCSRAIKIFLGGLSVVLADRNYTVTGEEPHVQLGVTPGALSLVVDISIPGRYNLTLIWNRHMTILIRIARASQDPLCGLCGNFNGNMKDDFETRSRYVASSELELVNSWKESPLCGDVSFVTDPCSLNAFRRSWAERKCSVINSQTFATCHSKVYHLPYYEACVRDACGCDSGGDCECLCDAVAAYAQACLDKGVCVDWRTPAFCPIYCGFYNTHTQDGHGEYQYTQEANCTWHYQPCLCPSQPQSVPGSNIEGCYNCSQDEYFDHEEGVCVPCMPPTTPQPPTTPQLPTTGSRPTQVWPMTGTSTTIGLLSSTGPSPSSNHTPASPTQTPLLPATLTSSKPTASSGEPPRPTTAVTPQATSGLPPTATLRSTATKPTVTQATTRATASTASPATTSTAQSTTRTTMTLPTPATSGTSPTLPKSTNQELPGTTATQTTGPRPTPASTTGPTTPQPGQPTRPTATETTQTRTTTEYTTPQTPHTTHSPPTAGSPVPSTGPVTATSFHATTTYPTPSHPETTLPTHVPPFSTSLVTPSTHTVITPTHAQMASSASNHSAPTGTIPPPTTLKATGSTHTAPPITPTTSGTSQAHSSFSTNKTPTSLHSHTSSTHHPEVTPTSTTSITPNPTSTRTRTPMAHTNSATSSRPPTPFTTHSPPTGSSPISSTGPMTAPSFHATTTYPTPSHPQTTLPTHVPSFSTSLVTPSTHIVITPTHAQMATSASIHSMQTGTIPPPTTIKATGSTHTAPPMTPTTSGTSQSLSSFSTAKTSTSLPYHTSSTHHPEVTPTSTTNITPKHTSTGTRTPVAHTTSATSSRLPTPFTTHSPPTGSSPISSTDHHYLSNPITPSDHTSHSRSTFLHLLGDSKYSQGHHPYPCTDGHFCLHPLNANRAPFLPLTTLMNTGSTHTAPLITVTTSRTSQVHSSFSTAKTSTSLLSHASSTHHPEITTNSTTTITPNPTSTGTGTPVAHTTSATSSRLTTTLHHTLPTYREQSLLFHRSYDCNILPDHHYLSNPITPSDHTSHSRSTFLHLFSDSKYSHSHHPYPCTDVHFCLDPLNANSHQPYHQAPWSHLVAYHTVPDQLPHCPWKHPCFCPGIFSRDTYAHLTRNHPGTGSLACIDLHQATTPQLPSWSLTWVAARCCKLRESWFGSLPETGTWVQGVTREVTPRSRGEGAGTSWEGRAAGEGRAYGSTQSPDPPGESPLQRAAGAHGAPATPYVPLWGHWHGVLGPPAGPGSGQPERPMPTGVCSVREQQEEITFKGCMANVTVTRCEGACISAASFNIITQQVDARCSCCRPLHSYEQQLELPCPDPSTPGRRLVLTLQVFSHCVCSSVACGDHHHHHH

SEQ ID NO: 6 - DNA, 6684 bp, homo sapiens
  CDS (1)..(6684)
atgagtgttggccggaggaagctggccctgctctgggccctggctctcgctctggcctgcacccggcacacaggccatgcccaggatggctcctccgaatccagctacaagcaccaccctgccctctctcctatcgcccgggggcccagcggggtcccgctccgtggggcgactgtcttcccatctctgaggaccatccctgtggtacgagcctccaacccggcgcacaacgggcgggtgtgcagcacctggggcagcttccactacaagaccttcgacggcgacgtcttccgcttccccggcctctgcaactacgtgttctccgagcactgcggtgccgcctacgaggattttaacatccagctacgccgcagccaggagtcagcggcccccacgctgagcagggtcctcatgaaggtggatggcgtggtcatccagctgaccaagggctccgtcctggtcaacggccacccggtcctgctgcccttcagccagtctggggtcctcattcagcagagcagcagctacaccaaggtggaggccaggctgggccttgtcctcatgtggaaccacgatgacagcctgctgctggagctggacaccaaatacgccaacaagacctgtgggctctgtggggacttcaacgggatgcccgtggtcagcgagctcctctcccacaacaccaagctgacacccatggaattcgggaacctgcagaagatggacgaccccacggagcagtgtcaggaccctgtccctgaacccccgaggaactgctccactggctttggcatctgtgaggagctcctgcacggccagctgttctctggctgcgtggccctggtggacgtcggcagctacctggaggcttgcaggcaagacctctgcttctgtgaagacaccgacctgctcagctgcgtctgccacacccttgccgagtactcccggcagtgcacccatgcaggggggttgccccaggactggcggggccctgacttctgcccccagaagtgccccaacaacatgcagtaccacgagtgccgctccccctgtgcagacacctgctccaaccaggagcactcccgggcctgtgaggaccactgtgtggccggctgcttctgccctgaggggacggtgcttgacgacatcggccagaccggctgtgtccctgtgtcaaagtgtgcctgcgtctacaacggggctgcctatgccccaggggccacctactccacagactgcaccaactgcacctgctccggaggccggtggagctgccaggaggttccatgcccgggtacctgctctgtgcttggaggtgcccacttctcaacgtttgacgggaagcaatacacggtgcacggcgactgcagctatgtgctgaccaagccctgtgacagcagtgccttcactgtactggctgagctgcgcaggtgcgggctgacggacagcgagacctgcctgaagagcgtgacactgagcctggatggggtgcagacggtggtggtgatcaaggccagtggggaagtgttcctgaaccagatctacacccagctgcccatctctgcagccaacgtcaccatcttcagaccctcaaccttcttcatcatcgcccagaccagcctgggcctgcagctgaacctgcagctggtgcccaccatgcagctgttcatgcagctggcgcccaagctccgtgggcagacctgcggtctctgtgggaacttcaacagcatccaggccgatgacttccggaccctcagtggggtggtggaggccaccgctgcggccttcttcaacaccttcaagacccaggccgcctgccccaacatcaggaacagcttcgaggacccctgctctctgagcgtggagaatgtgtgtgctgcgcccatggtgttctttgactgccgaaatgccacgcccggggacacaggggctggctgtcagaagagctgccacacactggacatgacctgttggtgtctgctggccctgcagtacagcccccagtgtgtgcctggctgcgtgtgccccgacgggctggtggcggacggcgagggcggctgcatcactgcggaggactgcccctgcgtgcacaatgaggccagctaccgggccggccagaccatccgggtgggctgcaacacctgcacctgtgacagcaggatgtggcggtgcacagatgacccctgcctggccacctgcgccgtgtacggggacggccactacctcaccttcgacggacagagctacagcttcaacggagactgcgagtacacgctggtgcagaaccactgtggcgggaaagacagcacccaggactcctttcgtgttgtcaccgagaacgtcccctgcggcaccacagggaccacctgctccaaggccatcaagattttcctggggggcttcgagctgaagctaagccatgggaaggtggaggtgatcgggacggacgagagccaggaggtgccatacaccatccggcagatgggcatctacctggtggtggacaccgacattggcctggtgctgctgtgggacaagaagaccagcatcttcatcaacctcagccccgagttcaagggcagggtctgcggcctgtgtgggaacttcgacgacatcgccgttaatgactttgccacgcggagccggtctgtggtgggggacgtgctggagtttgggaacagctggaagctctccccctcctgcccagatgccctggcgcccaaggacccctgcacggccaaccccttccgcaagtcctgggcccagaagcagtgcagcatcctccacggccccaccttcgccgcctgccacgcacacgtggagccggccaggtactacgaggcctgcgtgaacgacgcgtgcgcctgcgactccgggggtgactgcgagtgcttctgcacggctgtggccgcctacgcccaggcctgccatgaagtaggcctgtgtgtgtcctggcggaccccgagcatctgccctctgttctgcgactactacaaccccgaaggccagtgcgagtggcactaccagccctgcggggtgccctgcctgcgcacctgccggaacccccgtggagactgcctgcgggacgtccggggcctggaagccagcacaacctctggtcctggaacttctctcagccctgttcccaccacgagcacaacctctgctcctacaactagcacaacctctggtcctggaactactcccagccctgttcccaccaccagcacaacctctgctcctacaaccagcacgacctctggtcctggaactactcccagccccgttcccaccaccagcacaacccctgtttcaaagaccagcacaagccatctttctgtatccaagacaacccactcccaaccagtcaccagtgactgtcatcctctgtgcgcctggacaaagtggttcgacgtggacttcccatcccctggaccccacggcggggacaaggaaacctacaacaacatcatcaggagtggggaaaaaatctgccgccgacctgaggagatcaccaggctccagtgccgagccgagagccacccggaggtgaacattgaacacctgggtcaggtggtgcagtgcagccgtgaagagggcctggtgtgccggaaccaggaccagcagggacccttcaagatgtgcctcaactacgaggtgcgcgtgctctgctgcgagacccccagaggctgcccggtgacctctgtgaccccatatgggacttctcctaccaatgctctgtatccttccctgtctacttccatggtatccgcctccgtggcatccacctctgtggcatccagctctgtggcatccagctctgtggcttactccacccaaacctgcttctgcaacgtggctgaccggctctaccctgcaggatccaccatataccgccacagagacctcgctggccattgctattatgccctgtgtagccaggactgccaagtggtcagaggggttgacagtgactgtccgtccaccacgctgcctcctgccccagccacgtccccttcaatatccacctccgagcccgtcactgagctgggatgcccaaatgcggttccccccagaaagaaaggtgagacctgggccacacccaactgctccgaggccacctgtgagggcaacaacgtcatctccctgcgcccgcgcacgtgcccgagggtggagaagcccacttgtgccaacggctacccggctgtgaaggtggctgaccaagatggctgctgccatcactaccagtgccagtgtgtgtgcagcggctggggtgacccccactacatcaccttcgacggcacctactacaccttcctggacaactgcacgtacgtgctggtgcagcagattgtgcccgtgtatggccacttccgcgtgctcgtcgacaactacttctgcggtgcggaggacgggctctcctgcccgaggtccatcatcctggagtaccaccaggaccgcgtggtgctgacccgcaagccagtccacggggtgatgacgaacgaggtgggggcgcgcccgatcatcttcaacaacaaggtggtcagccccggcttccggaaaaacggcatcgtggtctcgcgcatcggcgtcaagatgtacgcgaccatcccggagctgggagtccaggtcatgttctccggcctcatcttctccgtggaggtgcccttcagcaagtttgccaacaacaccgagggccagtgcggcacttgcaccaacgacaggaaggatgagtgccgcacgcctagggggacggtggtcgcttcctgctccgagatgtccggcctctggaacgtgagcatccctgaccagccagcctgccaccggcctcacccgacgcccaccacggtcgggcccaccacagttgggtctaccacggtcgggcccaccacagttgggtctaccaccgtcgggcccaccacaccgcctgctccgtgcctgccatcacccatctgccagctgattctgagcaaggtctttgagccgtgccacactgtgatccccccactgctgttctatgagggctgcgtctttgaccggtgccacatgacggacctggatgtggtgtgctccagcctggagctgtacgcggcactctgtgcgtcccacgacatctgcatcgattggagaggccggaccggccacatgtgcccattcacctgcccagccgacaaggtgtaccagccctgcggcccgagcaacccctcctactgctacgggaatgacagcgccagcctcggggctctgccggaggccggccccatcaccgaaggctgcttctgtccggagggcatgaccctcttcagcaccagtgcccaagtctgcgtgcccacgggctgccccaggtgtctggggccccacggagagccggtgaaggtgggccacaccgtcggcatggactgccaggagtgcacgtgtgaggcggccacgtggacgctgacctgccgacccaagctctgcccgctgccccctgcctgccccctgcccggcttcgtgcctgtgcctgcagccccacaggccggccagtgctgcccccagtacagctgcgcctgcaacaccagccgctgccccgcgcccgtgggctgtcctgagggcgcccgcgcgatcccgacctaccaggagggggcctgctgcccagtccaaaactgcagctggacagtgtgcagcatcaacgggaccctgtaccagcccggcgccgtggtctcctcgagcctgtgcgaaacctgcaggtgtgagctgccgggtggccccccatcggacgcgtttgtggtcagctgtgagacccagatctgcaacacacactgccctgtgggcttcgagtaccaggagcagagcgggcagtgctgtggcacctgtgtgcaggtcgcctgtgtcaccaacaccagcaagagccccgcccacctcttctaccctggcgagacctggtcagacgcagggaaccactgtgtgacccaccagtgtgagaagcaccaggatgggctcgtggtggtcaccacgaagaaggcgtgccccccgctcagctgttctctggtgaggtccaggatccccgctccagccaaggggggcttcacccctagatgggtttggggggctgtgatcatccctgcagcgccagcagacaccccctcctgcttggggctgtccactcctgagcctggccccatgtccccatccctcacttctgtgggggccgccgagcgcctcggcactgagggcgcccctctgtcggcacaggacgaggcccgcatgagcaaggacggctgctgccgcttctgcccgccgcccccgcccccgtaccagaaccagtcgacctgtgctgtgtaccataggagcctgatcatccagcagcagggctgcagctcctcggagcccgtgcgcctggcttactgccgggggaactgtggggacagctcttccatgtactcgctcgagggcaacacggtggagcacaggtgccagtgctgccaggagctgcggacctcgctgaggaatgtgaccctgcactgcaccgacggctccagccgggccttcagctacaccgaggtggaagagtgcggctgcatgggccggcggtgccctgcgccgggcgacacccagcactcggaggaggcggaacccgagcccagccaggaggcagagagtgggagctgggagagaggcgtcccagtgtcccccatgcactga

SEQ ID NO: 7 - protein, 2227 aa, homo sapiens
MSVGRRKLALLWALALALACTRHTGHAQDGSSESSYKHHPALSPIARGPSGVPLRGATVFPSLRTIPVVRASNPAHNGRVCSTWGSFHYKTFDGDVFRFPGLCNYVFSEHCGAAYEDFNIQLRRSQESAAPTLSRVLMKVDGVVIQLTKGSVLVNGHPVLLPFSQSGVLIQQSSSYTKVEARLGLVLMWNHDDSLLLELDTKYANKTCGLCGDFNGMPVVSELLSHNTKLTPMEFGNLQKMDDPTEQCQDPVPEPPRNCSTGFGICEELLHGQLFSGCVALVDVGSYLEACRQDLCFCEDTDLLSCVCHTLAEYSRQCTHAGGLPQDWRGPDFCPQKCPNNMQYHECRSPCADTCSNQEHSRACEDHCVAGCFCPEGTVLDDIGQTGCVPVSKCACVYNGAAYAPGATYSTDCTNCTCSGGRWSCQEVPCPGTCSVLGGAHFSTFDGKQYTVHGDCSYVLTKPCDSSAFTVLAELRRCGLTDSETCLKSVTLSLDGVQTVVVIKASGEVFLNQIYTQLPISAANVTIFRPSTFFIIAQTSLGLQLNLQLVPTMQLFMQLAPKLRGQTCGLCGNFNSIQADDFRTLSGVVEATAAAFFNTFKTQAACPNIRNSFEDPCSLSVENVCAAPMVFFDCRNATPGDTGAGCQKSCHTLDMTCWCLLALQYSPQCVPGCVCPDGLVADGEGGCITAEDCPCVHNEASYRAGQTIRVGCNTCTCDSRMWRCTDDPCLATCAVYGDGHYLTFDGQSYSFNGDCEYTLVQNHCGGKDSTQDSFRVVTENVPCGTTGTTCSKAIKIFLGGFELKLSHGKVEVIGTDESQEVPYTIRQMGIYLVVDTDIGLVLLWDKKTSIFINLSPEFKGRVCGLCGNFDDIAVNDFATRSRSVVGDVLEFGNSWKLSPSCPDALAPKDPCTANPFRKSWAQKQCSILHGPTFAACHAHVEPARYYEACVNDACACDSGGDCECFCTAVAAYAQACHEVGLCVSWRTPSICPLFCDYYNPEGQCEWHYQPCGVPCLRTCRNPRGDCLRDVRGLEASTTSGPGTSLSPVPTTSTTSAPTTSTTSGPGTTPSPVPTTSTTSAPTTSTTSGPGTTPSPVPTTSTTPVSKTSTSHLSVSKTTHSQPVTSDCHPLCAWTKWFDVDFPSPGPHGGDKETYNNIIRSGEKICRRPEEITRLQCRAESHPEVNIEHLGQVVQCSREEGLVCRNQDQQGPFKMCLNYEVRVLCCETPRGCPVTSVTPYGTSPTNALYPSLSTSMVSASVASTSVASSSVASSSVAYSTQTCFCNVADRLYPAGSTIYRHRDLAGHCYYALCSQDCQVVRGVDSDCPSTTLPPAPATSPSISTSEPVTELGCPNAVPPRKKGETWATPNCSEATCEGNNVISLRPRTCPRVEKPTCANGYPAVKVADQDGCCHHYQCQCVCSGWGDPHYITFDGTYYTFLDNCTYVLVQQIVPVYGHFRVLVDNYFCGAEDGLSCPRSIILEYHQDRVVLTRKPVHGVMTNEVGARPIIFNNKVVSPGFRKNGIVVSRIGVKMYATIPELGVQVMFSGLIFSVEVPFSKFANNTEGQCGTCTNDRKDECRTPRGTVVASCSEMSGLWNVSIPDQPACHRPHPTPTTVGPTTVGSTTVGPTTVGSTTVGPTTPPAPCLPSPICQLILSKVFEPCHTVIPPLLFYEGCVFDRCHMTDLDVVCSSLELYAALCASHDICIDWRGRTGHMCPFTCPADKVYQPCGPSNPSYCYGNDSASLGALPEAGPITEGCFCPEGMTLFSTSAQVCVPTGCPRCLGPHGEPVKVGHTVGMDCQECTCEAATWTLTCRPKLCPLPPACPLPGFVPVPAAPQAGQCCPQYSCACNTSRCPAPVGCPEGARAIPTYQEGACCPVQNCSWTVCSINGTLYQPGAVVSSSLCETCRCELPGGPPSDAFVVSCETQICNTHCPVGFEYQEQSGQCCGTCVQVACVTNTSKSPAHLFYPGETWSDAGNHCVTHQCEKHQDGLVVVTTKKACPPLSCSLVRSRIPAPAKGGFTPRWVWGAVIIPAAPADTPSCLGLSTPEPGPMSPSLTSVGAAERLGTEGAPLSAQDEARMSKDGCCRFCPPPPPPYQNQSTCAVYHRSLIIQQQGCSSSEPVRLAYCRGNCGDSSSMYSLEGNTVEHRCQCCQELRTSLRNVTLHCTDGSSRAFSYTEVEECGCMGRRCPAPGDTQHSEEAEPEPSQEAESGSWERGVPVSPMH

SEQ ID NO: 8 - protein, 2202 aa, homo sapiens
HAQDGSSESSYKHHPALSPIARGPSGVPLRGATVFPSLRTIPVVRASNPAHNGRVCSTWGSFHYKTFDGDVFRFPGLCNYVFSEHCGAAYEDFNIQLRRSQESAAPTLSRVLMKVDGVVIQLTKGSVLVNGHPVLLPFSQSGVLIQQSSSYTKVEARLGLVLMWNHDDSLLLELDTKYANKTCGLCGDFNGMPVVSELLSHNTKLTPMEFGNLQKMDDPTEQCQDPVPEPPRNCSTGFGICEELLHGQLFSGCVALVDVGSYLEACRQDLCFCEDTDLLSCVCHTLAEYSRQCTHAGGLPQDWRGPDFCPQKCPNNMQYHECRSPCADTCSNQEHSRACEDHCVAGCFCPEGTVLDDIGQTGCVPVSKCACVYNGAAYAPGATYSTDCTNCTCSGGRWSCQEVPCPGTCSVLGGAHFSTFDGKQYTVHGDCSYVLTKPCDSSAFTVLAELRRCGLTDSETCLKSVTLSLDGVQTVVVIKASGEVFLNQIYTQLPISAANVTIFRPSTFFIIAQTSLGLQLNLQLVPTMQLFMQLAPKLRGQTCGLCGNFNSIQADDFRTLSGVVEATAAAFFNTFKTQAACPNIRNSFEDPCSLSVENVCAAPMVFFDCRNATPGDTGAGCQKSCHTLDMTCWCLLALQYSPQCVPGCVCPDGLVADGEGGCITAEDCPCVHNEASYRAGQTIRVGCNTCTCDSRMWRCTDDPCLATCAVYGDGHYLTFDGQSYSFNGDCEYTLVQNHCGGKDSTQDSFRVVTENVPCGTTGTTCSKAIKIFLGGFELKLSHGKVEVIGTDESQEVPYTIRQMGIYLVVDTDIGLVLLWDKKTSIFINLSPEFKGRVCGLCGNFDDIAVNDFATRSRSVVGDVLEFGNSWKLSPSCPDALAPKDPCTANPFRKSWAQKQCSILHGPTFAACHAHVEPARYYEACVNDACACDSGGDCECFCTAVAAYAQACHEVGLCVSWRTPSICPLFCDYYNPEGQCEWHYQPCGVPCLRTCRNPRGDCLRDVRGLEASTTSGPGTSLSPVPTTSTTSAPTTSTTSGPGTTPSPVPTTSTTSAPTTSTTSGPGTTPSPVPTTSTTPVSKTSTSHLSVSKTTHSQPVTSDCHPLCAWTKWFDVDFPSPGPHGGDKETYNNIIRSGEKICRRPEEITRLQCRAESHPEVNIEHLGQVVQCSREEGLVCRNQDQQGPFKMCLNYEVRVLCCETPRGCPVTSVTPYGTSPTNALYPSLSTSMVSASVASTSVASSSVASSSVAYSTQTCFCNVADRLYPAGSTIYRHRDLAGHCYYALCSQDCQVVRGVDSDCPSTTLPPAPATSPSISTSEPVTELGCPNAVPPRKKGETWATPNCSEATCEGNNVISLRPRTCPRVEKPTCANGYPAVKVADQDGCCHHYQCQCVCSGWGDPHYITFDGTYYTFLDNCTYVLVQQIVPVYGHFRVLVDNYFCGAEDGLSCPRSIILEYHQDRVVLTRKPVHGVMTNEVGARPIIFNNKVVSPGFRKNGIVVSRIGVKMYATIPELGVQVMFSGLIFSVEVPFSKFANNTEGQCGTCTNDRKDECRTPRGTVVASCSEMSGLWNVSIPDQPACHRPHPTPTTVGPTTVGSTTVGPTTVGSTTVGPTTPPAPCLPSPICQLILSKVFEPCHTVIPPLLFYEGCVFDRCHMTDLDVVCSSLELYAALCASHDICIDWRGRTGHMCPFTCPADKVYQPCGPSNPSYCYGNDSASLGALPEAGPITEGCFCPEGMTLFSTSAQVCVPTGCPRCLGPHGEPVKVGHTVGMDCQECTCEAATWTLTCRPKLCPLPPACPLPGFVPVPAAPQAGQCCPQYSCACNTSRCPAPVGCPEGARAIPTYQEGACCPVQNCSWTVCSINGTLYQPGAVVSSSLCETCRCELPGGPPSDAFVVSCETQICNTHCPVGFEYQEQSGQCCGTCVQVACVTNTSKSPAHLFYPGETWSDAGNHCVTHQCEKHQDGLVVVTTKKACPPLSCSLVRSRIPAPAKGGFTPRWVWGAVIIPAAPADTPSCLGLSTPEPGPMSPSLTSVGAAERLGTEGAPLSAQDEARMSKDGCCRFCPPPPPPYQNQSTCAVYHRSLIIQQQGCSSSEPVRLAYCRGNCGDSSSMYSLEGNTVEHRCQCCQELRTSLRNVTLHCTDGSSRAFSYTEVEECGCMGRRCPAPGDTQHSEEAEPEPSQEAESGSWERGVPVSPMH

SEQ ID NO: 9 - protein, 2233 aa, homo sapiens
MSVGRRKLALLWALALALACTRHTGHAQDGSSESSYKHHPALSPIARGPSGVPLRGATVFPSLRTIPVVRASNPAHNGRVCSTWGSFHYKTFDGDVFRFPGLCNYVFSEHCGAAYEDFNIQLRRSQESAAPTLSRVLMKVDGVVIQLTKGSVLVNGHPVLLPFSQSGVLIQQSSSYTKVEARLGLVLMWNHDDSLLLELDTKYANKTCGLCGDFNGMPVVSELLSHNTKLTPMEFGNLQKMDDPTEQCQDPVPEPPRNCSTGFGICEELLHGQLFSGCVALVDVGSYLEACRQDLCFCEDTDLLSCVCHTLAEYSRQCTHAGGLPQDWRGPDFCPQKCPNNMQYHECRSPCADTCSNQEHSRACEDHCVAGCFCPEGTVLDDIGQTGCVPVSKCACVYNGAAYAPGATYSTDCTNCTCSGGRWSCQEVPCPGTCSVLGGAHFSTFDGKQYTVHGDCSYVLTKPCDSSAFTVLAELRRCGLTDSETCLKSVTLSLDGVQTVVVIKASGEVFLNQIYTQLPISAANVTIFRPSTFFIIAQTSLGLQLNLQLVPTMQLFMQLAPKLRGQTCGLCGNFNSIQADDFRTLSGVVEATAAAFFNTFKTQAACPNIRNSFEDPCSLSVENVCAAPMVFFDCRNATPGDTGAGCQKSCHTLDMTCWCLLALQYSPQCVPGCVCPDGLVADGEGGCITAEDCPCVHNEASYRAGQTIRVGCNTCTCDSRMWRCTDDPCLATCAVYGDGHYLTFDGQSYSFNGDCEYTLVQNHCGGKDSTQDSFRVVTENVPCGTTGTTCSKAIKIFLGGFELKLSHGKVEVIGTDESQEVPYTIRQMGIYLVVDTDIGLVLLWDKKTSIFINLSPEFKGRVCGLCGNFDDIAVNDFATRSRSVVGDVLEFGNSWKLSPSCPDALAPKDPCTANPFRKSWAQKQCSILHGPTFAACHAHVEPARYYEACVNDACACDSGGDCECFCTAVAAYAQACHEVGLCVSWRTPSICPLFCDYYNPEGQCEWHYQPCGVPCLRTCRNPRGDCLRDVRGLEASTTSGPGTSLSPVPTTSTTSAPTTSTTSGPGTTPSPVPTTSTTSAPTTSTTSGPGTTPSPVPTTSTTPVSKTSTSHLSVSKTTHSQPVTSDCHPLCAWTKWFDVDFPSPGPHGGDKETYNNIIRSGEKICRRPEEITRLQCRAESHPEVNIEHLGQVVQCSREEGLVCRNQDQQGPFKMCLNYEVRVLCCETPRGCPVTSVTPYGTSPTNALYPSLSTSMVSASVASTSVASSSVASSSVAYSTQTCFCNVADRLYPAGSTIYRHRDLAGHCYYALCSQDCQVVRGVDSDCPSTTLPPAPATSPSISTSEPVTELGCPNAVPPRKKGETWATPNCSEATCEGNNVISLRPRTCPRVEKPTCANGYPAVKVADQDGCCHHYQCQCVCSGWGDPHYITFDGTYYTFLDNCTYVLVQQIVPVYGHFRVLVDNYFCGAEDGLSCPRSIILEYHQDRVVLTRKPVHGVMTNEVGARPIIFNNKVVSPGFRKNGIVVSRIGVKMYATIPELGVQVMFSGLIFSVEVPFSKFANNTEGQCGTCTNDRKDECRTPRGTVVASCSEMSGLWNVSIPDQPACHRPHPTPTTVGPTTVGSTTVGPTTVGSTTVGPTTPPAPCLPSPICQLILSKVFEPCHTVIPPLLFYEGCVFDRCHMTDLDVVCSSLELYAALCASHDICIDWRGRTGHMCPFTCPADKVYQPCGPSNPSYCYGNDSASLGALPEAGPITEGCFCPEGMTLFSTSAQVCVPTGCPRCLGPHGEPVKVGHTVGMDCQECTCEAATWTLTCRPKLCPLPPACPLPGFVPVPAAPQAGQCCPQYSCACNTSRCPAPVGCPEGARAIPTYQEGACCPVQNCSWTVCSINGTLYQPGAVVSSSLCETCRCELPGGPPSDAFVVSCETQICNTHCPVGFEYQEQSGQCCGTCVQVACVTNTSKSPAHLFYPGETWSDAGNHCVTHQCEKHQDGLVVVTTKKACPPLSCSLVRSRIPAPAKGGFTPRWVWGAVIIPAAPADTPSCLGLSTPEPGPMSPSLTSVGAAERLGTEGAPLSAQDEARMSKDGCCRFCPPPPPPYQNQSTCAVYHRSLIIQQQGCSSSEPVRLAYCRGNCGDSSSMYSLEGNTVEHRCQCCQELRTSLRNVTLHCTDGSSRAFSYTEVEECGCMGRRCPAPGDTQHSEEAEPEPSQEAESGSWERGVPVSPMHHHHHHH

(19) United States
(12) Patent Application Publication
(10) Pub. No.: US 2006/0150262 A1
(43) Pub. Date: Jul. 6, 2006

(54) Novel mucin-like polypeptides

(76) Inventors: Jadwiga Bienkowska, Cambridge, MA (US); Gregg McAllister, Charlestown, MA (US)

Correspondence Address: HOWREY LLP, C/O IP DOCKETING DEPARTMENT, 2941 FAIRVIEW PARK DR, SUITE 200, FALLS CHURCH, VA, 22042-2924, US

(21) Appl. No.: 10/544,731

(22) PCT Filed: Feb. 4, 2004
(86) PCT No.: PCT/EP04/50082

§ 371 Date: Aug. 5, 2005

Related U.S. Application Data
(60) Provisional application No. 60/445,217, filed on Feb. 5, 2003.

(51) Int. Cl. A01K 67/027; C07H 21/04; C12P 21/06; A61K 38/17; C07K 14/705

(52) U.S. Cl. 800/8; 514/2; 435/69.1; 435/320.1; 435/325; 530/395; 536/23.5

(57) ABSTRACT

The present invention discloses open reading frame (ORFs) in human genome encoding for novel mucin-like polypeptides, and reagents related thereto including variants, mutants and fragments of said polypeptides, as well as ligands and antagonists directed against them. The invention provides methods for identifying and making these molecules, for preparing pharmaceutical compositions containing them, and for using them in the diagnosis, prevention and treatment of disease.

### FIELD OF THE INVENTION

The present invention relates to nucleic acid sequences identified in human genome as encoding for novel polypeptides, more specifically for mucin-like polypeptides. All publications, patents and patent applications cited herein are incorporated in full by reference.

### BACKGROUND OF THE INVENTION

Many novel polypeptides have been already identified by applying strict homology criteria to known polypeptides of the same family. However, since the actual content in polypeptide-encoding sequences in the human genome for mucin-like polypeptides (and for any other protein family) is still unknown, the possibility still exists to identify DNA sequence encoding polypeptide having mucin-like polypeptide activities by applying alternative and less strict homology/structural criteria to the totality of Open Reading Frames (ORFs, that is, genomic sequences containing consecutive triplets of nucleotides coding for amino acids, not interrupted by a termination codon and potentially translatable in a polypeptide) present in the human genome.

The epithelial surface of the respiratory, gastrointestinal and reproductive tracts is coated with mucus, which is secreted by specialized epithelial cells, e.g. goblet cells and submucosal gland cells. Mucus secretions provide important protective and lubricative functions varying among the tissues. Most of the properties of mucus have been attributed to mucins. To date, several human mucin genes (MUC1, MUC2, MUC3, MUC4, MUC5AC, MUC5B, MUC6, MUC7, MUC8, MUC9, MUC10, MUC11, MUC12, MUC13, MUC15, MUC16, MUC17, MUC18, MUC19, MUC20) have been identified (for reviews, see Gendler, S. J. and Spicer, A. P. (1995) Annu. Rev. Physiol. 57, 607-634 and Shankar, V. et al., (1997) Am. J. Respir. Cell Mol. Biol. 16, 232-241). Four of the mucin genes, MUC2, MUC5AC, MUC5B, and MUC6, have been mapped to chromosome 11p15.5.

All mucin genes share common features, including tandemly repeated sequences flanked by non-repeat regions. They encode peptides rich in threonine and serine which support the numerous O-glycan chains. Cysteine-rich domains have been reported in the N- and C-terminal regions of MUC2, the C-terminal region of MUC5B, the C-terminal region of MUC6, in NP3a, L31 and HGM-1. The C-terminal regions of MUC2 and MUC5B, NP3a and L31 exhibit striking sequence similarities with the D4, B. C and CK domains of the human von Willebrand factor (vWF). Other cysteine-rich domains, designated cysteine-rich subdomains, have been reported in the central repetitive domains of MUC5AC and MUC5B.

Qualitative and quantitative alterations in the expression of the MUC5AC gene have been reported in both preneoplastic and rectosigmoid villous adenomas, but the gene is absent from normal intestine and colon cancers. The expression level of MUC5AC in rectosigmoid villous adenomas is correlated to the degree of dysplasia. Moreover, MUC5AC is expressed in embryonic and foetal intestine. Likewise, MUC5AC mRNAs are detectable in pancreatic cancers but not in normal pancreas.

Although MUC5AC and MUC5B have been shown by physical mapping and expression pattern to be distinct mucin genes, confusion has been introduced in the nomenclature with the cloning of a new cDNA NP3a that has been designated as MUC5.

It is clear that the identification of novel mucin-like proteins is of significant importance in increasing understanding of the underlying pathways that lead to certain disease states in which these proteins are implicated, and in developing more effective gene or drug therapies to treat these disorders.

### SUMMARY OF THE INVENTION

The invention is based upon the identification of Open Reading Frames (ORFs) in the human genome encoding novel mucin-like polypeptides. The polypeptides will be referred to herein as the SCS0004 polypeptides and the SCS0005 polypeptide.

Accordingly, the invention provides isolated SCS0004, SCS0004 variant and SCS0005 polypeptides having the amino acid sequence given by SEQ ID NO: 2, SEQ ID NO: 3 and SEQ ID NO: 7 respectively, and their mature forms, histidine tagged forms, variants, and fragments, as polypeptides having the activity of mucin-like polypeptides. The invention includes also the nucleic acids encoding them, vectors containing such nucleic acids, and cell containing these vectors or nucleic acids, as well as other related reagents such as fusion proteins, ligands, and antagonists.

The invention provides methods for identifying and making these molecules, for preparing pharmaceutical compositions containing them, and for using them in the diagnosis, prevention and treatment of diseases.

### BRIEF DESCRIPTION OF THE FIGURES

FIG. 1: Alignment of SCS0004 with AAQ82434 (MUC6)

FIG. 2: Alignment of SCS0004 variant with AAQ82434

FIG. 3: Alignment of SCS0005 with MU5A_HUMAN (MUC5AC)

FIG. 4: SMART Domains alignment of SCS0004, SCS0004 variant, AAQ82434 and MU5A_HUMAN polypeptides. Transmembrane segments as predicted by the TMHMM2 program [symbol] coiled coil regions determined by the Coils2 program ( [symbol] ) and Segments of low compositional complexity, determined by the SEG program ( [symbol] ) signal peptides determined by the Sigcleave program ( [symbol] ), GPI anchors are indicated by (|). Hits only found by BLAST are indicated by [symbol] for hits in the schnipsel database and [symbol] for hits against PDB. Regions containing repeats detected by Prospero, but not covered by domains are indicated by

[symbol]

### DETAILED DESCRIPTION OF THE INVENTION

In one embodiment, according to a first aspect of the present invention, there is provided an isolated polypeptide having mucin-like activity selected from the group consisting of
a) the amino acid sequence as recited in SEQ ID NO: 2;
b) the mature form of the polypeptide whose sequence is recited in SEQ ID NO: 2;
c) a variant of the amino acid sequence recited in SEQ ID NO: 2, wherein any amino acid specified in the chosen sequence is non-conservatively substituted, provided that no more than 15% of the amino acid residues in the sequence are so changed;
d) an active fragment, precursor, salt, or derivative of the amino acid sequences given in a) to c).

In a second embodiment according to a first aspect of the present invention, there is provided an isolated polypeptide having mucin-like activity selected from the group consisting of
a) the amino acid sequences as recited in SEQ ID NO: 3 or SEQ ID NO: 7;
b) the mature form of the polypeptides whose sequence are recited in SEQ ID NO: 3 (SEQ ID NO:4) or SEQ ID NO: 7 (SEQ ID NO:8);
c) the histidine tagged form of the polypeptides whose sequence are recited in SEQ ID NO: 3 (SEQ ID NO:5) or SEQ ID NO: 7 (SEQ ID NO:9);
d) a variant of the amino acid sequences recited in SEQ ID NO: 3, SEQ ID NO: 4, SEQ ID NO: 5, SEQ ID NO: 7, SEQ ID NO: 8 or SEQ ID NO: 9, wherein any amino acid specified in the chosen sequence is non-conservatively substituted, provided that no more than 15% of the amino acid residues in the sequence are so changed;
e) an active fragment, precursor, salt, or derivative of the amino acid sequences given in a) to d).

The novel polypeptide described herein was identified using cysteine knot domains as query sequences and the final annotation was attributed on the basis of amino acid sequence homology

The totality of amino acid sequences obtained by translating the known ORFs in the human genome were challenged using this consensus sequence, and the positive hits were further screened for the presence of predicted specific structural and functional “signatures” that are distinctive of a polypeptide of this nature, and finally selected by comparing sequence features with known mucin-like polypeptides. Therefore, the novel polypeptides of the invention can be predicted to have mucin-like activities.

The terms “active” and “activity” refer to the mucin-like properties predicted for the mucin-like polypeptide whose amino acid sequence is presented in SEQ ID NO: 2, SEQ ID NO: 3, SEQ ID NO: 4, SEQ ID NO: 5, SEQ ID NO: 7, SEQ ID NO: 8 or SEQ ID NO: 9 in the present application. Mucins can be used for their property of acting as a substrate for mucinase activity.

In a second aspect, the invention provides a purified nucleic acid molecule which encodes a polypeptide of the first aspect of the invention. Preferably, the purified nucleic acid molecule has the nucleic acid sequence as recited in SEQ ID NO: 1 (encoding the mucin-like polypeptide whose amino acid sequence is recited in SEQ ID NO: 2) or SEQ ID NO: 6 (encoding the mucin-like polypeptide whose amino acid sequence is recited in SEQ ID NO:7).

In a third aspect, the invention provides a purified nucleic acid molecule which hydridizes under high stringency conditions with a nucleic acid molecule of the second aspect of the invention.

In a fourth aspect, the invention provides a vector, such as an expression vector, that contains a nucleic acid molecule of the second or third aspect of the invention.

In a fifth aspect, the invention provides a host cell transformed with a vector of the fourth aspect of the invention.

In a sixth aspect, the invention provides a ligand which binds specifically to, and which preferably inhibits the mucin-like activity of a polypeptide of the first aspect of the invention. Ligands to a polypeptide according to the invention may come in various forms, including natural or modified substrates, enzymes, receptors, small organic molecules such as small natural or synthetic organic molecules of up to 2000 Da, preferably 800 Da or less, peptidomimetics, inorganic molecules, peptides, polypeptides, antibodies, structural or functional mimetics of the aforementioned.

In a seventh aspect, the invention provides a compound that is effective to alter the expression of a natural gene which encodes a polypeptide of the first aspect of the invention or to regulate the activity of a polypeptide of the first aspect of the invention.

A compound of the seventh aspect of the invention may either increase (agonise) or decrease (antagonism) the level of expression of the gene or the activity of the polypeptide. Importantly, the identification of the function of the mucin-like polypeptide of the invention allows for the design of screening methods capable of identifying compounds that are effective in the treatment and/or diagnosis of disease.

In an eighth aspect, the invention provides a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, for use in therapy or diagnosis. These molecules may also be used in the manufacture of a medicament for the prevention and treatment of diseases and conditions in which mucin-like polypeptides are implicated such as cell proliferative disorders, autoimmune/inflammatory disorders, cardiovascular disorders, neurological disorders, developmental disorders, metabolic disorders, infections and other pathological conditions.

In a ninth aspect, the invention provides a method of diagnosing a disease in a patient, comprising assessing the level of expression of a natural gene encoding a polypeptide of the first aspect of the invention or the activity of a polypeptide of the first aspect of the invention in tissue from said patient and comparing said level of expression or activity to a control level, wherein a level that is different to said control level is indicative of disease. Such a method will preferably be carried out in vitro. Similar methods may be used for monitoring the therapeutic treatment of disease in a patient, wherein altering the level of expression or activity of a polypeptide or nucleic acid is molecule over the period of time towards a control level is indicative of regression of disease.

A preferred method for detecting polypeptides of the first aspect of the invention comprises the steps of: (a) contacting a ligand, such as an antibody, of the sixth aspect of the invention with a biological sample under conditions suitable for the formation of a ligand-polypeptide complex; and (b) detecting said complex.

A number of different such methods according to the ninth aspect of the invention exist, as the skilled reader will be aware, such as methods of nucleic acid hybridization with short probes, point mutation analysis, polymerase chain reaction (PCR) amplification and methods using antibodies to detect aberrant protein levels. Similar methods may be used on a short or long term basis to allow therapeutic treatment of a disease to be monitored in a patient. The invention also provides kits that are useful in these methods for diagnosing disease.

In a tenth aspect the invention provides for the use of a polypeptide of the first aspect of the invention as a mucin-like protein. Suitable uses include use as a substrate for detecting mucinase activity.

In an eleventh aspect the invention provides a pharmaceutical composition comprising a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, in conjunction with a pharmaceutically-acceptable carrier.

In a twelfth aspect, the present invention provides a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention, for use in the manufacture of a medicament for the diagnosis or treatment of a disease or condition in which mucin-like polypeptides are implicated such as cell proliferative disorders, autoimmune/inflammatory disorders, cardiovascular disorders, neurological disorders, developmental disorders, metabolic disorders, infections and other pathological conditions.

In a thirteenth aspect the invention provides a method of treating a disease in a patient comprising administering to the patient a polypeptide of the first aspect of the invention, or a nucleic acid molecule of the second or third aspect of the invention, or a vector of the fourth aspect of the invention, or a host cell of the fifth aspect of the invention, or a ligand of the sixth aspect of the invention, or a compound of the seventh aspect of the invention.

For diseases in which the expression of a natural gene encoding a polypeptide of the first aspect of the invention, or in which the activity of a polypeptide of the first aspect of the invention, is lower in a diseased patient when compared to the level of expression or activity in a healthy patient the polypeptide, nucleic acid molecule, ligand or compound administered to the patient should be an agonist. Conversely, for diseases in which the expression of the natural gene or activity of the polypeptide is higher in a diseased patient when compared to the level of expression or activity in a healthy patient, the polypeptide, nucleic acid molecule, ligand or compound administered to the patient should be an antagonist Examples of such antagonists include antisense nucleic acid molecules, ribozymes and ligands, such as antibodies.

In a fourteenth aspect, the invention provides transgenic or knockout non-human animals that have been transformed to express higher, lower or absent levels of a polypeptide of the first aspect of the invention. Such transgenic animals are very useful models for the study of disease and may also be used in screening regimes for the identification of compounds that are effective in the treatment or diagnosis of such a disease.

A summary of standard techniques and procedures which may be employed in order to utilise the invention is given below. It will be understood that this invention is not limited to the particular methodology, protocols, cell lines, vectors and reagents described. It is also to be understood that the terminology used herein is for the purpose of describing particular embodiments only and it is not intended that this terminology should limit the scope of the present invention. The extent of the invention is limited only by the terms of the appended claims.

Standard abbreviations for nucleotides and amino acids are used in this specification.

The practice of the present invention will employ, unless otherwise indicated, conventional techniques of molecular biology, microbiology, recombinant DNA technology and immunology, which are within the skill of those working in the art.

Such techniques are explained fully in the literature. Examples of particularly suitable texts for consultation include the following: Sambrook Molecular Cloning; A Laboratory Manual, Second Edition (1989); DNA Cloning, Volumes I and II (D. N Glover ed. 1985); Oligonucleotide Synthesis (M. J. Gait ed. 1984): Nucleic Acid Hybridization (B. D. Hames & S. J. Higgins eds. 1984); Transcription and Translation (B. D. Hames & S. J. Higgins eds. 1984); Animal Cell Culture (R. I. Freshney ed. 1986); Immobilized Cells and Enzymes (IRL Press, 1986); B. Perbal, A Practical Guide to Molecular Cloning (1984); the Methods in Enzymology series (Academic Press, Inc.), especially volumes 154 & 155; Gene Transfer Vectors for Mammalian Cells (J. H. Miller and M. P. Calos eds. 1987, Cold Spring Harbor Laboratory); Immunochemical Methods in Cell and Molecular Biology (Mayer and Walker, eds. 1987, Academic Press, London); Scopes, (1987) Protein Purification: Principles and Practice, Second Edition (Springer Verlag, N.Y.); and Handbook of Experimental Immunology, Volumes I-IV (D. M. Weir and C. C. Blackwell eds. 1986).

The first aspect of the invention includes variants of the amino acid sequence recited in SEQ ID NO: 2, SEQ ID NO: 3, SEQ ID NO: 4, SEQ ID NO: 5, SEQ ID NO: 7, SEQ ID NO: 8 or SEQ ID NO: 9, wherein any amino acid specified in the chosen sequence is non-conservatively substituted, provided that no more than 15% of the amino acid residues in the sequence are so changed. Protein sequences having the indicated number of non-conservative substitutions can be identified using commonly available bioinformatic tools (Mulder N J and Apweiler R, 2002; Rehm B H, 2001).

In addition to such sequences, a series of polypeptides forms part of the disclosure of the invention. Being mucin-like polypeptides known to go through maturation processes including the proteolytic removal of N-terminal sequences (by signal peptidases and other proteolytic enzymes), the present application also claims the mature forms of the polypeptide whose sequence is recited in SEQ ID NO: 3 and/or SEQ ID NO: 7. The sequence of this polypeptide is recited in SEQ ID NO: 4 and/or SEQ ID NO: 8. Mature forms are intended to include any polypeptide showing mucin-like activity and resulting from in vivo (by the expressing cells or animals) or in vitro (by modifying the purified polypeptides with specific enzymes) post-translational maturation processes. Other alternative mature forms can also result from the addition of chemical groups such as sugars or phosphates. The present application also claims the histidine tagged forms forms of the polypeptide whose sequence is recited in SEQ ID NO: 3 and/or SEQ ID NO: 7. The sequence of this polypeptide is recited in SEQ ID NO: 5 and/or SEQ ID NO: 9.

Other claimed polypeptides are the active variants of the amino acid sequences given by SEQ ID NO: 2, SEQ ID NO: 3, SEQ ID NO: 4. SEQ ID NO: 5, SEQ ID NO: 7, SEQ ID NO, 8 or SEQ ID NO: 9, wherein any amino acid specified in the chosen sequence is non-conservatively substituted, provided that no more than 15%, preferably no more that 10%, 5%, 3%, or 1%, of the amino acid residues in the sequence are so changed. The indicated percentage has to be measured over the novel amino acid sequences disclosed.

In accordance with the present invention, any substitution should be preferably a “conservative” or “safe” substitution, which is commonly defined a substitution introducing an amino acids having sufficiently similar chemical properties (e.g. a basic, positively charged amino acid should be replaced by another basic, positively charged amino acid), in order to preserve the structure and the biological function of the molecule.

The literature provide many models on which the selection of conservative amino acids substitutions can be performed on the basis of statistical and physicochemical studies on the sequence and/or the structure of proteins (Rogov S I and Nekrasov A N, 2001). Protein design experiments have shown that the use of specific subsets of amino acids can produce foldable and active proteins, helping in the classification of amino a id “synonymous” substitutions which can be more easily accommodated in protein structure, and which can be used to detect functional and structural homologs and paralogs (Murphy L R et al., 2000). The groups of synonymous amino acids and the groups of more preferred synonymous amino acids are shown in Table I.

Active variants having comparable, or even improved, activity with respect of corresponding mucin-like polypeptides may result from conventional mutagenesis technique of the encoding DNA, from combinatorial technologies at the level of encoding DNA sequence (such as DNA shuffling, phage display/selection), or from computer-aided design studies, followed by the validation for the desired activities as described in the prior art.

Specific, non-conservative mutations can be also introduced in the polypeptides of the invention with different purposes. Mutations reducing the affinity of the mucin-like polypeptide may increase its ability to be reused and recycled, potentially increasing its therapeutic potency (Robinson C R, 2002). Immunogenic epitopes eventually present in the polypeptides of the invention can be exploited for developing vaccines (Stevanovic S, 2002), or eliminated by modifying their sequence following known methods for selecting mutations for increasing protein stability, and correcting them (van den Burg B and Eijsink V, 2002; WO 02/05146, WO 00/34317, WO 98/52976).

Further alternative polypeptides of the invention are active fragments, precursors, salts, or functionally-equivalent derivatives of the amino acid sequences described above.

Fragments should present deletions of terminal or internal amino acids not altering their function, and should involve generally a few amino acids, e.g., under ten, and preferably under three, without removing or displacing amino acids which are critical to the functional conformation of the proteins. Small fragments may form an antigenic determinant.

The “precursors” are compounds which can be converted into the compounds of present invention by metabolic and enzymatic processing prior or after the administration to the cells or to the body.

The term “salts” herein refers to both salts of carboxyl groups and to acid addition salts of amino groups of the polypeptides of the present invention. Salts of a carboxyl group may be formed by means known in the art and include inorganic salts, for example, sodium, calcium, ammonium, ferric or zinc salts, and the like, and salts with organic bases as those formed, for example, with amines, such as triethanolamine, arginine or lysine, piperidine, procaine and the like. Acid addition salts include, for example, salts with mineral acids such as, for example, hydrochloric acid or sulfuric acid, and salts with organic acids such as, for example, acetic acid or oxalic acid. Any of such salts should have substantially similar activity to the peptides and polypeptides of the invention or their analogs.

The term “derivatives” as herein used refers to derivatives which can be prepared from the functional groups present on the lateral chains of the amino acid moieties or on the amino- or carboxy-terminal groups according to known methods. Such molecules can result also from other modifications which do not normally alter primary sequence, for example in vivo or in vitro chemical derivativization of polypeptides (acetylation or carboxylation), those made by modifying the pattern of phosphorylation (introduction of phosphotyrosine, phosphoserine, or phosphothreonine residues) or glycosylation (by exposing the polypeptide to mammalian glycosylating enzymes) of a peptide during its synthesis and processing or in further processing steps. Alternatively, derivatives may include esters or aliphatic amides of the carboxyl groups and N-acyl derivatives of free amino groups or O-acyl derivatives of free hydroxyl-groups and are formed with acyl-groups as for example alcanoyl- or aryl-groups.

The generation of the derivatives may involve a site-directed modification of an appropriate residue, in an internal or terminal position. The residues used for attachment should they have a side-chain amenable for polymer attachment (i.e., the side chain of an amino acid bearing a functional group, e.g., lysine, aspartic acid, glutamic acid, cysteine, histidine, etc.). Alternatively, a residue having a side chain amenable for polymer attachment can replace an amino acid of the polypeptide, or can be added in an internal or terminal position of the polypeptide. Also, the side chains of the genetically encoded amino acids can be chemically modified for polymer attachment, or unnatural amino acids with appropriate side chain functional groups can be employed. The preferred method of attachment employs a combination of peptide synthesis and chemical ligation. Advantageously, the attachment of a water-soluble polymer will be through a biodegradable linker, especially at the amino-terminal region of a protein. Such modification acts to provide the protein in a precursor (or “pro-drug”) form, that, upon degradation of the linker releases the protein without polymer modification.

Polymer attachment may be not only to the side chain of the amino acid naturally occurring in a specific position of the antagonist or to the side chain of a natural or unnatural amino acid that replaces the amino acid naturally occurring in a specific position of the antagonist but also to a carbohydrate or other moiety that is attached to the side chain of the amino acid at the target position. Rare or unnatural amino acids can be also introduced by expressing the protein in specifically engineered bacterial strains (Bock A, 2001).

All the above indicated variants can be natural, being identified in organisms other than humans, or artificial, being prepared by chemical synthesis, by site-directed mutagenesis techniques, or any other known technique suitable thereof, which provide a finite set of substantially corresponding mutated or shortened peptides or polypeptides which can be routinely obtained and tested by one of ordinary skill in the art using the teachings presented in the prior art.

The novel amino acid sequences disclosed in the present patent application can be used to provide different kind of reagents and molecules. Examples of these compounds are binding proteins or antibodies that can be identified using their fun sequence or specific fragments, such as antigenic determinants. Peptide libraries can be used in known methods (Tribbick G, 2002) for screening and characterizing antibodies or other proteins binding the claimed amino acid sequences, and for identifying alternative forms of the polypeptides of the invention having similar binding properties.

The present patent application discloses also fusion proteins comprising any of the polypeptides described above. These polypeptides should contain protein sequence heterologous to the one disclosed in the present patent application, without significantly impairing the mucin-like activity of the polypeptide and possibly providing additional properties. Examples of such properties are an easier purification procedure, a longer lasting half-life in body fluids, an additional binding moiety, the maturation by means of an endoproteolytic digestion, or extracellular localization. This latter feature is of particular importance for defining a specific group of fusion or chimeric proteins included in the above definition since it allows the claimed molecules to be localized in the space where not only isolation and purification of these polypeptides is facilitated, but also where generally mucin-like polypeptides and their receptor interact.

Design of the moieties, ligands, and linkers, as well methods and strategies for the construction, purification, detection and use of fusion proteins are disclosed in the literature (Nilsson J et al., 1997; Methods Enzymol, Vol. 326-328, Academic Press, 2000). The preferred one or more protein sequences which can be comprised in the fusion proteins belong to these protein sequences: membrane bound protein, immunoglobulin constant region, multimerization domains, extracellular proteins, signal peptide-containing proteins, export signal-containing proteins. Features of these sequences and their specific uses are disclosed in a detailed manner, for example, for albumin fusion proteins (WO 01/77137), fusion proteins including multimerization domain (WO 01/02440, WO 00/24782), immunoconjugates (Garnett M C, 2001), or fusion protein providing additional sequences which can be used for purifying the recombinant products by affinity chromatography (Constans A, 2002; Burgess R R and Thompson N E, 2002; Lowe C R et al., 2001; J. Bioch. Biophy. Meth., vol. 49 (1-3), 2001; Sheibani N, 1999).

The polypeptides of the invention can be used to generate and characterize ligands binding specifically to them. These molecules can be natural or artificial, very different from the chemical point of view (binding proteins, antibodies, molecularly imprinted polymers), and can be produced by applying the teachings in the art (WO 02/74938; Kuroiwa Y et al., 2002; Haupt K, 2002; van Dijk M A and van de Winkel J G, 2001; Gavilondo J V and Larrick J W, 2000). Such ligands can antagonize or inhibit the mucin-like activity of the polypeptide against which they have been generated. In particular, common and efficient ligands are represented by extracellular domain of a membrane-bound protein or antibodies, which can be in the form monoclonal, polyclonal, humanized antibody, or an antigen binding fragment.

The polypeptides and the polypeptide-based derived reagents described above can be in alternative forms, according to the desired method of use and/or production, such as active conjugates or complexes with a molecule chosen amongst radioactive labels, fluorescent labels, biotin, or cytotoxic agents.

Specific molecules, such as peptide mimetics, can be also designed on the sequence and/or the structure of a polypeptide of the invention. Peptide mimetics (also called peptidomimetics) are peptides chemically modified at the level of amino acid side chains, of amino acid chirality, and/or of the peptide backbone. These alterations are intended to provide agonists or antagonists of the polypeptides of the invention with improved preparation, potency and/or pharmacokinetics features.

For example, when the peptide is susceptible to cleavage by peptidases following injection into the subject is a problem, replacement of a particularly sensitive peptide bond with a non-cleavable peptide mimetic can provide a peptide more stable and thus more useful as a therapeutic. Similarly, the replacement of an L-amino acid residue is a standard way of rendering the peptide less sensitive to proteolysis, and finally more similar to organic compounds other than peptides. Also useful are amino-terminal blocking groups such as t-butyloxycarbonyl, acetyl, theyl, succinyl, methoxysuccinyl, suberyl, adipyl, azelayl, dansyl, benzyloxycarbonyl, fluorenylmethoxycarbonyl, methoxyazelayl, methoxyadipyl, methoxysuberyl, and 2,4-dinitrophenyl. Many other modifications providing increased potency, prolonged activity, easiness of purification, and/or increased half-life are disclosed in the prior art (WO 02/10195; Villain M et al., 2001).

Preferred alternative, synonymous groups for amino acids derivatives included in peptide mimetics are those defined in Table II. A non-exhaustive list of amino acid derivatives also include aminoisobutyric acid (Aib), hydroxyproline (Hyp), 1,2,3,4-tetrahydro-4-isoquinoline-3-COOH, indoline-2carboxylic acid, 4-difluoro-proline, L-thiazolidine-4-carboxylic acid, L-homoproline, 3,4-dehydro-proline, 3,4-dihydroxy-phenylalanine, cyclohexyl-glycine, and phenylglycine.

By “amino acid derivative” is intended an amino acid or amino acid-like chemical entity other than one of the 20 genetically encoded naturally occurring amino acids. In particular, the amino acid derivative may contain substituted or non-substituted, linear, branched, or cyclic alkyl moieties, and may include one or more heteroatoms. The amino acid derivatives can be made de novo or obtained from commercial sources (Calbiochem-Novabiochem AG, Switzerland; Bachem, USA).

Various methodologies for incorporating unnatural amino acids derivatives into proteins, using both in vitro and in vivo translation systems, to probe and/or improve protein structure and function are disclosed in the literature (Dougherty D A, 2000). Techniques for the synthesis and the development of peptide mimetics, as well as non-peptide mimetics, are also well known in the art (Golebiowski A et al., 2001; Hruby V J and Balse P M, 2000; Sawyer T K, in “Structure Based Drug Design” edited by Veerapandian P, Marcel Dekker Inc, pg. 557-663, 1997).

Another object of the present invention are isolated nucleic acids encoding for the polypeptides of the invention having mucin-like activity, the polypeptides binding to an antibody or a binding protein generated against them, the corresponding fusion proteins, or mutants having antagonistic activity as disclosed above. Preferably, these nucleic acids should comprise a DNA sequence selected from the group consisting of SEQ ID NO: 1 and SEQ ID NO: 6, or the complement of said DNA sequences.

Alternatively, the nucleic acids of the invention should hybridize under high stringency conditions, or exhibit at least about 85% identity over a stretch of at least about 30 nucleotides, with a nucleic acid consisting of SEQ ID NO: 1 and/or SEQ ID NO: 6, or be a complement of said DNA sequence.

The wording “high stringency conditions” refers to conditions in a hybridization reaction that facilitate the association of very similar molecules and consist in the overnight incubation at 60-65° C. in a solution comprising 50% formamide, 5×SSC (150 m M NaCl, 15 m M trisodium citrate), 50 mM sodium phosphate (pH 7.6), 5× Denhardt's solution, 10% dextran sulphate, and 20 microgram/ml denatured, sheared salmon sperm DNA, followed by washing the filters in 0.1×SSC at the same temperature.

These nucleic acids, including nucleotide sequences substantially the same, can be comprised in plasmids, vectors and any other DNA construct which can be used for maintaining, modifying, introducing, or expressing the encoding polypeptide. In particular, vectors wherein said nucleic acid molecule is operatively linked to expression control sequences can allow expression in prokaryotic or eukaryotic host cells of the encoded polypeptide.

The wording “nucleotide sequences substantially the same” includes all other nucleic acid sequences which, by virtue of the degeneracy of the genetic code, also code for the given amino acid sequences. In this sense, the literature provides indications on preferred or optimized codons for recombinant expression (Kane J F et al. 1995).

The nucleic acids and the vectors can be introduced into cells with different purposes, generating transgenic cells and organisms. A process for producing cells capable of expressing a polypeptide of the invention comprises genetically engineering cells with such vectors and nucleic acids.

In particular, host cells (e.g. bacterial cells) can be modified by transformation for allowing the transient or stable expression of the polypeptides encoded by the nucleic acids and the vectors of the invention. Alternatively, said molecules can be used to generate transgenic animal cells or non-human animals (by non-/homologous recombination or by any other method allowing their stable integration and maintenance), having enhanced or reduced expression levels of the polypeptides of the invention, when the level is compared with the normal expression levels. Such precise modifications can be obtained by making use of the nucleic acids of the inventions and of technologies associated, for example, to gene therapy (Meth. Enzymol., vol. 346, 2002) or to site-specific recombinases (Kolb A F, 2002). Model systems based on the mucin-like polypeptides disclosed in the present patent application for the systematic study of their function can be also generated by gene targeting into human cell lines (Bunz F, 2002).

Gene silencing approaches may also be undertaken to down-regulate endogenous expression of a gene encoding a polypeptide of the invention. RNA interference (RNAI) (Elbashir, S M et al., Nature 2001, 411, 494-498) is one method of sequence specific post-transcriptional gene silencing that may be employed. Short dsRNA oligonucleotides are synthesised in vivo and introduced into a cell. The sequence specific binding of these dsRNA oligonucleotides triggers the degradation of target mRNA, reducing or ablating target protein expression.

Efficacy of the gene silencing approaches assessed above may be assessed through the measurement of polypeptide expression (for example, by Western blotting), and at the RNA level using TaqMan-based methodologies.

The polypeptides of the invention can be prepared by any method known in the art, including recombinant DNA-related technologies, and chemical synthesis technologies. In particular, a method for making a polypeptide of the invention may comprise culturing a host or transgenic cell as described above under conditions in which the nucleic acid or vector is expressed, and recovering the polypeptide encoded by said nucleic acid or vector from the culture. For example, when the vector expresses the polypeptide as a fusion protein with an extracellular or signal-peptide containing proteins, the recombinant product can be secreted in the extracellular space, and can be more easily collected and purified from cultured cells in view of further processing or, alternatively, the cells can be directly used or administered.

The DNA sequence coding for the proteins of the invention can be inserted and ligated into a suitable episomal or non-/homologously integrating vectors, which can be introduced in the appropriate host cells by any suitable means (transformation, transfection, conjugation, protoplast fusion, electroporation, calcium phosphate-precipitation, direct microinjection, etc.). Factors of importance in selecting a particular plasmid or viral vector include: the ease with which recipient cells that contain the vector, may be recognized and selected from those recipient cells which do not contain the vector; the number of copies of the vector which are desired in a particular host; and whether it is desirable to be able to “shuttle” the vector between host cells of different species.

The vectors should allow the expression of the isolated or fusion protein including the polypeptide of the invention in the Prokaryotic or Eukaryotic host cells under the control of transcriptional initiation/termination regulatory sequences, which are chosen to be constitutively active or inducible in said cell. A cell line substantially enriched in such cells can be then isolated to provide a stable cell line.

For Eukaryotic hosts (e.g. yeasts, insect plant, or mammalian cells), different transcriptional and translational regulatory sequences may be employed, depending on the nature of the host. They may be derived form viral sources, such as adenovirus, bovine papilloma virus, Simian virus or the like, where the regulatory signals are associated with a particular gene which has a high level of expression. Examples are the TK promoter of the Herpes virus, the SV40 early promoter, the yeast gal4 gene promoter, etc. Transcriptional initiation regulatory signals may be selected which allow for repression and activation, so that expression of the genes can be modulated. The cells stably transformed by the introduced DNA can be selected by introducing one or more markers allowing the selection of host cells which contain the expression vector. The marker may also provide for prototrophy to an auxotropic host biocide resistance, e.g. antibiotics, or heavy metals such as copper, or the like. The selectable marker gene can either be directly linked to the DNA gene sequences to be expressed, or introduced into the same cell by co-transfection.

Host cells may be either prokaryotic or eukaryotic. Preferred are eukaryotic hosts, e.g. mammalian cells, such as human, monkey, mouse, and Chinese Hamster Ovary (CHO) cells, because they provide post-translational modifications to proteins, including correct folding and glycosylation. Also yeast cells can carry out post-translational peptide modifications including glycosylation. A number of recombinant DNA strategies exist which utilize strong promoter sequences and high copy number of plasmids which can be utilized for production of the desired proteins in yeast. Yeast recognizes leader sequences in cloned mammalian gene products and secretes peptides bearing leader sequences (i.e., pre-peptides).

The above mentioned embodiments of the invention can be achieved by combining the disclosure provided by the present patent application on the sequence of novel mucin-like polypeptides with the knowledge of common molecular biology techniques.

Many books and reviews provides teachings on how to clone and produce recombinant proteins using vectors and Prokaryotic or Eukaryotic host cells, such as some titles in the series “A Practical Approach” published by Oxford University Press (“DNA Cloning 2: Expression Systems”, 1995; “DNA Cloning 4: Mammalian Systems”, 1996; “Protein Expression”, 1999; “Protein Purification Techniques”, 2001).

Moreover, updated and more focused literature provides an overview of the technologies for expressing polypeptides in a high-throughput manner (Chambers S P, 2002; Coleman T A, et al., 1997), of the cell systems and the processes used industrially for the large-scale production of recombinant proteins having therapeutic applications (Andersen D C and Krummen L, 2002, Chu L and Robinson D K, 2001), and of alternative eukaryotic expression systems for expressing the polypeptide of interest, which may have considerable potential for the economic production of the desired protein, such the ones based on transgenic plants (Giddings G, 2001) or the yeast Pichia pastoris (Lin Cereghino G P et al., 2002). Recombinant protein products can be rapidly monitored with various analytical technologies during purification to verify the amount and the quantity of the expressed polypeptides (Baker K N et al., 2002), as well as to check if there is problem of bioequivalence and immunogenicity (Schellekens H, 2002; Gendel S M, 2002).

Totally synthetic mucin-like polypeptides are disclosed in the literature and many examples of chemical synthesis technologies, which can be effectively applied for the mucin-like polypeptides of the invention given their short length, are available in the literature, as solid phase or liquid phase synthesis technologies. For example, the amino acid corresponding to the carboxy-terminus of the peptide to be synthesized is bound to a support which is insoluble in organic solvents, and by alternate repetition of reactions, one wherein amino acids with their amino groups and side chain functional groups protected with appropriate protective groups are condensed one by one in order from the carboxy-terminus to the amino-terminus, and one where the amino acids bound to the resin or the protective group of the amino groups of the peptides are released, the peptide chain is thus extended in this manner. Solid phase synthesis methods are largely classified by the tBoc method and the Fmoc method, depending on the type of protective group used. Typically used protective groups include tBoc (t-butoxycarbonyl), Cl-Z (2-chlorobenzyloxycarbonyl), Br-Z (2-bromobenzyloxycarbonyl), Bzl (benzyl), Fmoc (9-fluorenylmethoxycarbonyl), Mbh (4,4′-dimethoxydibenzhydryl), Mtr (4-methoxy-2,3,6-trimethylbenzenesulphonyl), Trt (trityl), Tos (tosyl), Z (benzyloxycarbonyl) and Cl2-Bzl (2,6-dichlorobenzyl) for the amino groups; NO2 (nitro) and Pmc (2,2,5,7,8-pentamethylchromane-6-sulphonyl) for the guanidino groups); and tBu (t-butyl) for the hydroxyl groups). After synthesis of the desired peptide, it is subjected to the de-protection reaction and cut out from the solid support. Such peptide cutting reaction may be carried with hydrogen fluoride or tri-fluoromethane sulfonic acid for the Boc method, and with TFA for the Fmoc method.

The purification of the polypeptides of the invention can be carried out by any one of the methods known for this purpose, i.e. any conventional procedure involving extraction, precipitation, chromatography, electrophoresis, or the like. A further purification procedure that may be used in preference for purifying the protein of the invention is affinity chromatography using monoclonal antibodies or affinity groups, which bind the target protein and which are produced and immobilized on a ge I matrix contained within a column. Impure preparations containing the proteins are passed through the column. The protein will be bound to the column by heparin or by the specific antibody while the impurities will pass through. After washing, the protein is eluted from the gel by a change in pH or ionic strength. Alternatively, HPLC (High Performance Liquid Chromatography) can be used. The elution can be carried using a water-acetonitrile-based solvent commonly employed for protein purification.

The disclosure of the novel polypeptides of the invention, and the reagents disclosed in connection to them (antibodies, nucleic acids, cells) allows also to screen and characterize compounds that enhance or reduce their expression level into a cell or in an animal.

“Oligonucleotides” refers to either a single stranded polydeoxynucleotide or two complementary polydeoxynucleotide strands which may be chemically synthesized. Such synthetic oligonucleotides have no 5′ phosphate and thus will not ligate to another oligonucleotide without adding a phosphate with an ATP in the presence of a kinase. A synthetic oligonucleotide will ligate to a fragment that has not been dephosphorylated.

The invention includes purified preparations of the compounds of the invention (polypeptides, nucleic acids, cells, etc.). Purified preparations, as used herein, refers to the preparations which contain at least 1%, preferably at least 5%, by dry weight of the compounds of the invention.

Therapeutic Uses

The present patent application discloses a series of novel mucin-like polypeptides and of related reagents having several possible applications. In particular, whenever an increase in the mucin-like activity of a polypeptide of the invention is desirable in the therapy or in the prevention of a disease, reagents such as the disclosed mucin-like polypeptides, the corresponding fusion proteins and peptide mimetics, the encoding nucleic acids, the expressing cells, or the compounds enhancing their expression can be used.

Therefore, the present invention discloses pharmaceutical compositions for the treatment or prevention of diseases needing an increase in the mucin-like activity of a polypeptide of the invention, which contain one of the disclosed mucin-like polypeptides, the corresponding fusion proteins and peptide mimetics, the encoding nucleic acids, the expressing cells, or the compounds enhancing their expression, as active ingredient. The process for the preparation of these pharmaceutical compositions comprises combining the disclosed mucin-like polypeptides, the corresponding fusion proteins and peptide mimetics, the encoding nucleic acids, the expressing cells, or the compounds enhancing their expression, together with a pharmaceutically acceptable carrier. Methods for the treatment or prevention of diseases needing an increase in the mucin-like activity of a polypeptide of the invention, comprise the administration of a therapeutically effective amount of the disclosed mucin-like polypeptides, the corresponding fusion proteins and peptide mimetics, the encoding nucleic acids, the expressing cells, or the compounds enhancing their expression.

Amongst the reagents disclosed in the present patent application, the ligands, the antagonists or the compounds reducing the expression or the activity of polypeptides of the invention have several applications, and in particular they can be used in the therapy or in the diagnosis of a disease associated to the excessive mucin-like activity of a polypeptide of the invention.

Therefore, the present invention discloses pharmaceutical compositions for the treatment or prevention of diseases associated to the excessive mucin-like activity of a polypeptide of the invention, which contain one of the ligands, antagonists, or compounds reducing the expression or the activity of such polypeptides, as active ingredient. The process for the preparation of these pharmaceutical compositions comprises combining the ligand, the antagonist, or the compound, together with a pharmaceutically acceptable carrier. Methods for the treatment or prevention of diseases associated to the excessive mucin-like activity of the polypeptide of the invention, comprise the administration of a therapeutically effective amount of the antagonist, the ligand or of the compound.

SCS0004 and/or SCS0005 nucleic acid molecules, polypeptides, and agonists and antagonists thereof can be used to treat, diagnose, ameliorate, or prevent a number of diseases, disorders, or conditions, including those recited herein.

SCS0004 and/or SCS0005 polypeptide agonists and antagonists include those molecules which regulate SCS0004 and/or SCS0005 polypeptide activity and either increase or decrease at least one activity of the mature form of the SCS0004 and/or SCS0005 polypeptide. Agonists or antagonists may be co-factors, such as a protein, peptide, carbohydrate, lipid, or small molecular weight molecule, which interact with SCS0004 and/or SCS0005 polypeptide and thereby regulate its activity.

Potential polypeptide agonists or antagonists include antibodies that react with either soluble or membrane-bound forms of SCS0004 and/or SCS0005 polypeptides that comprise part or all of the extracellular domains of the said proteins. Molecules that regulate SCS0004 and/or SCS0005 polypeptide expression typically include nucleic acids encoding SCS0004 and/or SCS0005 polypeptide that can act as anti-sense regulators of expression.

SCS0004 and SCS0004 variant were determined to be splice variants of MUC6, whereas SCS0005 a splice variant of MUC5AC (Example 2). MUC5AC and MUC6 have already been involved in many diseases (see hereafter). As such, SCS0004 SCS0004 variant and SCS0005 nucleic acid molecules, polypeptides, and agonists and antagonists thereof may be useful in diagnosing or treating those diseases.

Mucin glycoproteins are a major macromolecular component of mucus. Mucins are large, heavily glycosylated glycoproteins that are expressed in two major forms: the membrane-tethered mucins and the secreted mucins. In the airways, MUC1 and MUC4 are the predominant membrane-tethered mucins that are present on epithelial cell surfaces; MUC5AC, MUC5B and MUC2 are the predominant secreted mucins that contribute to the mucus gel (Voynow J A. Paediatr Respir Rev. 2002 June; 3(2): 98-103. What does mucin have to do with lung disease?).

Mata et al. showed that the numbers of mucus secretory cells in airway epithelium, and the Muc5ac messenger ribonucleic acid and protein expression, were markedly augmented in rats exposed to bleomycin and that these changes were significantly reduced in NAC (N-acetylcysteine)-treated rats (Mata et al. Eur Respir J. 2003 December; 22(6): 900-5. Oral N-acetylcysteine reduces bleomycin-induced lung damage and mucin Muc5ac expression in rats). They add that these results indicate that bleomycin increases the number of airway secretary cells and their mucin production, and that oral N-acetylcysteine improves pulmonary lesions and reduced the mucus hypersecretion in the bleomycin rat model of pulmonary fibrosis. Furthermore, airway mucins (including MUC5AC) are oversulfated in cystic fibrosis as well as in chronic bronchitis, and this feature has been considered as being linked to a primary defect of these diseases (Lamblin et al. Glycoconj J. 2001 September; 18(9): 661-84. Human airway mucin glycosylation: a combinatory of carbohydrate determinants which vary in cystic fibrosis. See also hereafter). Overexpression of MUC5AC, MUC5B and MUC2 correlates strongly with secretory cell hyperplasia and metaplasia in human and murine airways. Harris A, suggests that MUC6 is also implicated in cystic fibrosis as a significant component of the material that obstructs the pancreatic ducts. (Harris A Ann N Y Acad Sci. 1999 Jun. 30; 880: 17-30. The duct cell in cystic fibrosis). As such SCS0004 and/or SCS0005 nucleic acid molecules, polypeptides, and agonists and preferably antagonists (e.g. antibodies) thereof may be useful in diagnosing or treating cystic fibrosis, pulmonary fibrosis, and bronchitis and/or prevent secretory cell hyperplasia and metaplasia in human and murine airways.

Matsuzwa et al suggest that the up-regulation of the expression of gastric gland mucous cells (GMC) mucins, of which MUC6 (a core protein of GMC Mucins), may be involved in defense against Helicobacter pylori infection in the gastric surface mucous gel layer and on the gastric mucosa (Matsuzwa et al. Helicobacter. 2003 December; 8(6): 594-600. Helicobacter pylori infection up-regulates gland mucous cell-type mucins in gastric pyloric mucosa). Van De Bovenkamp et al. showed that gastric metaplasia of the duodenum (GMD) is characterized by the expression of MUC5AC and MUC6 with a probable role of role H. pylori in GMD development (Van De Bovenkamp et al. Hum Pathol. 2003 February; 34(2): 156-65. Metaplasia of the duodenum shows a Helicobacter pylori-correlated differentiation into gastric-type protein expression). In addition, Byrd et al. showed that H. pylori inhibits total mucin synthesis in vitro and decreases the expression of MUC5AC and MUC1 (Byrd et al. Gastroenterology. 2000 June; 118(6): 1072-9. Inhibition of gastric mucin synthesis by Helicobacter pylori). They add that a decrease in gastric mucin synthesis in vivo may disrupt the protective surface mucin layer. In addition, Mathoera et al. showed that membrane mucin expression (including MUC5AC) was correlated with relative antibiotic resistance (Mathoera et al. Infect Immun. 2002 December; 70(12): 7022-32. Pathological and therapeutic significance of cellular invasion by Proteus mirabilis in an enterocystoplasty infection stone model). They showed that all cell lines showed colocalization of Proteus mirabilis with human colonic mucin (i.e., MUC2) and human gastric mucin (i.e., MUC5AC). They state that bacterial invasion seems to have cell type-dependent mechanisms and prolong bacterial survival in antibiotic therapy, giving a new target for therapeutic optimalization of antibiotic treatment. Furthermore, Nutten et al. suggest that mucin genes (including MUC5AC) have abilities to protect epithelial cells against Shigella flexneri (Nutten et al. Microbes Infect 2002 September; 4(11): 1121-4. Epithelial inflammation response induced by Shigella flexneri depends on mucin gene expression). As such SCS0004 and/or SCS0005 nucleic acid molecules, polypeptides, and agonists thereof may be useful in preventing bacterial infection (e.g. Proteus mirabilis, Helicobacter pylori, Helicobacter heilmannii, Pseudomonas aeruginosa, Shigella flexneri).

Airway mucins from severely infected patients suffering either from cystic fibrosis or from chronic bronchitis are also highly sialylated, and highly express sialylated and sulfated Lewis x determinants, a feature which may reflect severe mucosal inflammation or infection. These determinants are also potential sites of attachment for Pseudomonas aeruginosa, the pathogen responsible for most of the morbidity and mortality in cystic fibrosis. Helicobacter pylori binding to human gastric mucins is also strain- and blood-group dependent in contrast, binding to human gastric mucins at acidic pH seems to be a common feature for all H. pylori strains that is independent of the expression of blood group structures on host mucins (Linden et al. Biochem J. 2004 Jan. 21; Pt. [Epub ahead of print] Rhesus monkey gastric mucins: Oligomeric structure, glycoforms and Helicobacter pylori binding). The Le^{b} blood group antigen has been shown to mediate attachment of H. pylori to the human gastric mucosa and the MUC5AC mucin, whereas sialylated Lewis antigens contribute to binding in inflamed tissue (Linden et al.). In addition, correlation between binding of the BabA positive H. pylori strain to carbohydrate were found to the Le^{b}/fucosylated structures (stronger correlation for MUC5AC than MUC6, still Linden et al.). As such, SCS0004 and/or SCS0005 antagonists (e.g. antibodies targeted to SCS0004 and/or SCS0005) and specifically antagonists to glycosylation sites, preferably sulfation sites, preferably sialylated sites, myristoylation sites, amidation sites, glycosaminoglycan attachment sites, mannosylation sites, or preferably fucosilation sites of SCS0004 and/or SCS0005 or other molecules that can reduce sialylation or sulfation of SCS0004 and/or SCS0005 (indicated in part in example 3) may be useful in preventing attachment of various bacterial species to SCS0004 and/or SCS0005, or reducing antibiotic resistance. These bacterial species include Helicobacter pylori, Helicobacter heilmannii (which are both responsible for the loss of mucus and the cause of gastric and duodenal ulcers as well as gastric cancer, gastritis), Pseudomonas aeruginosa, Proteus mirabillis, and Shigella flexneri.

Takeyama et al. showed that cigarette smoke inhalation increased MUC5AC mRNA and goblet cell production in rat airways in vivo, effects that were prevented by pretreatment with BIBX1522. They add that these effects may explain the goblet cell hyperplasia that occurs in chronic obstructive pulmonary disease (COPD) and may provide a novel strategy for therapy in airway hypersecretory diseases (Takeyama et al. Am J Physiol Lung Cell Mol Physiol. 2001 January; 280(1): L165-72. Activation of epidermal growth factor receptors is responsible for mucin synthesis induced by cigarette smoke). As such SCS0004 and/or SCS0005 nucleic acid molecules, polypeptides, and agonists and preferably antagonists (e.g. antibodies) thereof may be useful in diagnosing or treating chronic obstructive pulmonary disease (COPD), airway hypersecretory diseases, preventing or treating goblet cell hyperplasia and diminishing deletious effects of cigarette smoke.

Shahzeidi et al state that in murine models of allergic asthma (Goblet cell hyperplasia (GCH) is a characteristic of asthma), mice repeatedly exposed to allergens or interleukin (IL)13 have numerous goblet cells in their airway epithelium, in contrast to healthy naive mice (Shahzeidi et al Exp Lung Res. 2003 December; 29(8): 549-65. Temporal analysis of goblet cells and mucin gene expression in murine models of allergic asthma.). They showed that increased Muc5ac and Muc2 mRNA expression occurred following ovalbumin or IL13 exposure and that Muc5ac protein was expressed in so me goblet transition and goblet cells. Studies by Song et al. give additional insights into the molecular mechanism of IL-1beta- and TNF-alpha-induced MUC5AC gene expression and of the mucin hypersecretion during inflammation (Song et al. J Biol Chem. 2003 Jun. 27; 278(26): 23243-50. Epub 2003 Apr. 10. Interleukin-1 beta and tumor necrosis factor-alpha induce MUC5AC overexpression through a mechanism involving ERK/p38 mitogen-activated protein kinases-MSK1-CREB activation in human airway epithelial cells). Miller et al. state that severe inflammation and mucus overproduction are partially responsible for respiratory syncytial virus (RSV)-induced disease in infants (Miller et al. J Immunol. 2003 Mar. 15; 170(6): 3348-56. CXCR2 regulates respiratory syncytial virus-induced airway hyperreactivity and mucus overproduction). They showed that CXCR2(−/−) mice displayed a statistically significant decrease in muc5ac, relative to RSV-infected wild-type animals. They further state that CXCR2 may be a relevant target in the pathogenesis of RSV bronchiolitis. MUC5AC is also expressed in allergic rhinitis (Voynow et al. Lung. 1998; 176(5): 345-54. Mucin gene expression (MUC1, MUC2, and MUC515AC) in nasal epithelial cells of cystic fibrosis, allergic rhinitis, and normal individuals). In addition, the results presented by Kaneko et al. suggest that overproduction of muc5ac plays an important role in the pathogenesis of diffuse panbronchiolitis (DPB) and that clinical improvement following macrolide therapy seems to involve, at least in part, its inhibition of mucin overproduction, through modulation of intracellular signal transduction (Kaneko et al. Am J Physiol Lung Cell Mol Physiol. 2003 October; 285(4): L847-53. Epub 2003 Jun. 20. Clarithromycin inhibits overproduction of muc5ac core protein in murine model of diffuse panbronchiolitis).

Gray et al suggest that the synchronous regulation of ASL mucin and liquid metabolism triggered by IL-1 beta may be an important defense mechanism of the airway epithelium to enhance mucociliary clearance during airway inflammation (Gray et al., Am J Physiol Lung Cell Mol Physiol. 2004 February; 286(2): L320-L330. Epub 2003 Oct. 3. Regulation of MUC5AC mucin secretion and airway surface liquid metabolism by IL-1{beta} in human bronchial epithelia.). They showed that IL-1beta, in a dose- and time-dependent manner, increased the secretion of MUC5AC, but not MUC5B. Findings of Kunert et al. demonstrate that, in the conjunctiva of mice, repetitive application of allergens (mouse model of allergic conjunctivitis) induces a reduction in the number of filled goblet cells and a decrease in Muc5AC and Muc4 mRNAs (Kunert et al. Invest Ophthalmol Vis Sci. 2001 October; 42(11): 2483-9. Alteration in goblet cell numbers and mucin gene expression in a mouse model of allergic conjunctivitis). As such SCS0004 and/or SCS0005 nucleic acid molecules, polypeptides, and agonists and preferably antagonists (e.g. antibodies) thereof may be useful in diagnosing or treating allergic asthma, inflammation (e.g. airway inflammation), respiratory syncytial virus (RSV)-induced disease, RSV bronchiolitis, allergic rhinitis or panbronchiolitis (DPB), allergic conjunctivitis, or in enhancing or reducing mucociliary clearance.

Capper et al. showed that otitis media with effusion (OME) is characterized by the accumulation of a viscous fluid rich in mucins, of which MUC5AC and MUC6, in the middle ear cleft (Clin Otolaryngol. 2003 February; 28(1): 51-4. Effect of nitric oxide donation on mucin production in vitro: Takeuchi at al. Int J Pediatr Otorhinolaryngol. 2003 January; 67(1): 53-8. Mucin gene expression in the effusions of otitis media with effusion.). As such SCS0004 and/or SCS0005 nucleic acid molecules, polypeptides, and agonists and preferably antagonists (e.g. antibodies) thereof may be useful in diagnosing or treating otitis (e.g. otitis media with effusion (OME)).

Paulsen et al. showed that human efferent tear ducts express and produce a broad spectrum of mucins (including MUC6 and MUC5AC) that is partly comparable with that in the conjunctiva and the salivary glands (Paulsen et al. Invest Ophthalmol Vis Sci. 2003 May, 44(5): 1807-13. Characterization of mucins in human lacrimal sac and nasolacrimal duct). They add that the mucin diversity of the efferent tear ducts could enhance tear transport and antimicrobial defense thereby easing tear flow. In addition, Argueso et al. propose that deficiency of MUC5AC mucin in tears constitutes one of the mechanisms responsible for tear film instability in Sjogren syndrome (Argueso et al. Invest Ophthalmol Vis Sci. 2002 April; 43(4): 1004-11. Decreased levels of the goblet cell mucin MUC5AC in tears of patients with Sjogren syndrome). As such SCS0004 and/or SCS0005 nucleic acid molecules, polypeptides, and agonists thereof may be useful in diagnosing or treating Sjogren syndrome, enhancing tear transport and antimicrobial defense, easing tear flow or in reducing tear film instability.

Aarbiou et al. showed that HNP1-3 (human neutrophil peptides 1-3 [HNP1-3]) increased mRNA encoding the mucins MUC5B and MUC5AC, suggesting a role for defensins in mucous cell differentiation (Aarbiou et al. Am J Respir Cell Mol Biol. 2004 February; 30(2): 193-201. Epub 2003 Jul. 18. Neutrophil defensins enhance lung epithelial wound closure and mucin gene expression in vitro.). They add that their results indicate that neutrophil defensins increase epithelial wound repair in vitro important in case of tissue injury, which involves migration and proliferation, and mucin production. Results provided by Buisine et al suggest that gel forming mucins (more particularly MUC5AC and MUC6) may have a role in epithelial wound healing after mucosal injury in inflammatory bowel diseases such as Crohn's disease (CD) in addition to mucosal protection (Buisine et al. Gut 2001 October; 49(4): 544-1. Mucin gene expression in intestinal epithelial cells in Crohn's disease). As such, SCS0005 nucleic acid molecules, polypeptides, and agonists thereof may be useful in diagnosing, treating or reducing tissue injury (e.g. mucosal injury), epithelial wounding, inflammatory bowel diseases such as Crohn's disease (CD), or in increasing epithelial wound repair or in procuring mucosal protection.

Mall et al. state that Menetier's disease is a rare gastric condition characterized by marked proliferation of the mucosa and variable mucus secretion and achlorhydria, adding as well that stomachs stained positively for MUC4, 5AC and 6, which are typically found in gastric mucosa (Mail et al. J Gastroenterol Hepatol. 2003 July; 18(7): 876-9. Expression of gastric mucin in the stomachs of two patients with Menetrier's disease: an immunohistochemical study). As such SCS0004 and/or SCS0005 nucleic acid molecules, polypeptides, and agonists and preferably antagonists (e.g. antibodies) thereof may be useful in diagnosing or treating achlorhydria or Menetrier's disease.

Jonckheere et al showed that exogenous addition of TGF-beta to epithelial cancer cells induces Muc5ac endogenous expression (Jonckheere et al. Biochem J. 2004 Feb. 1; 377(Pt 3): 797-808. Transcriptional activation of the murine Muc5ac mucin gene in epithelial cancer cells by TGF-beta/Smad4 signalling pathway is potentiated by Sp1). In addition, Li et al showed that over-expression of SOX2, a SRY-related HMG box protein, induced the mRNA expression of endogenous MUC5AC in COS-7 cells (Int J Oncol. 2004 February; 24(2): 257-63. Expression of the SRY-related HMG box protein SOX2 in human gastric carcinoma). They add that these findings indicate that SOX2 may play a role in differentiation of the human gastric epithelium, and that SOX2 may be involved in gastric carcinogenesis, particularly in the gastric type. Mitsuhashi et al showed that absence of MUC5AC expression seems correlated with worse survival in patients with adenocarcinoma of the uterine cervix (Mitsuhashi et al. Ann Surg Oncol. 2004 January; 11(1): 40-4. Correlation between MUC5AC expression and the prognosis of patients with adenocarcinoma of the uterine cervix). MUC5AC's expression was also observed in pancreatic tumors or pancreatic ductal adenocarcinomas (Yamasaki et al. Int J Oncol. 2004 January; 24(1): 107-13. Expression and localization of MUC1. MUC2. MUC5AC and small intestinal mucin antigen in pancreatic tumors; Iacobuzio-Donahue et al. Cancer Res. 2003 Dec. 15; 63(24): 8614-22. Highly expressed genes in pancreatic ductal adenocarcinomas: a comprehensive characterization and comparison of the transcription profiles obtained from three major technologies.), in nasal epithelial cells (Choi et al. Acta Otolaryngol. 2003 December; 123(9): 1080-6. Uridine-5-triphosphate and adenosine triphosphate gammaS induce mucin secretion via Ca2+-dependent pathways in human nasal epithelial cells), in hepatobiliary cystadenoma and cystadenocarcinoma of the gall bladder (Terada et al. Pathol Int 2003 November; 53(11): 790-5. Hepatobiliary cystadenocarcinoma with cystadenoma elements of the gall bladder in an old man), in cholangiocarcinoma (Boonla et al. Cancer. 2003 Oct. 1; 98(7): 1438-43. Prognostic value of serum MUC5AC mucin in patients with cholangiocarcinoma), in invasive breast cancer tissues (Vgenopoulou et al. Breast. 2003 June; 12(3): 172-8. Immunohistochemical evaluation of immune response in invasive ductal breast cancer of not-otherwise-specified type), in cholangiocarcinoma tissues (Wongkham et al. Cancer Lett. 2003 May 30; 195(1): 93-9. Serum MUC5AC mucin as a potential marker for cholangiocarcinoma), in colorectal cancer (Bara et al. Tumour Biol. 2003 May-June; 24(3): 109-15. Abnormal expression of gastric mucin in human and rat aberrant crypt foci during colon carcinogenesis), in binary papillo matosis (Amaya et al. Histopathology. 2001 June; 38(6): 550-60. Expression of MUC1 and MUC2 and carbohydrate antigen Tn change during malignant transformation of binary papillomatosis), in chronic ethmoiditis mucosa (Jung et al. Am J Rhinol. 2000 May-June; 14(3): 163-70. Expression of mucin genes in chronic ethmoiditis), and in rectosigmoid villous adenoma (Buisine et al. Gastroenterology. 1996 January; 110(1): 84-91. Aberrant expression of a human mucin gene (MUC5AC) in rectosigmoid villous adenoma). In addition, Kocer et al. showed that absence of MUC5AC expression in tumors can be a prognostic factor for more aggressive colorectal carcinoma (Kocer et al. Pathol Int 2002 July; 52(7): 470-7. Expression of MUC5AC in colorectal carcinoma and relationship with prognosis). As such, SCS0005 nucleic acid molecules, polypeptides, and agonists and preferably antagonists (e.g. antibodies) thereof may be useful in diagnosing or treating epithelial cancer, gastric carcinoma, gastric and duodenal ulcers, gastric cancer, gastritis, adenocarcinoma of the uterine cervix, pancreatic tumors or pancreatic ductal adenocarcinomas, nasal epithelial cells, hepatobiliary cystadenoma and cystadenocarcinoma of the gall bladder, cholangiocarcinoma, colorectal cancer, billary papiliomatosis, chronic ethmoiditis mucosa and rectosigmoid villous adenoma. Enss et al. demonstrated differential cytokine effects on mucin synthesis, secretion and composition. They add that these alterations may contribute to the defective mucus layer in colitis (Enss et al. Inflamm Res. 2000 April 49(4): 162-9. Proinflammatory cytokines trigger MUC gene expression and mucin release in the intestinal cancer cell line LS180). As such SCS0004 and/or SCS0005 nucleic acid molecules, polypeptides, and agonists and preferably antagonists (e.g. antibodies) thereof may be useful in diagnosing or treating colitis.

The results presented by Nishiumi. et al. suggest that 11p15 mucins MUC2 and MUC6 are related to lymph node metastasis in small adenocarcinoma of the lung (SACL; Nishiumi et al. Clin Cancer Res. 2003 Nov. 15; 9(15): 5616-9. Use of 11p15 mucins as prognostic factors in small adenocarcinoma of the lung). In addition, Perrais et al. showed that MUC2 and MUC5AC are two target genes of epidermal growth factor receptor (EGFR) ligands in lung cancer cells (Perrais at al. J Biol Chem. 2002 Aug. 30; 277(35): 32258-67. Epub 2002 Jun. 19. Induction of MUC2 and MUC5AC mucins by factors of the epidermal growth factor (EGF) family is mediated by EGF receptor/Ras/Raf/extracellular signal-regulated kinase cascade and Sp1). As such SCS0004 and/or SCS0005 nucleic acid molecules, polypeptides, and agonists and preferably antagonists (e.g. antibodies) thereof may be useful in diagnosing or treating small adenocarcinoma of the lung, or lung cancer or prevent lymph node metastasis. MUC5AC's immunoreactivity was observed in Barrett's esophagus and gastric intestinal metaplasia (Piazuelo et al. Mod Pathol. 2004 January; 17(1): 62-74. Phenotypic differences between esophageal and gastric intestinal metaplasia), in human colon carcinomas (Truant et al. Int J Cancer. 2003 May 10; 104(6): 683-94. Requirement of both mucins and proteoglycans in cell-cell dissociation and invasiveness of colon carcinoma HT-29 cells), in ovarian mucinous tumourigenesis and primary ovarian carcinoma (Boman et al. J Pathol. 2001 March, 193(3): 339-44. Mucin gene transcripts in benign and borderline mucinous tumours of the ovary: an in situ hybridization study), in chronic cholecystitis (Ho et al. Dig Dis Sol. 2000 June, 45(6): 1061-71. Altered mucin core peptide expression in acute and chronic cholecystitis). As such, SCS0005 nucleic acid molecules, polypeptides, and agonists and preferably antagonists (e.g. antibodies) thereof may be useful in diagnosing or treating Barrett's esophagus and gastric intestinal metaplasia, colon carcinomas, ovarian mucinous tumourigenesis and primary ovarian carcinoma and chronic cholecystitis.

Yoshii et al. showed that the decrease or loss of MUC5AC expression may have an important role in the invasive growth of Paget cells involved in Extramammary Paget's disease (EPD), which is a relatively common skin cancer wherein tumor cells have mucin in their cytoplasm (Yoshii et al. Pathol Int 2002 May-June; 52(5-6): 390-9. Expression of mucin core proteins in extramammary Paget's disease). As such, SCS0005 nucleic acid molecules, polypeptides, and agonists and antagonists (e.g. antibodies) thereof may be useful in diagnosing or treating skin cancer, Extramammary Paget's disease (EPD), or in preventing invasive growth of Paget cells.

Tsukamoto et al. showed that MUC5AC and MUC6 transcripts decreased with the progression of intestinal metaplasia (Tsukamoto et al. J Cancer Res Clin Oncol. 2003 Dec. 4 Down-regulation of a gastric transcription factor, Sox2, and ectopic expression of intestinal homeobox genes, Cdx1 and Cdx2: inverse correlation during progression from gastric/intestinal-mixed to complete intestinal metaplasia). As such SCS0004 and/or SCS0005 nucleic acid molecules, polypeptides, and agonists and preferably antagonists (e.g. antibodies) thereof may be useful in diagnosing or treating intestinal metaplasia.

Gallbladder mucins play a critical role in the pathogenesis of cholesterol gallstones because of their ability to bind biliary lipids and accelerate cholesterol crystallization (Wang et al. J Lipid Res. 2004 Jan. 1. Targeted disruption of the murine mucin gene 1 decreases susceptibility to cholesterol gallstone formation). Wang et al. showed that the gene expression of the gallbladder Muc1 and Muc5ac was significantly reduced in Muc1−/− mice in response to a lithogenic diet. In addition, Lee et al. showed that altered mucin gene expression was found in gallbladders with cholesterol stones and calcium bilirubinate stones, as evidenced by the presence of MUC2 and MUC4 and the increased expression of MUC1, MUC3, MUC5B and MUC6 (Lee et al. J Formos Med Assoc. 2002 November: 101(11): 762-8. Mucin gene expression in gallbladder epithelium). Expression of MUC5AC (in carcinoma) and MUC6 on dysplasia or non-dysplastic epithelia) was detected in the gallbladder (Sasaki et al. Pathol Int 1999 January; 49(1): 38-44. Expression of MUC2, MUC5AC and MUC6 apomucins in carcinoma, dysplasia and non-dysplastic epithelia of the gallbladder). Furthermore, chronic proliferative cholangitis, characterized by an active and long-standing inflammation of the stone-containing bile ducts (intrahepatic calculi) with the hyperplasia of epithelia and the proliferation of the duct-associated mucus glands, displayed an increase in mRNA levels of cystic fibrosis transmembrane conductance regulator (CFTR) as well as MUC2, MUC3, MUC5AC, MUC5B, and MUC6 in affected ducts compared with the ducts from control subjects, reflecting the increased amounts of total biliary mucins (Shoda et al. Hepatology. 1999 April: 29(4): 1026-36. Secretory low-molecular-weight phospholipases A2 and their specific receptor in bile ducts of patients with intrahepatic calculi: factors of chronic proliferative cholangitis). In addition, Zen et al. suggest that lipopolysaccharide (LPS) can induce overexpression of MUC2 and MUC5AC in binary epithelial cells via synthesis of TNF-alpha and activation of protein kinase C. This mechanism might be involved in the lithogenesis of hepatolithiasis (Zen et al Am J Pathol. 2002 October; 161(4): 1475-84. Lipopolysaccharide induces overexpression of MUC2 and MUC5AC in cultured biliary epithelial cells: possible key phenomenon of hepatolithiasis). As such SCS0004 and/or SCS0005 nucleic acid molecules, polypeptides, and agonists and preferably antagonists (e.g. antibodies) thereof may be useful in diagnosing or treating hepatolithiasis or preventing lithogenesis.

As such SCS0004 and/or SCS0005 nucleic acid molecules, polypeptides, and agonists and preferably antagonists (e.g. antibodies) thereof may be useful in the clearance of cholesterol gallstones, calcium bilirubinate stones, intrahepatic calculi, in preventing lithogenesis and in diagnosing or treating chronic proliferative cholangitis or carcinoma, hepatolithiasis, dysplasia and non-dysplastic epithelia of the gallbladder.

Recognizing that the air pollutant residual oil fly ash (ROFA) constituent vanadium is a potent tyrosine phosphatase inhibitor and that mucin induction by pathogens is phophotyrosine dependent, Longphre et al. suggest that vanadium-containing air pollutants trigger disease-like conditions by unmasking phosphorylation dependent pathogen resistance pathways (Longphre et al. Toxicol Appl Pharmacol. 2000 Jan. 15; 162(2): 86-92. Lung mucin production is stimulated by the air pollutant residual oil fly ash). As such SCS0004 and/or SCS0005 nucleic acid molecules, polypeptides, and agonists and preferably antagonists (e.g. antibodies) thereof may be useful in diagnosing or treating air pollutant related diseases (e.g. ROFA related diseases).

In addition to the above, MUC5AC is highly expressed in the following libraries according to the Unigene MUC5AC entry (http://www.ncbl.nlm.nih.gov/UniGene/clust.cgi?ORG=Hs&CID=103707: Ascites; adenocarcinoma; colon; head-normal; olfactory epithelium; head-neck moderately-differentiated adenocarcinoma; breast-normal; adenocarcinoma cell line; lung_tumor; pooled colon, kidney, stomach; two pooled squamous cell card nomas; Purified pancreatic islet; cervix; stomach-normal; colon-normal; Stomach colon_est; normal head/neck tissue; poorly differentiated adenocarcinoma with signet ring cell features; squamous cell carcinoma, poorly differentiated (4 pooled tumors, including primary and metastatic); prostate_normal colon tumor, RER+; pooled; breast; stomach; poorly-differentiated endometrial adenocarcinoma, 2 pooled tumors; Primary Lung Cystic Fibrosis Epithelial Cells; pancreas; Human Lung Epithelial cells; colon tumor; well-differentiated endometrial adenocarcinoma, 7 pooled tumors colonic mucosa from 5 ulcerative colitis patients; colon tumor RER+; colonic mucosa from 3 patients with Crohn's disease; ovary; B-ell, chronic lymphotic leukemia; adenocarcinoma, cell line; trachea. As such SCS0005 nucleic acid molecules, polypeptides, and agonists and antagonists (e.g. antibodies) thereof may be useful in diagnosing or treating diseases related to the above organs or tissues, as well as the above-mentioned diseases or cancers.

The results presented by Leroy et al implicate human mucin genes (MUC1, MUC3, and MUC6) in renal morphogenesis processes such as fetal kidney development and malformed cystic renal diseases (Leroy et al. Am J Clin Pathol. 2003 October; 120(4): 544-50. Expression of human mucin genes during normal and abnormal renal development). As such SCS0004 nucleic acid molecules, polypeptides, and agonists and antagonists thereof may be useful in diagnosing or treating malformed cystic renal diseases, and in renal morphogenesis processes such as fetal kidney development.

Leroy et al further state that MUC6 is a valuable marker of seminal vesicle-ejaculatory duct and is useful for the differential diagnosis with prostate adenocarcinoma (Leroy et al. Am J Surg Pathol. 2003 April, 27(4): 519-21. MUC6 is a marker of seminal vesicle-ejaculatory duct epithelium and is useful for the differential diagnosis with prostate adenocarcinoma). As such SCS0004 nucleic acid molecules, polypeptides, and agonists and preferably antagonists (e.g. antibodies) thereof may be useful in diagnosing or treating prostate adenocarcinoma.

MUC6 is expressed in normal and tumour kidney (Leroy et al. Histopathology. 2002 May, 40(5): 450-7. Expression of human mucin genes in normal kidney and renal cell carcinoma) in primary liver cancer (Sasaki et al. Pathol Int. 1999 April, 49(4): 325-31. Expression of sialyl-Tn, Tn and T antigens in primary liver cancer), in pancreatic and bile duct adenocarcinomas (Bartman et al. J Pathol. 1998 Dec. 186(4): 398-405. The MUC6 secretory mucin gene is expressed in a wide variety of epithelial tissues), in breast cancers (de Bolos et al. Int J Cancer. 1998 Jul. 17; 77(2): 193-9. MUC6 expression in breast tissues and cultured cells: abnormal expression in tumors and regulation by steroid hormones), in chronic viral hepatitis (Sasaki et al. J Pathol. 1998 June; 185(2): 191-8. Increased MUC6 apomucin expression is a characteristic of reactive billary epithelium in chronic viral hepatitis). As such SCS0004 nucleic acid molecules, polypeptides, and agonists and preferably antagonists (e.g. antibodies) thereof may be useful in diagnosing or treating tumour kidney, in primary liver cancer, in pancreatic and bile duct adenocarcinomas, breast cancers, or chronic viral hepatitis. Expression of the MUC2, MUC3, MUC5AC and MUC6 genes was demonstrated in ovarian mucinous tumor, occurrence of which is favored by Peutz-Jeghers syndrome (Wacrenier et al. PJS, Ann Pathol. 1998 December; 18(6): 497-501). As such SCS0004 and/or SCS0005 nucleic acid molecules, polypeptides, and agonists and preferably antagonists (e.g. antibodies) thereof may be useful in diagnosing or treating ovarian mucinous tumor or Peutz-Jeghers syndrome.

In addition to the above, MUC6 is highly expressed in the following libraries according to the Unigene MUC6 entry (http://www.ncbl.nlm.nih.gov/UniGene/clust.cgi?ORG=Hs&CID=398100): Stomach; colon; lung_normal; nervous_normal; head_neck; lobullar carcinoma in situ; prostate_normal; breast; colon_normal; stomach_normal; prostate; stomach; normal prostate; adenocarcinoma; poorly differentiated adenocarcinoma with signet ring cell features: Ascites; well-differentiated endometrial adenocarcinoma, 7 pooled tumors; nervous_tumor; insulinoma. As such SCS0004 nucleic acid molecules, polypeptides, and agonists and antagonists (e.g. antibodies) thereof may be useful in diagnosing or treating diseases related to the above organs or tissues, as well as the above-mentioned diseases or cancers.

Without wishing to be bound to theory, the von Willebrand factor (vWF) type D and C domains found in SCS0004, SCS0004 variant and SCS0005 (Example 3) are likely to be involved in the formation of multiprotein complexes (a common feature of von Willebrand factor type D and C containing proteins). In addition, expression of VWF containing proteins can occur after induction by growth factors or certain oncogenes. As such, antagonists (e.g. antibodies) directed to the SCS0004's and/or SCS0005's von Willebrand factor type D and C domains or one or more of its four distinct modules may be useful in hindering von Willebrand factor type D and C multimers or complex formation, thereby disrupting surface mucous gel layer or mucosa, and useful in diagnosing or treating the above mentioned cancers or diseases where antagonists of SCS0004 and/or SCS0005 are preferably used. Agonists (e.g. antibodies) directed to the SCS0004's and/or SCS0005's von Willebrand factor type D and C domains or one or more of its four distinct modules may be useful in diagnosing or treating the above mentioned diseases where agonists of SCS0004 and/or SCS0005 are preferably used (e.g. Sjogren syndrome, enhancing tear transport and antimicrobial defense, easing tear flow or reduce tear film instability, tissue injury (e.g. mucosal injury), epithelial wounding, inflammatory bowel diseases such as Crohn's disease (CD), or increasing epithelial wound repair or procure mucosal protection).

Without wishing to be bound to theory, antagonists (e.g. antibodies) directed to the SCS0004's and/or SCS0005's trypsin inhibitor like cysteine rich domains, WAP-type domains or cystine-knot domains (Example 3) may disrupt disulphide formations and interfere with the proper folding of the proteins of the invention. In addition, the WAP-type domain might be involved in the metastatic potential of carcinomas. As such, antagonists (e.g. antibodies) directed to the SCS0004's and/or SCS0005's trypsin inhibitor like cysteine rich domains, WAP-type or cystine-knot domains may be useful in diagnosing or treating the above mentioned cancers or diseases where antagonists of SCS0004 and/or SCS0005 are preferably used. Agonists (e.g. antibodies) directed to the SCS0004's and/or SCS0005's trypsin inhibitor like cysteine rich domains, WAP-type domains or cystine-knot domains may be useful in diagnosing or treating the above mentioned diseases where agonists of SCS0004 and/or SCS0005 are preferably used (e.g. Sjogren syndrome, enhancing tear transport and antimicrobial defense, easing tear flow or reduce tear film instability, tissue injury (e.g. mucosal injury), epithelial wounding, inflammatory bowel diseases such as Crohn's disease (CD), or increasing epithelial wound repair or procure mucosal protection).

Without wishing to be bound to theory, antagonists (e.g. antibodies) directed to the SCS0004's and/or SCS0005's zinc binding domains (Example 3) may disrupt the zing fingers and dimer formation, thereby interfering with its responsive elements and subsequent transcriptions of the proteins of the invention. The function of zinc fingers in the estrogen receptor DNA-binding domain (DBD) was shown to be susceptible to chemical inhibition by electrophilic disulfide benzamide and benzisothiazolone derivatives, which selectively block binding of the estrogen receptor to its responsive element and subsequent transcription (Wang et al. Nat Med. 2004 January; 10(1):40-47. Epub 2003 Dec. 14. Suppression of breast cancer by chemical modulation of vulnerable zinc fingers in estrogen receptor). Wang et al. acid that these compounds also significantly inhibit estrogen-stimulated cell proliferation, markedly reduce tumor mass in nude mice bearing human MCF-7 breast cancer xenografts, and interfere with cell-cycle and apoptosis regulatory gene expression. As such, antagonists (e.g. antibodies) or electrophilic disulfide benzamide and benzisothiazolone derivatives directed to the SCS0004's and/or SCS0005's zinc binding domains may be useful in diagnosing or treating the above mentioned cancers or diseases where antagonists of SCS0004 and/or SCS0005 are preferably used. Agonists (e.g. antibodies) directed to the SCS0004's and/or SCS0005's zinc binding domains may be useful in diagnosing or treating the above mentioned diseases where agonists of SCS0004 and/or SCS0005 are preferably used (e.g. Sjogren syndrome, enhancing tear transport and antimicrobial defense, easing tear flow or reduce tear film instability, tissue injury (e.g. mucosal injury), epithelial wounding, inflammatory bowel diseases such as Crohn's disease (CD), or increasing epithelial wound repair or procure mucosal protection).

Without wishing to be bound to theory, antagonists (e.g. antibodies) directed to the SCS0004's and/or SCS0005's PCSK (only in SCS0004 variant, motif is KRC) or NDR cleavage sites (Example 3) might interfere with the processing of the latent proteins precursors of the invention into their biologically active products. Paired basic amino acid cleaving system 4 (SPC4 or PACE4) and furin are serine endoproteases that have for substrate, among others, the von Willebrand factor. As such, antagonists (e.g. antibodies) directed to the SCS0004's and/or SCS0005's PCSK (KRC motif of SCS0004) or NDR cleavage sites may be useful in diagnosing or treating the above mentioned cancers or diseases where antagonists of SCS0004 and/or SCS0005 are preferably used. Agonists (e.g. antibodies) directed to the SCS0004's and/or SCS0005's PCSK (KRC motif of SCS0004) or NDR cleavage sites may be useful in diagnosing or treating the above mentioned diseases where agonists of SCS0004 and/or SCS0005 are preferably used (e.g. Sjogren syndrome, enhancing tear transport and antimicrobial defense, easing tear flow or reduce tear film instability, tissue injury (e.g. mucosal injury), epithelial wounding, inflammatory bowel diseases such as Crohn's disease (CD), or increasing epithelial wound repair or procure mucosal protection).

Without wishing to be bound to theory, antagonists (e.g. antibodies) directed to the SCS0005's RGD integrin binding site (Example 3) might disrupt heterodimers formation of alpha and beta subunits and interfere with proper ligand binding. RGD sequences have been found to be responsible for the cell adhesive properties of a number of proteins, including von Willebrand factor. As such, antagonists (e.g. antibodies) directed to the SCS0005's RGD integrin binding site may be useful in diagnosing or treating the above mentioned cancers or diseases where antagonists of SCS0005 are preferably used. Agonists (e.g. antibodies) directed to the SCS0005's RGD integrin binding site may be useful in diagnosing or treating the above mentioned diseases where agonists of SCS0005 are preferably used (e.g. Sjogren syndrome, enhancing tear transport and antimicrobial defense, easing tear flow or reduce tear film instability, tissue injury (e.g. mucosal injury), epithelial wounding, inflammatory bowel diseases such as Crohn's disease (CD), or increasing epithelial wound repair or procure mucosal protection).

Without wishing to be bound to theory, antagonists (e.g. antibodies) directed to the SCS0004's and/or SCS0005's SH2 domains, Polo-like domains, cAMP- and cGMP-dependent protein kinase phosphorylation sites, Protein kinase C phosphorylation sites, Casein kinase II phosphorylation sites, Tyrosine kinase phosphorylation sites (Example 3) might interfere with signaling pathways (proper propagation of signal downstream) and disrupting protein-protein interaction and/or modifying enzymatic activities. As such, antagonists (e.g. antibodies) directed to the SCS0004's and/or SCS0005's SH2 domains, Polo-like domains, cAMP- and cGMP-dependent protein kinase phosphorylation sites, Protein kinase C phosphorylation sites, Casein kinase II phosphorylation sites, Tyrosine kinase phosphorylation sites may be useful in diagnosing or treating the above mentioned cancers or diseases where antagonists of SCS0004 and/or SCS0005 are preferably used. Agonists (e.g. antibodies) directed to the SCS0004's and/or SCS0005's SH2 domains, Polo-like domains, cAMP- and cGMP-dependent protein kinase phosphorylation sites, Protein kinase C phosphorylation sites, Casein kinase II phosphorylation sites, Tyrosine kinase phosphorylation sites may be useful in diagnosing or treating the above mentioned diseases where agonists of SCS0004 and/or SCS0005 are preferably used (e.g. Sjogren syndrome, enhancing tear transport and antimicrobial defense, easing tear flow or reduce tear film instability, tissue injury (e.g. mucosal injury), epithelial wounding, inflammatory bowel diseases such as Crohn's disease (CD), or increasing epithelial wound repair or procure mucosal protection).

Without wishing to be bound to theory, antagonists (e.g. antibodies) directed to the SCS0004's (WGHW) and/or SCS0005's (WTKW) C-Mannosylation sites, O-Fucosilation sites (CINGRLSC in SCS0004 variant only), N-glycosylation sites, Sulfation sites, N-myristoylation sites, amidation sites (Example 3) might interfere with proper folding of the proteins of the invention. As such, antagonists (e.g. antibodies) directed to the SCS0004's (WGHW) and/or SCS0005's (WTKW) C-Mannosylation sites, O-Fucosilation sites (CINGRLSC in SCS0004 variant only), N-glycosylation sites, Sulfation sites, N-myristoylation sites, amidation sites may be useful in diagnosing or treating the above mentioned cancers or diseases where antagonists of SCS0004 and/or SCS0005 are preferably used. Agonists (e.g. antibodies) directed to the SCS0004's (WGHW) and/or SCS0005's (WTKW) C-Mannosylation sites, O-Fucosilation sites (CINGRLSC in SCS0004 variant only), N-glycosylation sites, Sulfation sites, N-myristoylation sites, amidation sites may be useful in diagnosing or treating the above mentioned diseases where agonists of SCS0004 and/or SCS0005 are preferably used (e.g. Sjogren syndrome, enhancing tear transport and antimicrobial defense, easing tear flow or reduce tear film instability, tissue injury (e.g. mucosal injury), epithelial wounding, inflammatory bowel diseases such as Crohn's disease (CD), or increasing epithelial wound repair or procure mucosal protection).

Without wishing to be bound to theory, antagonists (e.g. antibodies) directed to the SCS0004's and/or SCS0005's glycosaminoglycan attachment sites (Example 3) might interfere with proper cell communication, and interfere in morphogenesis and development. Mutations in some proteoglycans are associated with an inherited predisposition to cancer. As such, antagonists (e.g. antibodies) directed to the SCS0004's and/or SCS0005's glycosaminoglycan attachment sites may be useful in diagnosing or treating the above mentioned cancers or diseases where antagonists of SCS0004 and/or SCS0005 are preferably used. Agonists (e.g. antibodies) directed to the SCS0004's and/or SCS0005's glycosaminoglycan attachment sites may be useful in diagnosing or treating the above mentioned diseases where agonists of SCS0004 and/or SCS0005 are preferably used (e.g. Sjogren syndrome, enhancing tear transport and antimicrobial defense, easing tear flow or reduce tear film instability, tissue injury (e.g. mucosal injury), epithelial wounding, inflammatory bowel diseases such as Crohn's disease (CD), or increasing epithelial wound repair or procure mucosal protection).

The pharmaceutical compositions of the invention may contain, in addition to mucin-like polypeptide or to the related reagent, suitable pharmaceutically acceptable carriers, biologically compatible vehicles and additives which are suitable for administration to an animal (for example, physiological saline) and eventually comprising auxiliaries (like excipients, stabilizers, adjuvants, or diluents) which facilitate the processing of the active compound into preparations which can be used pharmaceutically.

The pharmaceutical compositions may be formulated in any acceptable way to meet the needs of the mode of administration. For example, of biomaterials, sugar-macromolecule conjugates, hydrogels, polyethylene glycol and other natural or synthetic polymers can be used for improving the active ingredients in terms of drug delivery efficacy. Technologies and models to validate a specific mode of administration are disclosed in literature (Davis B G and Robinson M A, 2002; Gupta P et al., 2002; Luo B and Prestwich G D, 2001; Cleland J L et al., 2001; Pillai O and Panchagnula R, 2001).

Polymers suitable for these purposes are biocompatible, namely, they are non-toxic to biological systems, and many such polymers are known. Such polymers may be hydrophobic or hydrophilic in nature, biodegradable, non-biodegradable, or a combination thereof. These polymers include natural polymers (such as collagen, gelatin, cellulose, hyaluronic acid), as well as synthetic polymers (such as polyesters, polyorthoesters, polyanhydrides). Examples of hydrophobic non-degradable polymers include polydimethyl siloxanes, polyurethanes, polytetrafluoroethylenes, polyethylenes, polyvinyl chlorides, and polymethyl methaerylates. Examples of hydrophilic non-degradable polymers include poly(2-hydroxyethyl methacrylate), polyvinyl alcohol, poly(N-vinyl pyrrolidone), polyalkylenes, polyacrylamide, and copolymers thereof. Preferred polymers comprise as a sequential repeat unit ethylene oxide, such as polyethylene glycol (PEG).

Any accepted mode of administration can be used and determined by those skilled in the art to establish the desired blood levels of the active ingredients. For example, administration may be by various parenteral routes such as subcutaneous, intravenous, intradermal, intramuscular, intraperitoneal, intranasal, transdermal, oral, or buccal routes. The pharmaceutical compositions of the present invention can also be administered in sustained or controlled release dosage forms, including depot injections, osmotic pumps, and the like, for the prolonged administration of the polypeptide at a predetermined rate, preferably in unit dosage forms suitable for single administration of precise dosages.

Parenteral administration can be by bolus injection or by gradual perfusion over time. Preparations for parenteral administration include sterile aqueous or non-aqueous solutions, suspensions, and emulsions, which may contain auxiliary agents or excipients known in the art, and can be prepared according to routine methods. In addition, suspension of the active compounds as appropriate oily injection suspensions may be administered. Suitable lipophilic solvents or vehicles include fatty oils, for example, sesame oil, or synthetic fatty acid esters, for example, sesame oil, or synthetic fatty acid esters, for example, ethyl create or triglycerides. Aqueous injection suspensions that may contain substances increasing the viscosity of the suspension include, for example, sodium carboxymethyl cellulose, sorbitol, and/or dextran. Optionally, the suspension may also contain stabilizers. Pharmaceutical compositions include suitable solutions for administration by injection, and contain from about 0.01 to 99.99 percent, preferably from about 20 to 75 percent of active compound together with the excipient.

The wording “therapeutically effective amount” refers to an a mount of the active ingredients that is sufficient to affect the course and the severity of the disease, leading to the reduction or remission of such pathology. The effective amount will depend on the route of administration and the condition of the patient.

The wording “pharmaceutically acceptable” is meant to encompass any carrier, which does not interfere with the effectiveness of the biological activity of the active ingredient and that is not toxic to the host to which is administered. For example, for parenteral administration, the above active ingredients may be formulated in unit dosage form for injection in vehicles such as saline, dextrose solution, serum albumin and Ringers solution. Carriers can be selected also from starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol, and the various oils, including those of petroleum, animal, vegetable or synthetic origin (peanut oil, soybean oil, mineral oil, sesame oil).

It is understood that the dosage administered will be dependent upon the age, sex, health, and weight of the recipient, kind of concurrent treatment, if any, frequency of treatment, and the nature of the effect desired. The dosage will be tailored to the individual subject, as is understood and determinable by one of skill in the art. The total dose required for each treatment may be administered by multiple doses or in a single dose. The pharmaceutical composition of the present invention may be administered alone or in conjunction with other therapeutics directed to the condition, or directed to other symptoms of the condition. Usually a daily dosage of active ingredient is comprised between 0.01 to 100 milligrams per kilogram of body weight per day. Ordinarily 1 to 40 milligrams per kilogram per day given in divided doses or in sustained release form is effective to obtain the desired results. Second or subsequent administrations can be performed at a dosage, which is the same, less than, or greater than the initial or previous dose administered to the individual.

Apart from methods having a therapeutic or a production purpose, several other methods can make use of the mucin-like polypeptides and of the related reagents disclosed in the present patent application.

In a first example, a method is provided for screening candidate compounds effective to treat a disease related to a mucin-like polypeptide of the invention, said method comprising:
(a) contacting host cells expressing such polypeptide, transgenic non-human animals, or transgenic animal cells having enhanced or reduced expression levels of the polypeptide, with a candidate compound and
(b) determining the effect of the compound on the animal or on the cell.

In a second example there is provided a method for identifying a candidate compound as an antagonist/inhibitor or agonist/activator of a polypeptide of the invention, the method comprising:
(a) contacting the polypeptide, the compound, and a mammalian cell or a mammalian cell membrane; and
(b) measuring whether the molecule blocks or enhances the interaction of the polypeptide, or the response that results from such interaction, with the mammalian cell or the mammalian cell membrane.

In a third example, a method for determining the activity and/or the presence of the polypeptide of the invention in a sample, can detect either the polypeptide or the encoding RNA/DNA. Thus, such a method comprises:
(a) providing a protein-containing sample;
(b) contacting said sample with a ligand of the invention; and
(c) determining the presence of said ligand bound to said polypeptide, thereby determining the activity and/or the presence of polypeptide in said sample.

In an alternative, the method comprises:
(a) providing a nucleic acids-containing sample;
(b) contacting said sample with a nucleic acid of the invention; and
(c) determining the hybridization of said nucleic acid with a nucleic acid into the sample, thereby determining the presence of the nucleic acid in the sample.

In this sense, a primer sequence derived from the nucleotide sequence presented in SEQ ID NO: 1 and/or SEQ ID NO: 6 can be used as well for determining the presence or the amount of a transcript or of a nucleic acid encoding a polypeptide of invention in a sample by means of Polymerase Chain Reaction amplification.

A further object of the present invention are kits for measuring the activity and/or the presence of mucin-like polypeptide of the invention in a sample comprising one or more of the reagents disclosed in the present patent application: a mucin-like polypeptide of the invention, an antagonist, ligand or peptide mimetic, an isolated nucleic acid or the vector, a pharmaceutical composition, an expressing cell, or a compound increasing or decreasing the expression levels.

Such kits can be used for in vitro diagnostic or screenings methods, and their actual composition should be adapted to the specific format of the sample (e.g. biological sample tissue from a patient), and the molecular species to be measured. For example, if it is desired to measure the concentration of the mucin-like polypeptide, the kit may contain an antibody and the corresponding protein in a purified form to compare the signal obtained in Western blot. Alternatively, if it is desired to measure the concentration of the transcript for the mucin-like polypeptide, the kit may contain a specific nucleic acid probe designed on the corresponding ORF sequence, or may be in the form of nucleic acid array containing such probe. The kits can be also in the form of protein-, peptide mimetic-, or cell-based microarrays (Templin M F et al., 2002; Pellois J P et al., 2002; Blagoev B and Pandey A. 2001), allowing high-throughput proteomics studies, by making use of the proteins, peptide mimetics and cells disclosed in the present patent application.

The present patent application discloses novel mucin-like polypeptides and a series of related reagents that may be useful, as active ingredients in pharmaceutical compositions appropriately formulated, in the treatment or prevention of diseases and conditions in which mucin-like polypeptides are implicated such as various cancers such as cell proliferative disorders, autoimmune/inflammatory disorders, cardiovascular disorders, neurological disorders, developmental disorders, metabolic disorders, infections and other pathological conditions.

The therapeutic applications of the polypeptides of the invention and of the related reagents can be evaluated (in terms or safety, pharmacokinetics and efficacy) by the means of the in vivo/in vitro assays making use of animal cell, tissues and or by the means of in silico/computational approaches (Johnson D E and Wolfgang G H, 2000), known for the validation of mucin-like polypeptides and other biological products during drug discovery and preclinical development.

The invention will now be described with reference to the specific embodiments by means of the following Examples, which should not be construed as in any way limiting the present invention. The content of the description comprises all modifications and substitutions which can be practiced by a person skilled in the art in light of the above teachings and, therefore, without extending beyond the meaning and purpose of the claims.

TABLE I

| | | More Preferred |
|---|---|---|
| Amino Acid | Synonymous Groups | Synonymous Groups |
| Ser | Gly, Ala, Ser, Thr, Pro | Thr, Ser |
| Arg | Asn, Lys, Gln, Arg, His | Arg, Lys, His |
| Leu | Phe, Ile, Val, Leu, Met | Ile, Val, Leu, Met |
| Pro | Gly, Ala, Ser, Thr, Pro | Pro |
| Thr | Gly, Ala, Ser, Thr, Pro | Thr, Ser |
| Ala | Gly, Thr, Pro, Ala, Ser | Gly, Ala |
| Val | Met, Phe, Ile, Leu, Val | Met, Ile, Val, Leu |
| Gly | Ala, Thr, Pro, Ser, Gly | Gly, Ala |
| Ile | Phe, Ile, Val, Leu, Met | Ile, Val, Leu, Met |
| Phe | Trp, Phe, Tyr | Tyr, Phe |
| Tyr | Trp, Phe, Tyr | Phe, Tyr |
| Cys | Ser, Thr, Cys | Cys |
| His | Asn, Lys, Gln, Arg, His | Arg, Lys, His |
| Gln | Glu, Asn, Asp, Gln | Asn, Gln |
| Asn | Glu, Asn, Asp, Gln | Asn, Gln |
| Lys | Asn, Lys, Gln, Arg, His | Arg, Lys, His |
| Asp | Glu, Asn, Asp, Gln | Asp, Glu |
| Glu | Glu, Asn, Asp, Gln | Asp, Glu |
| Met | Phe, Ile, Val, Leu, Met | Ile, Val, Leu, Met |
| Trp | Trp, Phe, Tyr | Trp |

TABLE II

| Amino | |
|---|---|
| Acid | Synonymous Groups |
| Ser | D-Ser, Thr, D-Thr, allo-Thr, Met, D-Met, Met(O), D-Met(O), |
| | L-Cys, D-Cys |
| Arg | D-Arg, Lys, D-Lys, homo-Arg, D-homo-Arg, Met, Ile, |
| | D-.Met, D-Ile, Orn, D-Orn |
| Leu | D-Leu, Val, D-Val, AdaA, AdaG, Leu, D-Leu, Met, D-Met |
| Pro | D-Pro, L-I-thioazolidine-4-carboxylic acid, D-or L-1- |
| | oxazolidine-4-carboxylic acid |
| Thr | D-Thr, Ser, D-Ser, allo-Thr, Met, D-Met, Met(O), D-Met(O), |
| | Val, D-Val |
| Ala | D-Ala, Gly, Alb, B-Ala, Acp, L-Cys, D-Cys |
| Val | D-Val, Leu, D-Leu, Ile, D-Ile, Met, D-Met, AdaA, AdaG |
| Gly | Ala, D-Ala, Pro, D-Pro, Alb, .beta.-Ala, Acp |
| Ile | D-Ile, Val, D-Val, AdaA, AdaG, Leu, D-Leu, Met, D-Met |
| Phe | D-Phe, Tyr, D-Thr, L-Dopa, His, D-His, Trp, D-Trp, Trans-3,4, |
| | or 5-phenylproline, AdaA, AdaG, cis-3,4, or 5-phenylproline, |
| | Bpa, D-Bpa |
| Tyr | D-Tyr, Phe, D-Phe, L-Dopa, His, D-His |
| Cys | D-Cys, S-Me-Cys, Met, D-Met, Thr, D-Thr |
| Gln | D-Gln, Asn, D-Asn, Glu, D-Glu, Asp, D-Asp |
| Asn | D-Asn, Asp, D-Asp, Glu, D-Glu, Gln, D-Gln |
| Lys | D-Lys, Arg, D-Arg, homo-Arg, D-homo-Arg, Met, D-Met, Ile, |
| | D-Ile, Orn, D-Orn |
| Asp | D-Asp, D-Asn, Asn, Glu, D-Glu, Gln, D-Gln |
| Glu | D-Glu, D-Asp, Asp, Asn, D-Asn, Gln, D-Gln |
| Met | D-Met, S-Me-Cys, Ile, D-Ile, Leu, D-Leu, Val, D-Val |

### EXAMPLES

### Example 1

Sequences of CYS_KNOT protein domains from the ASTRAL database (Brenner S E et al., “The ASTRAL compendium for protein structure and sequence analysis” Nucleic Acids Res. 2000 Jan. 1; 28 (1): 254-6) were used to search for homologous protein sequences in genes predicted from human genome sequence (Celera database). The protein sequences were obtained from the gene predictions and translations thereof as generated by one of three programs: the Genescan (Burge C, Karlin S., “Prediction of complete gene structures in human genomic DNA, J Mol Biol. 1997 Apr. 25; 268(1):78-94) Grail (Xu Y, Uberbacher E C., “Automated gene identification in large-scale genomic sequences”. J Comput Biol. 1997 Fall; 4(3):325-38) and Fgenesh (Proprietary Celera software).

The sequence profiles of the CYS-KNOT domains were generated using PIMAII (Profile Induced Multiple Alignment; Boston University software, version II, Das S and Smith T F 2000), an algorithm that aligns homologous sequences and generates a sequence profile. The homology was detected using PIMAII that generates global-local alignments between a query profile and a hit sequence. In this case the algorithm was used with the profile of the CYS-KNOT functional domain as a query. PIMAII compares the query profile to the database of gene prediction a translated into protein sequence and can therefore identify a match to a DNA sequence that contains that domain. Further comparison by BLAST (Basic Local Alignment Search Tool; NCBI version 2) of the sequence with known CYS-KNOT containing proteins identified the closets homolog (Gish W, States D J. “Identification of protein coding regions by database similarity search.”, Nat Genet. 1993 March; 3(3):266-72; Pearson W R, Miller W., “Dynamic programming algorithms for biological sequence comparison.”, Methods Enzymol. 1992; 210:575-601; Altschul S F et al., “Basic local alignment search tool”, J Mol Biol. 1990 Oct. 5; 215(3):403-10). PIMAII parameters used for the detection were the PIMA prior amino acids probability matrix and a Z-cutoff score of 10. BLAST parameters used were: Comparison matrix=BLOSUM62; word length=3; E value cutoff=10; Gap opening and extension=default; No filter.

Once the functional domain was identified in the sequence, the genes were re-predicted with the genewise algorithm using the sequence of the closest homolog (Bimey E et at, “PairWise and SearchWise: finding the optimal alignment in a simultaneous comparison of a protein profile against all DNA translation frames.”, Nucleic Acids Res. 1996 Jul. 15; 24(14):2730-9).

The profiles for homologous CYS_KNOT domains were generated automatically using the PSI-BLAST (Altshul et al. 1997) scripts written in PERL (Practical Extraction and Report Language) and PIMAII.

A total of 55 predicted genes out of the 464 matching the original query generated on the basis of CYS_KNOT domain profiles were selected.

The novelty of the protein sequences was finally assessed by searching protein databases (SwissProt/Trembl, Human IPI and Derwent GENESEQ) using BLAST and a specific annotation has been attributed on the basis of amino acid sequence homology.

### Example 2

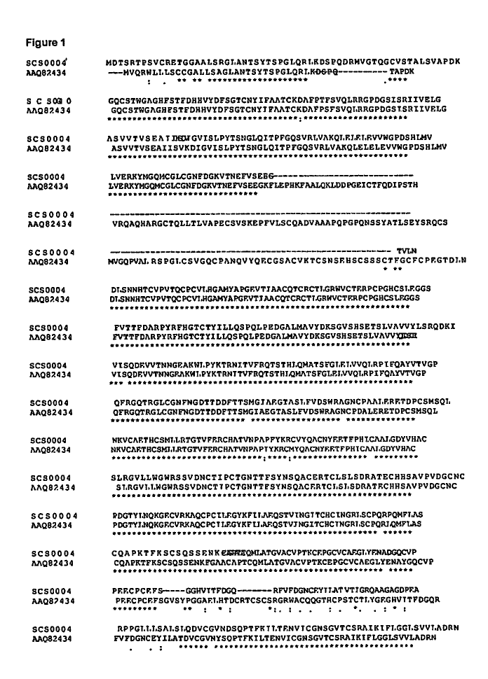

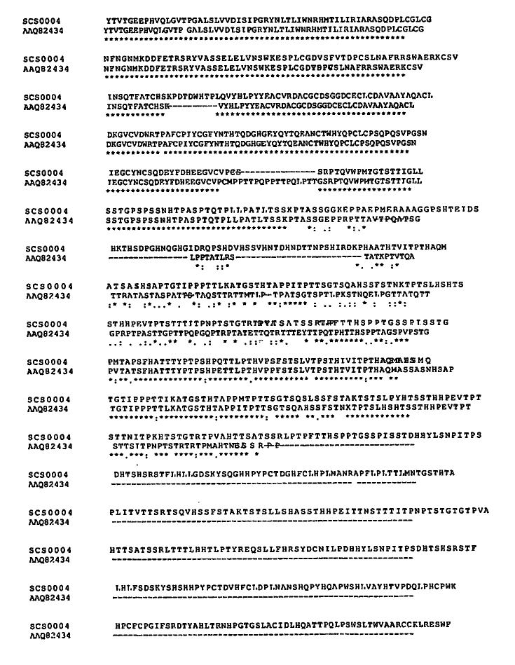

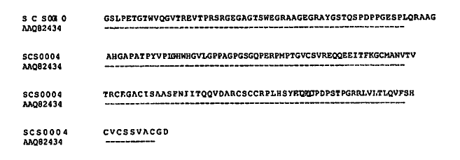

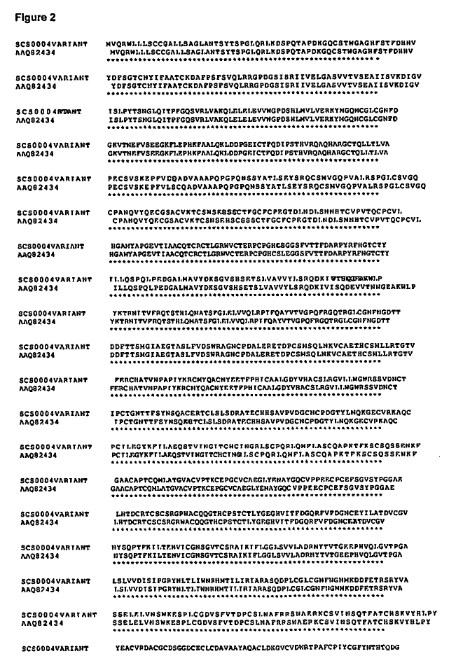

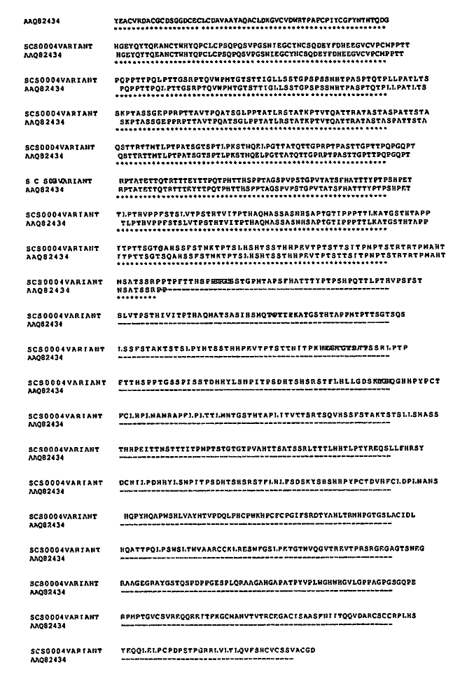

SCS0004 and SCS0004 variants were determined to be splice variants of mucin 6 (MUC6, Homo sapiens, SwissProt entry AAQ82434). SCS0004 is shown to have no signal peptide, whereas SCS0004 variant does. SCS0004 and SCS0004 variant have been shown to align to MUC6 with respectively 71% (FIG. 1) and 100% homology (FIG. 2. AAQ82434 is a fragment of SCS0004 variant).

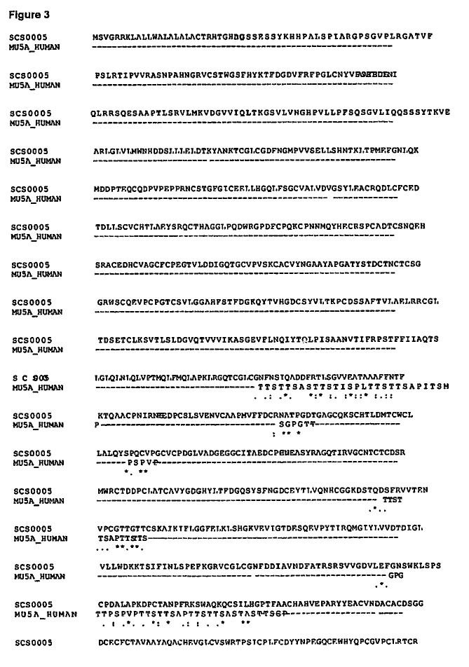

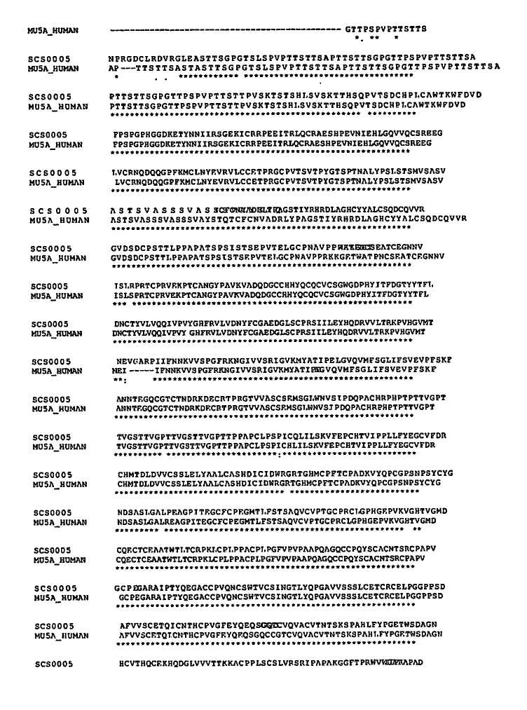

SCS0005 has been shown to have a signal peptide. This protein is predicted to contain four von Willebrand factor D domains, two von Willebrand factor C domains and two trypsin inhibitor domains. This protein aligns to human tracheobronchial mucin MUC5AC with 82% homology over 1056 amino acids (FIG. 3).

### Example 3

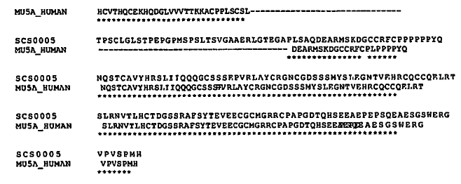

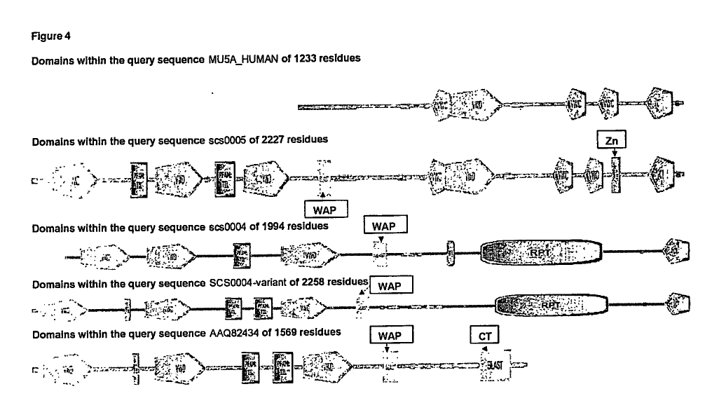

Bioinformatic tools called SMART (http://smart.embl-heidelberg.de/), Prosite (http://us.expasy.org/prosite/, PROSITE Release 18.19, of 17 Jan. 2004) and ELM (http://elm.eu.org/) were used to identify domains and other features of the sequences of the present invention. SMART was used to identify the putative domains of SCS0004, SCS0004 variant and SCS0005. Results of SMART are shown in FIG. 4. Prosite and ELM were not run on SCS0004 (no signal sequence).

SMART Results for SCS0004 variant:
Confidently predicted domains, repeats, motifs and features:

| name | begin | end | E-value |
|---|---|---|---|
| signal peptide | 1 | 18 | — |
| VWD | 33 | 192 | 5.66e−27 |
| ZnF_NFX | 318 | 337 | 0.00e+00 |
| VWC | 358 | 400 | 1.83e+00 |
| VWD | 385 | 548 | 4.39e−33 |
| Pfam: TIL | 663 | 720 | 8.10e−04 |
| Pfam: TIL | 763 | 826 | 4.30e−05 |
| VWC | 828 | 889 | 2.99e+00 |
| VWD | 855 | 1017 | 5.11e−34 |
| low complexity | 1197 | 1212 | — |
| low complexity | 1223 | 1241 | — |
| low complexity | 1244 | 1264 | — |
| low complexity | 1293 | 1338 | — |
| low complexity | 1351 | 1414 | — |
| Internal repeat 1 | 1423 | 1809 | 8.63e−74 |
| Internal repeat 1 | 1592 | 1979 | 8.63e−74 |
| low complexity | 2099 | 2108 | — |
| CT | 2170 | 2257 | 1.16e−29 |

SMART Results for SCS0005:
Confidently predicted domains, repeats, motifs and features:

| name | begin | end | E-value |
|---|---|---|---|
| signal peptide | 1 | 20 | — |
| VWD | 69 | 227 | 2.54e−29 |
| Pfam: TIL | 338 | 394 | 3.10e−11 |
| VWC | 396 | 443 | 2.69e−01 |
| VWD | 423 | 587 | 3.59e−38 |
| low complexity | 591 | 605 | — |
| Pfam: TIL | 625 | 693 | 3.60e−03 |
| VWC | 695 | 737 | 5.23e−01 |
| VWD | 722 | 882 | 1.08e−41 |
| low complexity | 1036 | 1110 | — |
| low complexity | 1250 | 1279 | — |
| low complexity | 1327 | 1344 | — |
| VWC | 1352 | 1417 | 1.26e+00 |
| VWD | 1410 | 1584 | 6.83e−53 |
| low complexity | 1612 | 1650 | — |
| VWC | 1783 | 1849 | 4.61e−18 |
| VWC | 1888 | 1952 | 1.23e−04 |
| ZnF_NFX | 1982 | 2010 | 0.00e+00 |
| CT | 2107 | 2193 | 5.62e−37 |
| low complexity | 2201 | 2214 | — |

Prosite Results for SCS0004 variant:
>PDOC00001 PS00001 ASN_GLYCOSYLATION N-glycosylation site [pattern] [Warning:
pattern with a high probability of occurrence].

| | |
|---|---|
| 21-24 | NTSY |
| 268-271 | NSSY |
| 347-350 | NHTC |
| 485-488 | NJTV |
| 658-661 | NCTI |
| 666-669 | NTTF |
| 901-904 | NYSQ |
| 942-945 | NYTV |
| 974-977 | NLTL |
| 1151-1154 | NCTW |
| 1178-1181 | NCSQ |
| 1475-1478 | NHSA |
| 1869-1872 | NSTT |
| 2185-2188 | NVTV |

>PDOC00003 PS00003 SULFATION Tyrosine sulfation site [rule] [Warning: rule
with a high probability of occurrence].

| | |
|---|---|
| 884-898 | vfdgnceYilatdvc |
| 1137-1151 | tqdghgeYqytqean |
| 1177-1191 | yncsqdeYfdheeqv |

>PLOC00004 PS00004 CAMP_PHOSPHO_SITE cAMP- and cGMP-dependent protein kinase
phosphorylation site [pattern] [Warning: pattern with a high probability of
occurrence].

| | |
|---|---|
| 1058-1061 | RKcS |

>PDOC00005 PS00005 PKC_PHOSPHO_SITE Protein kinase C phosphorylation site
[pattern] [Warning: pattern with a high probability of occurrence].

| | |
|---|---|
| 74-76 | TcK |
| 114-116 | SvK |
| 138-140 | SvR |
| 377-379 | TcR |
| 388-390 | TeR |
| 560-562 | SwR |
| 643-645 | SlR |
| 684-686 | SdR |
| 768-770 | TfK |
| 906-908 | TfK |
| 1029-1031 | SwK |
| 1260-1262 | SsK |
| 1290-1292 | TlR |
| 1304-1306 | TtR |
| 1323-1325 | TtR |
| 1488-1490 | TlK |
| 1517-1519 | TnK |
| 1550-1552 | StR |
| 1565-1567 | SsR |
| 1657-1659 | TiK |
| 1686-1688 | TaK |
| 1714-1716 | TpK |
| 1734-1736 | SsR |
| 1835-1837 | TsR |
| 1847-1849 | TaK |
| 1895-1897 | SsR |
| 1908-1910 | TyR |
| 2086-2088 | TpR |
| 2169-2171 | SvR |
| 2178-2180 | TfK |

>PDOC00006 PS00006 CK2_PHOSPHO_SITE Casein kinase U phosphorylation site
[pattern] [Warning: pattern with a high probability of occurrence].

| | |
|---|---|
| 38-41 | TapD |
| 54-57 | StfD |
| 74-77 | TckD |
| 107-110 | TvsE |
| 114-117 | SvkD |
| 214-217 | TfqD |
| 273-276 | TlsE |
| 319-322 | SnsE |
| 405-408 | TtfD |
| 444-447 | ShsE |
| 457-460 | SrqD |
| 465-468 | SqdE |
| 539-542 | TtdD |
| 599-602 | TvfE |
| 654-657 | SsvD |
| 682-685 | SlsD |
| 800-803 | TkcE |
| 946-949 | TgeE |
| 1022-1025 | SelE |
| 1029-1032 | SwkE |
| 1054-1057 | SwaE |
| 1093-1096 | SggD |
| 1171-1174 | SniE |
| 1180-1183 | SqdE |
| 1266-1269 | SsgE |
| 1346-1349 | TnqE |
| 1383-1386 | TatE |
| 1392-1395 | TttE |
| 1436-1439 | ShpE |
| 1754-1757 | SstD |
| 1766-1769 | TpsD |
| 1908-1911 | TyrE |
| 1936-1939 | TpsD |
| 1997-2000 | TvpD |
| 2070-2073 | SlpE |
| 2089-2092 | SrgE |
| 2096-2099 | TswE |
| 2169-2172 | SvrE |
| 2189-2192 | TrcE |

>PDOC00007 PS00007 TYR_PHOSPHO_SITE Tyrosine kinase phosphorylation site
[pattern] [Warning: pattern with a high probability of occurrence].

| | |
|---|---|
| 2101-2109 | RaagEgraY |

>PDOC00008 PS00008 MYRISTYL N-myristoylation site [pattern] [Warning: pattern
with a high probability of occurrence].

| | |
|---|---|
| 12-17 | GAllSA |
| 18-23 | GLanTS |
| 43-48 | GQcsTW |
| 170-175 | GQmcGL |
| 174-179 | GLcgNF |
| 177-182 | GNfdGK |
| 285-290 | GQpvAL |
| 299-304 | GQcpAN |
| 338-343 | GTdlND |
| 401-406 | GSfvTT |
| 432-437 | GAlmAV |
| 442-447 | GVshSE |
| 526-531 | GQtrGL |
| 530-535 | GLcgNF |
| 533-538 | GNfnGD |
| 548-553 | CIaeGT |
| 705-710 | GTylNQ |
| 805-810 | GCvcAE |
| 811-816 | GLyeNA |
| 899-904 | GVnySQ |
| 920-925 | GVtcSR |
| 955-960 | GVtpGA |
| 999-1004 | GLcgNF |
| 1002-1007 | GNfnGN |
| 1090-1095 | GCdsGG |
| 1175-1180 | GCynCS |
| 1213-1218 | GSrpTQ |
| 1224-1229 | GTstTI |
| 1230-1235 | GLlsST |
| 1284-1289 | GLppTA |
| 1337-1342 | GTspTL |
| 1352-1357 | GTtaTQ |
| 1493-1498 | GSthTA |
| 1507-1512 | GTsqAH |
| 1662-1667 | GSthTA |
| 1676-1681 | GTsqSL |
| 1823-1828 | GSthTA |
| 1884-1889 | GTpvAH |
| 2033-2038 | GSlaCT |
| 2093-2098 | GAgtSW |
| 2181-2186 | GCmaNV |
| 2193-2198 | GAciSA |

>PDOC00009 PS00009 AMIDATION Amidation site [pattern] [Warning: pattern with a
high probability of occurrence].

| | |
|---|---|
| 2235-2238 | pGRR |

>PDOC00012 PS01225 CTCK_2 C-terminal cystine knot domain [profile].

| | |
|---|---|
| 2168-2257 | CSVREQQ-EEITFKGC--MANVTVTRCEGACISAASFNIITQQVDARCSCCRPLHSYEQQ |
| | LELPCPDpstpGRRLVLTLQVFSHCVCSsVACG |

>PDOC00928 PS50184 VWEC_2 VWFC domain [profile].

| | |
|---|---|
| | The following hit is below threshold (may be spurious) |
| 358-418 | --CVLHGAMYAPGEVTIAA-CQTCRCTLGRWVCTERPCP--GHCSLEGGSFvttfdarpy |
| | rFHGTC ----- |

>PDOC50099 PS50311 CYS_RICH Cysteine-rich region [profile].

| | |
|---|---|
| 296-396 | Csvgqcpanqvyqecgsacvktcsnsehscsssctfgcfcpegtdlndlsnnhtcvpvtq |
| | cpcvlhgamyapgevtlaacqtcrctlgrwvcterpcpghC |
| 784-867 | Captcqmlatgvacvptkcepgcvcaeglyenaygqcvppeecpcefsgvsypggaelht |
| | dcrtcscsrgrwacqqgthcpstC |
| | The following hit is below threshold (may be spurious) |
| 1084-1130 | CvrdacgcdsggdceclcdavaayaqacldkgvcvdwrtpafcpiyC |

>PDOC50099 PS50316 HIS_RICH Histidine-rich region [profile].

| | |
|---|---|
| | The following hit is below threshold (may be spurious) |
| 1928-2009 | Hhylsnpitpsdhtshsrstflhlfsdskyshshhpypctdvhfcldplnanshqpyhqa |
| | pwshlvayhtvpdqlphcpwkH |

>PDOC50099 PS50099 PRO_RICH Proline-rich region [profile].

| | |
|---|---|
| 1194-1448 | Pcmppttpqppttpqlpttgsrptqvwpmtgtsttigllsstgpspssnhtpasptqtpl |
| | lpatltsskptassgepprpttavtpqatsglpptatlrstatkptvtqattratastas |
| | pattstaqsttrttmtlptpatsgtsptlpkstnqelpgttatqttgprptpasttgptt |
| | pqpgqptrptatettqtrttteyttpqtphtthspptagspvpstgpvtatsfhatttyp |
| | tpshpettlpthvpP |

>PDOC50099 PS50325 THR_RICH Threonine-rich region [profile].

| | |
|---|---|
| 1199-1908 | Ttpqppttpqlpttgsrptqvwpmtgtsttigllsstgpspssnhtpasptqtpllpatl |
| | tsskptassgepprpttavtpqatsglpptatlrstatkptvtqattratastaspatts |
| | taqsttrttmtlptpatsgtsptlpkstnqelpgttatqttgprptpasttgpttpqpgq |
| | ptrptatettqtrttteyttpqtphtthspptagspvpstgpvtatsfhatttyptpshp |
| | ettlpthvppfstslvtpsthtvitpthaqmassasnhsaptgtipppttlkatgsthta |
| | ppitpttsgtsqahssfstnktptslhshtssthhpevtptsttsitpnptstrtrtpma |
| | htnsatssrpptpftthspptgsspisstgpmtapsfhatttypt pshpqttlpthvpsf |
| | stslvtpsthivitpthaqmatsasihsmqtgtipppttikatgsthtappmtpttsgts |
| | qslssfstaktstslpyhtssthhpevtptsttnitpkhtstgtrtpvahttsatssrlp |
| | tpftthspptgsspisstdhhylsnpitpsdhtshsrstflhllgdskysqghhpypctd |
| | ghfclhplnanrapflplttlmntgsthtaplitvttsrtsqvhssfstaktstsllsha |
| | ssthhpeittnstttitpnptstgtgtpvahttsatssrltttlhhtlpT |

Prosite Results for SCS0005:
>PDOC00001 PS00001 ASN_GLYCOSYLATION N-glycosylation site [pattern] [Warning:
pattern with a high probability of occurrence].

| | |
|---|---|
| 205-208 | NKTC |
| 258-261 | NCST |
| 415-418 | NCTC |
| 524-527 | NVTI |
| 1369-1372 | NCSE |
| 1442-1445 | NCTY |
| 1562-1565 | NNTE |
| 1598-1601 | NVST |
| 1741-1744 | NDSA |
| 1852-1855 | NTSR |
| 1882-1885 | NCSN |
| 1891-1894 | NGTL |
| 1960-1963 | NTSK |
| 2101-2104 | NQST |
| 2164-2167 | NVTL |

>PDOC00003 PS00003 SULFATION Tyrosine sulfation site [rule] [Warning: rule
with a high probability of occurrence].

| | |
|---|---|
| 2172-2186 | gssrafsYteveecg |

>PDOC00004 PS00004 CAMP_PHOSPHO_SITE cAMP- and cGMP-dependent protein kinase
phosphorylation site [pattern] [Warning: pattern with a high probability of
occurrence].

| | |
|---|---|
| 846-849 | KKtS |

>PDOC00005 PS00005 PKC_PHOSPHO_SITE Protein kinase C phosphorylation site
[pattern] [Warning: pattern with a high probability of occurrence].

| | |
|---|---|
| 35-37 | SyK |
| 62-64 | SlR |
| 599-601 | TfK |
| 701-703 | SyR |
| 707-709 | TiR |
| 773-775 | SfR |
| 824-826 | TiR |
| 894-896 | SwK |
| 984-986 | SwR |
| 1018-1020 | TcR |
| 1227-1229 | TpR |
| 1382-1384 | SlR |
| 1491-1493 | TrK |
| 1581-1583 | TpR |
| 1814-1816 | TcR |
| 1853-1855 | TsR |
| 1908-1910 | TcR |
| 1961-1963 | TsK |
| 1998-2000 | TtK |
| 1999-2001 | TkK |
| 2024-2026 | TpR |
| 2161-2163 | SlR |
| 2173-2175 | SsR |

>PDOC00006 PS00006 CK2_PHOSPHO_SITE Casein kinase TT phosphorylation site
[pattern] [Warning: pattern with a high probability of occurrence].

| | |
|---|---|
| 177-180 | TkVE |
| 231-234 | TpmE |
| 286-289 | SylE |
| 310-313 | TlaE |
| 356-359 | SnqE |
| 378-381 | TvlD |
| 424-427 | ScqE |
| 443-446 | StfD |
| 481-484 | TdsE |
| 612-615 | SfeD |
| 638-641 | TpgD |
| 689-692 | TaeD |
| 769-772 | StqD |
| 900-903 | ScpD |
| 958-961 | SggD |
| 1180-1183 | ShpE |
| 1196-1199 | SreE |
| 1342-1345 | StsE |
| 1438-1441 | TflD |
| 1536-1539 | TipE |
| 1589-1592 | ScsE |
| 1600-1603 | SipD |
| 1684-1687 | TdlD |
| 1691-1694 | SslE |
| 1871-1874 | TyqE |
| 1904-1907 | SlcE |
| 1974-1977 | TwsD |
| 2048-2051 | StpE |
| 2076-2079 | SaqD |
| 2120-2123 | SssE |
| 2178-2181 | SytE |
| 2180-2183 | TevE |

>PDOC00008 PS00008 MYRISTYL N-myristoylation site [pattern] [Warning: pattern
with a high probability of occurrence].

| | |
|---|---|
| 30-35 | GSseSS |
| 272-277 | GQlfSG |
| 384-389 | GQtgCV |
| 406-411 | GAtyST |
| 420-425 | GGrwSC |
| 439-444 | GAhfST |
| 479-484 | GLtdSE |
| 542-547 | GLgiNL |
| 565-570 | GQtcGL |
| 569-574 | GLcgNF |
| 572-577 | GNfnST |
| 587-592 | GVveAT |
| 645-650 | GCqkSC |
| 685-690 | GGciTA |
| 686-691 | GCitAE |
| 711-716 | GCntCT |
| 746-751 | GQsySF |
| 765-770 | GGkdST |
| 784-789 | GTtgTT |
| 787-792 | GTtcSK |
| 814-819 | GTdeSQ |
| 864-869 | GLcgNF |
| 980-985 | GLcvSW |
| 1032-1037 | GLeaST |
| 1230-1235 | GCpvTS |
| 1240-1245 | GTspTN |
| 1351-1356 | GCpnAV |
| 1434-1439 | GTyyTF |
| 1468-1473 | GAedGL |
| 1497-1502 | GVmtNE |
| 1523-1528 | GIvvSR |
| 1548-1553 | GLifSV |
| 1566-1571 | GQcgTC |
| 1569-1574 | GTctND |
| 1584-1589 | GTvvAS |
| 1740-1745 | GNdsAS |
| 1779-1784 | GCprCL |
| 1861-1866 | GCpeGA |
| 1898-1903 | GAvvSS |
| 1915-1920 | GGppSD |
| 1946-1951 | GQccGT |
| 2072-2077 | GAplSA |
| 2118-2123 | GCssSE |
| 2172-2177 | GSsrAF |

>PDOC00009 PS00009 AMIDATION Amidation site [pattern] [Warning: pattern with a
high probability of occurrence].

| | |
|---|---|
| 3-6 | vGRR |
| 2188-2191 | mGRR |

>PDOC00013 PS00013 PROKAR_LIPOPROTEIN Prokaryotic membrane lipoprotein lipid
attachment site [rule].

| | |
|---|---|
| 10-20 | LLWALALALAC |

>PDOC00016 PS00016 RGD Cell attachment sequence [pattern] [Warning: pattern
with a high probability of occurrence].

| | |
|---|---|
| 1023-1075 | RGD |

>PDOC00029 PS00029 LEUCINE_ZIPPER Leucine zipper pattern [pattern] [Warning:
pattern with a high probability of occurrence].

| | |
|---|---|
| 274-295 | LfsgcvaLvdvgsyLeacrqdL |

>PDOC00912 PS01185 CTCK_1 C-terminal cystine knot signature [pattern].

| | |
|---|---|
| 2154-2192 | CCqelrtslrnvtlhCtdGssrafsyteveeCgCmgrrC |

>PDOC00912 PS01225 CTCK_2 C-terminal cystine xnot domain [profile].

| | |
|---|---|
| 2105-2193 | CAVYHRS-LIIQQQGCSSSEPVRLAYCRGNCGDSsSMYSLEGNTVEHRCQCCQELRTSLR |
| | NVTLHCTDGSSRAFSYTEVEECGCMgRRCP |

>PDOC00928 PS01208 VWFC_1 VWFC domain signature [pattern].

| | |
|---|---|
| 1801-1849 | Cqe.CtCeaatwtlt......CrpklCplppa.....CplpgfvpvpaapqagqCCpqysC |
| 1906-1952 | Cet.CrCelpggppsdafvvsCetqiCnth.......Cpvgfeyqeqsgq....CCgt..C |

>PDOC00928 PS50184 VWFC_2 VWFC domain [profile].

| | |
|---|---|
| | The following bit is below threshold (may be spurious) |
| 394-465 | CACVYN.GAAYAPGATYSTD -CTNCTCSG.......GRWS.CQEVPCP --..GTCSVLGG |
| | AHfstfdgkqytVHGDCSYvltkPCD |
| | The following bit is below threshold (may be spurious) |
| 1352-1418 | --CPNA.VPPRKKGETWATPNCSEATCEG.......NNVIsLRPRTCPRVekPTCANGYP |
| | AVkv.......aDQDGCCHh..yQCQ |
| 1781-1850 | PRCLGPhGEPVKVGHT VGMD-CQECTCEAa......TWTLtCRPKLCPLP..PACPLPGF |
| | VPvpa.....apQAGQCCPq..ySCA |
| 1886-1953 | TVCSIN.GTLYQPGAVVSSSLCETCRCELpggppsdAFVVsCETQICN --..THCPVGFE |
| | YQe.........QSGQCCG....TCV |

>PDOC50099 PS50311 CYS_RICH Cysteine-rich region [profile].

| | |
|---|---|
| 291-434 | Crqdlcfcedtdllscvchtlaeysrqcth agglpqdwrgpdfcpqkcpnnmqyhecrsp |
| | cadtcsnqehsracedhcvagcfcpegtvlddigqtgcvpvskcacvyngaayapgatys |
| | tdctnctcsggrwscqevpcpgtC |
| 646-733 | Cqkachtldmtcwcllalqyspqcvcpgcvcpdglvadgeggci taedcpcvhneasyrag |
| | qtirvgcntctcdsrmwrctddpclatC |
| 949-1026 | Cvndacacdsggdcecfctavaayaqachevglcvswrtpsicplfcdyynpegqcewhy |
| | qpcgvpclrtcrnprgdC |
| | The following bit is below threshold (may be spurious) |
| 1411-1421 | CchhyqcqcvC |
| 1801-1957 | Cqectceaatwtltcrpklcplppacplpgfvpvpaapqagqccpqyscacntsrcpapv |
| | gcpegaraiptyqegaccpvqncswtvcsingtlyqpgavvssslcetc rcelpggppsd |
| | afvvscetqicnthcpvgfeyqeqsgqccgtcvqvaC |

>PDOC50099 PS50324 SER_RICH Serine-rich region [profile].

| | |
|---|---|
| 1250-1278 | SlstsmvsasvastsvasssvasssvayS |

>PDOC50099 PS50325 THR_RICH Threonine-rich region [profile].

| | |
|---|---|
| 1037-1118 | Ttsgpgtslspvpttsttsapttsttsgpgttpspvpttsttsapttsttsgpgttpspv |
| | pttsttpvsktstshlsvsktT |
| 1613-1641 | TpttvgpttvgsttvgpttvgsttvgptT |

>PDOC50280 PS50868 POST_SET Post-SET domain [profile].

| | |
|---|---|
| | The following bit is below threshold (may be spurious) |
| 1845-1861 | PQYSCACNTSRCPAPVG |

>PDOC50042 PS50842 EXPANSIN_EG45 Expansin, family-45 endoglucanase-like domain
[profile].

| | |
|---|---|
| | The following bit is below threshold (may be spurious) |
| 568-656 | CGLCGNFNsIQAdDFrtLSGVVEATAAAFFNTFKTQAACPNIRNSfedp........... |
| | .....cslsVENVCAAP...MVFFDCRNATPGdtGAGCQKSCHTLDMT ------------ |
| | ------------------------ |
| | The following bit in below threshold (may be spurious) |
| 1944-2009 | --------.---.--..-------------QSGQCCGTCVQVACVtntskspahlfypge |
| | twsdagn hcVTHQCEKHqdgLVVVTTKKACPP.. -LSCS--------------------- |
| | ------------------------ |

ELM Results for SCS0004 variant:

| | Instances | | | | |
|---|---|---|---|---|---|
| | (Matched | | | Cell | |
| Elm Name | Sequence) | Positions | Elm Description | Compartment | Pattern |
| CLV_NDR_NDR_1 | RRS | 1052-1054 | N-Arg dibasic | extracellular, | .RK\|RR[\hat{ }KR] |
| | ERK | 1057-1059 | convertase | Golgi | |
| | | | (nardilysine) cleavage | apparatus, | |
| | | | site (Xaa-\|-Arg-Lys or | cell surface | |
| | | | Arg-\|-Arg-Xaa) | | |
| CLV_PCSK_PC1ET2_1 | KRC | 615-617 | NEC1/NEC2 cleavage | extracellular, | KR. |
| | | | site (Lys-Arg-\|-Xaa) | Golgi | |
| | | | | apparatus, | |
| | | | | Golgi | |
| | | | | membrane | |
| MOD_GlcNHglycan | GSAC | 311-314 | Glycosaminoglycan | extracellular, | [ED](0, 3).(S)[GA], |
| | DSGG | 1092-1095 | attachment site | Golgi | |
| | SSGE | 1266-1269 | | apparatus | |
| | TSGL | 1282-1285 | | | |
| | TSGT | 1335-1338 | | | |
| | SSAS | 1471-1474 | | | |
| | HSAP | 1476-1479 | | | |
| | TSGT | 1505-1508 | | | |
| | NSAT | 1561-1564 | | | |
| | TSAS | 1640-1643 | | | |
| | TSGT | 1674-1677 | | | |
| | TSAT | 1730-1733 | | | |
| | TSAT | 1891-1894 | | | |
| | GSGQ | 2155-2158 | | | |
| MOD_N-GLC_1 | NTS | 21-23 | Generic motif for N- | extracellular, | (N)[\hat{ }P][ST]\|(N)[\hat{ }P][ST] |
| | | | | | [\hat{ }P] |
| | NSS | 268-270 | glycosylation. Shakin- | Golgi | |
| | NHT | 347-349 | Eshleman et al, | apparatus, | |
| | NCT | 658-660 | showed that Trp, Asp, | endoplasmic | |
| | NCT | 1151-1153 | and Glu are uncommon | reticulum | |
| | NCS | 1178-1180 | before the Ser/Thr | | |
| | NHT | 1242-1244 | position. Efficient | | |
| | NHS | 1475-1477 | glycosylation usually | | |
| | NKT | 1518-1520 | occurs when ˜60 | | |
| | NIT | 1712-1714 | residues or more | | |
| | NST | 1889-1871 | separate the | | |
| | | | glycosylation acceptor | | |
| | | | site from the C- | | |
| | | | terminus | | |
| MOD_PLK | ECSV | 242-245 | Site phosphorylated by | not annotated | [DE].[ST][ILFWMVA] |
| | EGTA | 551-554 | the Polo-like-kinase | | |
| | EETF | 625-628 | | | |
| | DVSF | 1038-1041 | | | |
| | ETTL | 1439-1442 | | | |
| | EQSL | 1911-1914 | | | |
| MOD_CMANNOS | WGHW | 2141-2144 | Motif for attachment of | not annotated | W..W |
| | | | a mannosyl residue to | | |
| | | | a tryptophan | | |
| MOD_OFUCOSY | CINGRLSC | 745-752 | Site for attachment of a | not annotated | C.{3, 5}[ST]C |
| | | | fucose residue to serin | | |
| LIG_SH2_STAT5 | YTSP | 24-27 | STAT5 Src Homology 2 | not annotated | Y[VLTFIC].. |
| | YVHA | 638-641 | (SH2) domain binding | | |
| | YVAS | 1018-1021 | motif. | | |
| | YCGF | 1129-1132 | | | |
| | YTQE | 1146-1149 | | | |
| | YFDH | 1184-1187 | | | |
| | YTTP | 1396-1399 | | | |
| | YLSN | 1760-1763 | | | |
| | YLSN | 1930-1933 | | | |
| | YVPL | 2137-2140 | | | |

ELM Results for SCS0005:

| | Instances | | | | |
|---|---|---|---|---|---|
| | (Matched | | | Cell | |
| Elm Name | Sequence) | Positions | Elm Description | Compartment | Pattern |
| CLV_NDR_NDR_1 | FRK | 916-918 | N-Arg dibasic convertase | extracellular, | .RK\|RR[\hat{ }KR] |
| | RRP | 1166-1168 | (nardilysine) cleavage site | Golgi | |
| | | | (Xaa-\|-Arg-Lys or Arg-\|-Arg- | apparatus, | |
| | | | Xaa) | cell surface | |
| LIG_RGD | RGD | 1023-1025 | This motif can be found in | extracellular, | RGD |
| | | | proteins of the extracellular | integrin | |
| | | | matrix and it is recognized | | |
| | | | by different members of the | | |
| | | | integrin family. The structure | | |
| | | | of the tenth type III module | | |
| | | | of fibronectin has shown | | |
| | | | that the RGD motif lies on a | | |
| | | | flexible loop. | | |
| MOD_GlcNHglycan | PSGV | 49-52 | Glycosaminoglycan | extracellular, | [ED](0, 3).(S)[GA], |
| | FSGC | 275-278 | attachment site | Golgi | |
| | DSGG | 957-960 | | apparatus | |
| | TSGP | 1038-1041 | | | |
| | TSAP | 1054-1057 | | | |
| | TSGP | 1062-1065 | | | |
| | TSAP | 1078-1081 | | | |
| | TSGP | 1088-1089 | | | |
| | RSGE | 1159-1162 | | | |
| | VSAS | 1256-1259 | | | |
| | EMSGL | 1592-1598 | | | |
| | MSGL | 1593-1596 | | | |
| | DSAS | 1742-1745 | | | |
| | TSAQ | 1770-1773 | | | |
| | LSAQ | 2075-2078 | | | |
| | ESGS | 2213-2216 | | | |
| MOD_N-GLC_1 | NCS | 258-260 | Generic motif for N- | extracellular, | (N)[\hat{ }P][ST]\|(N)[\hat{ }P][ST] |
| | | | | | [\hat{ }P] |
| | NVS | 1598-1600 | glycosylation. Shakin- | Golgi | |
| | NDS | 1741-1743 | Eshleman et al, showed that | apparatus, | |
| | NTS | 1852-1854 | Trp, Asp, and Glu are | endoplasmic | |
| | NCS | 1882-1884 | uncommon before the | reticulum | |
| | NTS | 1960-1962 | Ser/Thr position. Efficient | | |
| | NQS | 2101-2103 | glycosylation usually occurs | | |
| | | | when ˜60 residues or more | | |
| | | | separate the glycosylation | | |
| | | | acceptor site from the C- | | |
| | | | terminus | | |
| MOD_PLK | EATA | 590-593 | Site phosphorylated by the | not annotated | [DE].[ST][ILFWMVA] |
| | | | Polo-like-kinase | | |
| vMOD_CMANNOS | WTKW | [symbol] | [symbol] | not annotated | W..W |
| | | | mannosyl residue to a | | |
| | | | tryptophan | | |
| LIG_SH2_STAT5 | YLEA | 287-290 | STAT5 Src Homology 2 | not annotated | Y[VLTFIC].. |
| | YCYG | 1737-1740 | (SH2) domain binding motif. | | |

Description of Domains and Patterns:
von Willebrand factor type D domain: A family of growth regulators (originally called cef10, connective tissue growth factor, fisp-12, cyr61, or, alternatively, A βIG-M1 and βIG-M2), all belong to immediate early genes expressed after induction by growth factors or certain oncogenes. Sequence analysis of this family revealed the presence of four distinct modules. Each module has homologues in other extracellular mosaic proteins such as Von Willebrand factor, slit, thrombospondins, fibrillar collagens, IGF-binding proteins and mucins. Classification and analysis of these modules suggests the location of binding regions and, by analogy to better characterized modules in other proteins, sheds some light onto the structure of this new family MEDLINE:93327926.
The vWF domain is found in various plasma proteins: complement factors B, C2, CR3 and CR4; the integrins (I-domains); collagen types VI, VII, XII and XIV; and other extracellular proteins MEDLINE:94018965, MEDLINE:94194513 MEDLINE:91323531. Although the majority of VWA-containing proteins are extracellular, the most ancient ones present in all eukaryotes are all intracellular proteins involved in functions such as transcription, DNA repair, ribosomal and membrane transport and the proteasome. A common feature appears to be involvement in multiprotein complexes. Proteins that incorporate vWF domains participate in numerous biological events (e.g. cell adhesion, migration, homing, pattern formation, and signal transduction), involving interaction with a large array of ligands MEDLINE:94018965. A number of human diseases arise from mutations in VWA domains. Secondary structure prediction from 75 aligned vWF sequences has revealed a largely alternating sequence of α-helices and β-strands MEDLINE:94194513.
One of the functions of von Willebrand factor (vWF) is to serve as a carrier of dotting factor VII (FVIII). The native conformation of the D′ domain of vWF is not only required for factor VII (FVIII) binding but also for normal multimerization and optimal secretion MEDLINE:20269787.
Trypsin inhibitor like cysteine rich domain: This domain is found in trypsin inhibitors as well as in many extracellular proteins. The domain typically contains ten cysteine residues that form five disulphide bonds. The cysteine residues that form the disulphide bonds are 1-7, 2-6, 3-5, 4-10 and 8-9.
von Willebrand factor type C domain: The vWF domain is found in various plasma proteins:complement factors B, C2, CR3 and CR4; the integrins (I-domains); collagen types VI, VII, XII and XIV; and other extracellular proteins MEDLINE:94018965, MEDLINE:94194513, MEDLINE:91323531. Although the majority of VWA-containing proteins are extracellular, the most ancient ones present in all eukaryotes are all intracellular proteins involved in functions such as transcription, DNA repair, ribosomal and membrane transport and the proteasome. A common feature appears to be involvement in multiprotein complexes. Proteins that incorporate vWF domains participate in numerous biological events (e.g. cell adhesion, migration, homing, pattern formation, and signal transduction), involving interaction with a large array of ligands MEDLINE:94018965. A number of human diseases arise from mutations in VWA domains. Secondary structure prediction from 75 aligned vWF sequences has revealed a largely alternating sequence of α-helices and β-strands MEDLINE:94194513. The domain is named after the von Willebrand factor (VWF) type C repeat which is found in multidomain protein/multifunctional proteins involved in maintaining homeostasis MEDLINE:87213283, MEDLINE:91323531. For the von Willebrand factor the duplicated VWFC domain is thought to participate in oligomerization, but not in the initial dimerization step MEDLINE:91177957. The presence of this region in a number of other complex-forming proteins points to the possible involvement of the VWFC domain in complex formation.
WAP-type (Whey Acidic Protein) ‘four-disulfide core’: A group of proteins containing 8 characteristically-spaced cysteine residues, which are involved in disulphide bond formation, have been termed ‘4-disulphide core’ proteins MEDLINE:82196900. While the pattern of conserved cysteines suggests that the sequences may adopt a similar fold, the overall degree of sequence similarity is low (e.g. a few Pro and Glyresidues are reasonably well conserved, as is the polar/acidic nature of residues between the third and fourth Cys, but otherwise there is little sequence conservation). The group of sequences that share this pattern include whey acidic protein (WAP) MEDLINE:82196900; elafin (an elastase-specific inhibitor from human skin) MEDLINE:90368643; WDNM1 protein (which is involved in the metastatic potential of adenocarcinomas in rats MEDLINE:88310901; Kallmann syndrome protein MEDLINE:92005720; and caltrin-like protein II from guinea pig MEDLINE:90216715 (which inhibits calcium transport into spermatozoa).
NF-X1 type zinc finger: This domain is presumed to be a zinc binding domain. The following pattern describes the zinc finger: C—X(1-6)-H—X—C—X3-C(H/C)—X(3-4)-(H/C)—X(1-10)-C, where X can be any amino acid, and numbers in brackets indicate the number of residues. The two position can be either his or cys. This domain is found in the human transcriptional repressor NK-X1, a repressor of HLA-DRA transcription; the Drosophila shuttle craft protein, which plays an essential role during the late stages of embryonic neurogenesis; and a yeast hypothetical protein YNL023C.
Cystine-knot domain: This domain is found at the C-terminal of glycoprotein hormones and various extracellular proteins. It is believed to be involved in disulphide inked dimerisation.
PCSK cleavage site (NEC1/NEC2 cleavage site): The members of this family are proprotein convertases that process latent precursor proteins into their biologically active products. The prohormone-processing yeast KEX2 protease can act as an intracellular membrane protein or a soluble, secreted endopeptidase. The protein is required for processing of precursors of alpha-factor and killer toxin. PCSK1 (proprotein convertase 1, NEC1) and PCSK2 (proprotein convertase 2, NEC2) are type I proinsulin-processing enzymes that play a key role in regulating insulin biosynthesis. They are also known to cleave proopiomelanocortin, prorenin, proenkephalin, prodynorphin, prosomatostatin and progastrin. PACE4 (paired basic amino acid cleaving system 4, SPC4) is a calcium-dependent serine endoprotease that can cleave precursor protein at their paired basic amino acid processing sites. Some of its substrates are—transforming growth factor beta related proteins, proalbumin, and von Willebrand factor. Furin (PACE, paired basic amino acid cleaving enzyme, membrane associated receptor protein) is serine endoprotease responsible for processing variety of substrates (proparathyroid hormone, transforming growth factor beta 1 precursor, proalbumin, pro-beta-secretase, membrane type-1 matrix metalloproteinase, beta subunit of pro-nerve growth factor and von Willebrand factor). PC7 (proprotein convertase subtilisin/kexin type 7) is a closely related to PACE and PACE4. This calcium-dependent serine endoprotease is concentrated in the trans-Golgi network, associated with the membranes, and is not secreted. It can process proalbumin. PC7 and furin are also thought to be one of the proteases responsible for the activation of HIV envelope glycoproteins gp160 and gp140.
NDR cleavage site: N-Arg dibasic convertase is a metalloendopeptidase primarily cloned from rat brain cortex and tests that cleaves peptide substrates on the N terminus of Arg residues in dibasic stretches. It hydrolyses polypeptides, preferably at -Xaa-+-Arg-Lys-, and less commonly at -Arg-+-Arg-Xaa-, in which Xaa is not Arg or Lys. It is proved that it can cleave alpha-neoendorphin, ANF, dynorphin, preproneurotensin and somatostatin. Also there is an evidence for extracellular localization of active NDR.
SH2 ligand: Src Homology 2 (SH2) domains are small modular domains found within a great number of proteins involved in different signaling pathways. They are able to bind specific motifs containing a phopshorylated tyrosine residue, propagating the signal downstream promoting protein-protein interaction and/or modifying enzymatic activities. Different families of SH2 domains may have different binding specifity, which is usually determined by few residues C-terminal with respect to the pY (positions +1, +2 and +3. Non-phosphorylated peptides do not bind to the SH2 domains. At least three different binding motifs are known: pYEEI (Src-family SH2 domains), pY[IV].[VILP] (SH-PTP2, phospholipase C-gamma), pY.[EN] (GRB2). The interaction between SH2 domains and their substrates is however dependent also to cooperative contacts of other surface regions.
C-Mannosylation site: C-Mannosylation is a type of protein glycosylation, which involves covalent attachment of an alpha-mannopyranosyl residue to the indole C2 carbon atom of tryptophan via a C—C link (Hofsteenge et al., 1994; de Beer et al., 1995). The exact recognition sequence was determined by site-directed mutagenesis of individual amino acids and was found to be WXXW, where the first tryptophan residue becomes C-mannosylated. The significance of the amino acids in both X positions is currently studied. [the shortest peptides used consisted of only four amino acids forming a recognition sequence, WAKW (Hartmann, 2000)] The search for the pattern, restricted to the mammalian proteins that cross the endoplasmic reticulum (ER) membrane, yielded 336 proteins. Some of the proteins found in the database search have already been examined for the presence of C-mannosylation. In total, 49 C-mannosylated tryptophan residues were found in 11 proteins. The precursor in the biosynthesis of (C2-Man)-Trp is dolichylphosphate mannose (Dol-P-Man) precursor in the biosynthetic pathway of C-mannosyltryptophan (Doucey et al., 1998). The whole biosynthetic pathway, from GDPMan, through Dol-P-Man to the C-mannosylated peptide, was reconstructed in vitro. The activity was found in Caenorhabditis elegans, amphibians, birds, mammals, but not in Escherichia coli, insects and yeast (Doucey et al., 1998; Krieg et al., 1997; Hatmann, unpublished results). C-mannosyltransferase activity can be found in most of the parts of the mammalian organism (Doucey, 1998)
O-Fucosylation site: O-Fucose modifications have been described in several different protein contexts including epidermal growth factor-like repeats (important players in several signal transduction systems) and thrombospondin type 1 repeats (in a region involved in cell adhesion). In Notch, a cell-surface signaling receptor required for many developmental events, the O-fucose moieties serve as a substrate for the activity of Fringe, a known modifier of Notch function.
N-glycosylation site: N-glycosylation is the most common modification of secretory and membrane-bound proteins in eukaryotic cells. The whole process of N-glycosylation comprises more than 100 enzymes and transport proteins. The biosynthesis of all N-linked oligosaccharides begins in the ER with a large precursor oligosaccharid. The structure of this oligosaccharide [(Glc)3(Man)9(GlcNAc)2] is the same in plants, animals, and single cell eukaryotes. This precursor is linked to a dolichol, a long-chain polyisoprenoid lipid that act as a carrier for the oligosaccharide. The oligosaccharide then is transfer by an ER enzyme from the dodichol carrier to an asparagine residue on a nascent protein. The oligosaccharide chain is then processed as the glycoprotein moves through the Golgi apparatus. In some cases this modification involves attachment of more mannose groups: in other cases a more complex type of structure is attached.
Glycosaminoglycan attachment site: Proteoglycans are found at the cell surface and in the extracellular matrix. They are important for cell communication, playing a role for example in morphogenesis and development. Mutations in some proteoglycans are associated with an inherited predisposition to cancer. The core protein is modified by attachment of the glycosaminoglycan chain at an exposed serine residue. For heparan sulphate, the process begins by transfer of xylose from UDP-xylose to the serine hydroxyl group by protein xylosyl transferase (EC 2.4.2.26) in the Golgi stack. The system appears to have evolved in metazoan animals.
Integrin binding site: Integrin are the major metazoan receptors. They are heterodimers of alpha and beta subunits that contain a large extracellular domain responsible for ligand binding, a single transmembrane domain and a cytoplasmic domain of 20-70 amino acid residues. Integrin play central role in cell adhesion, cell migration and control of cell differentiation, proliferation and programmed cell death. A hallmark of the integrins is the ability of individual family members to recognize multiple ligands. Most integrins recognize relatively short peptide motif and, in general, a key constituent residue is an acidic amino acid. The ligand specificities rely on both subunits of a given alpha-beta heterodimer. Proteins that contain Arg-Gly-Asp (RGD) attachment site together with the integrins that servers as a receptor for them, constitute a major recognition system for cell adhesion. RGD was originally identified as the sequence in fibronectin that engages the fibronectin receptor, integrin alpha 5 beta 1. RGD sequences have also been found to be responsible for the cell adhesive properties of a number of other proteins, including fibrinogen, von Willebrand factor, and fibronectin.
Leucine zipper pattern: A structure, referred to as the ‘leucine zipper’, has been proposed to explain how some eukaryotic gene regulatory proteins work. The leucine zipper consists of a periodic repetition of leucine residues at every seventh position over a distance covering eight helical turns. The segments containing these periodic arrays of leucine residues seem to exist in an alpha-helical conformation. The leucine side chains extending from one alpha-helix interact with those from a similar alpha helix of a second polypeptide, facilitating dimerization; the structure formed by cooperation of these two regions forms a coiled coil. The leucine zipper pattern is present in many gene regulatory proteins, such as:
   The CCATT-box and enhancer binding protein (C/EBP).
   The cAMP response element (CRE) binding proteins (CREB, CRE-BP1, ATFs).
   The Jun/AP1 family of transcription factors.
   The yeast general control protein GCN4.
   The fos oncogene, and the fos-related proteins fra-1 and fos B.
   The C-myc, L-myc and N-myc oncogenes.
   The octamer-binding transcription factor 2 (Oct-2/OTF-2).
Amidation site: The precursor of hormones and other active peptides which are C-terminally amidated is always directly followed by a glycine residue which provides the amide group, and most often by at least two consecutive basic residues (Arg or Lys) which generally function as an active peptide precursor cleavage site. Although all amino acids can be amidated, neutral hydrophobic residues such as Val or Phe are good substrates, while charged residues such as Asp or Arg are much less reactive. C-terminal amidation has not yet been shown to occur in unicellular organisms or in plants.
N-myristoylation site: An appreciable number of eukaryotic proteins are acylated by the covalent addition of myristate (a C1-4-saturated fatty acid) to their N-terminal residue via an amide linkage. The sequence specificity of the enzyme responsible for this modification, myristoyl CoA:protein N-myristoyl transferase (NMT), has been derived from the sequence of known N-myristoylated proteins and from studies using synthetic peptides. It seems to be the following:
   The N-terminal residue must be glycine.
   In position 2, uncharged residues are allowed. Charged residues, proline and large hydrophobic residues are not allowed.
   In positions 3 and 4, most, if not all, residues are allowed.
   In position 5, small uncharged residues are allowed (Ala, Ser, Thr, Cys, Asn and Gly). Serine is favored.
   In position 6, proline is not allowed.

### REFERENCES

Andersen D C and Krummen L, Curr. Opin. Biotechnol., 13:117-23, 2002.
Baker K N et al., Trends Biotechnol, 20: 149-56, 2002.
Blagoev B and Pandey A, Trends Biochem Sci., 26: 639-41, 2001.
Bock A, Science, 292: 453-4, 2001.
Bunz F, Curr. Opin. Oncol., 14: 73-8, 2002.
Burgess R R and Thompson N B, Curr. Opin. Biotcchnol., 12:450-4, 2001.
Chambers S P, Drug Disc. Today, 14: 759-765, 2002.
Chu L and Robinson D K, Curr. Opin. Biotechnol., 13: 304-8, 2001.
Cleland J L et al., Curr. Opin. Biotechnol., 12: 212-9, 2001.
Coleman R A et al., Drug Discov. Today, 6:1116-1126, 2001.
Constans A, The Scientist, 16(4): 37, 2002.
Davis B G and Robinson M A, Curr. Opin. Drug Discov. Devel., 5: 279-88, 2002.
Dougherty D A, Curr. Opin. Chem. Biol., 4: 645-52, 2000.
Garnett M C, Adv. Drug. Deliv. Rev., 53:171-216, 2001.
Gavilondo J V and Larrick J W, Biotechniques, 29:128-136, 2000.
Gendel S M, Ann. NY Acad. Sci., 964: 87-98, 2002.
Giddings G, Curr. Opin. Biotcchnol., 12: 450-4, 2001.
Golebiowski A et al., Curr. Opin. Drug Discov. Devel., 4:428-34, 2001.
Gupta P et al., Drug Discov. Today, 7: 569-579, 2002.
Haupt K, Nat Biotechnol., 20: 884-885, 2002.
Hruby V J and Balse P M, Curr. Med. Chem, 7: 945-70, 2000.
Johnson D E and Wolfgang G H, Drug Discov. Today, 5: 445-454, 2000.
Kane J F, Curr. Opin. Biotechnol, 6: 494-500, 1995.
Kolb A F, Cloning Stem Cells, 4:65-80, 2002.
Kuroiwa Y et al., Nat Biotechnol, 20: 889-94, 2002.
Lin Cereghino G P et al., Curr. Opin. Biotechnol, 13:329-332, 2001.
Lowe C R et al., J. Biochem. Biophys. Methods, 49:561-74, 2001.
Luo B and Prestwich G D, Exp. Opin. Ther. Patents, 11:1395-1410, 2001.
Mulder N J and Apweiler R. Genome Biol., 3(1):REVIEWS2001, 2002
Nilsson J et al., Protein Expr. Purif., 11: 1-16, 1997.
Pearson W R and Miller W, Methods Enzymol., 210: 575-601, 1992.
Pellois J P et al., Nat Biotcchnol, 20: 922-6, 2002.
Pillai O and Panchagnula R, Curr. Opin. Chem. Biol., 5: 447-451, 2001
Rehm B H, Appl. Microbiol. Biotechnol., 57: 579-92, 2001.
Robinson C R, Nat Biotechnol., 20:879-880, 2002.
Rogov S T and Nekrasov A N, Protein Eng., 14: 459-463, 2001.
Schellekens H, Nat Rev. Drug Discov., 1: 457-62, 2002
Sheibani N, Prep. Biochem. Biotechnol., 29: 77-90, 1999.
Stevanovic S, Nat Rev. Cancer, 2: 514-20, 2002.
Templin M F et al., Trends Biotechnol., 20:160-6, 2002.
Tribbick G, J. Immunol. Methods, 267: 27-35, 2002.
van den Burg B and Eijsink V, Curr. Opin. Biotcchnol., 13: 333-337, 2002.
van Dijk M A and van de Winkel S G, Curr. Opin. Chem. Biol., 5: 368-74, 2001.
Villain M et al., Chem. Biol., 8: 673-9, 2001.

1. An isolated polypeptide having mucin-like activity selected from the group consisting of:
(a) the amino acid sequence as recited in SEQ ID NO: 2, SEQ ID NO: 3 or SEQ ID NO: 7;
(b) the mature form of the polypeptide whose sequence is recited in (a);
(c) an amino acid sequence at least 85% identical to the amino acid sequence recited in (a);
(d) a fusion protein of a polypeptide of (a), (b) or (c); and
(e) a salt of (a), (b), (c) or (d).

2-5. (canceled)

6. A fusion protein of claim 1 wherein the protein comprises a histidine tag.

7. The fusion protein of claim 1 wherein the protein comprises one or more amino acid sequences from a protein selected from the group consisting of: a membrane-bound protein, immunoglobulin constant region, multimerization domains, extracellular proteins, signal peptide-containing proteins, and export signal-containing proteins.

8. (canceled)

9. An antibody which binds specifically to a polypeptide of claim 1.

10. (canceled)

11. The antibody of claim 9 wherein the antibody is a monoclonal antibody, a polyclonal antibody, a humanized antibody, or an antigen binding fragment.

12. A derivative of the polypeptide of claim 1, wherein the polypeptide is derivatized with a radioactive label, fluorescent label, biotin, or cytotoxic agent.

13. (canceled)

14. An isolated nucleic acid comprising a sequence selected from the group consisting of:
(a) a sequence encoding the polypeptide of claim 1;
(b) the sequence set forth in SEQ ID NO: 1 or SEQ ID NO: 6;
(c) a sequence comprising at least 30 consecutive nucleotides that is at least about 85% identical to (b), provided that the sequence encodes a polypeptide with mucin-like activity;
(d) a sequence that hybridizes to (b) under high stringency conditions; and
(e) the complement of (b).

15-16. (canceled)

17. A vector comprising a nucleic acid of claim 14.

18. The vector of claim 17, wherein said nucleic acid is operatively linked to expression control sequences.

19-20. (canceled)

21. A host cell comprising a vector of claim 18.

22. A transgenic animal cell comprising a vector of claim 18.

23. A transgenic non-human animal comprising the vector of claim 18.

24. A method for making a mucin-like polypeptide comprising:
(a) culturing a cell of claim 21 under conditions in which the nucleic acid is expressed, and
(b) recovering the polypeptide encoded by said nucleic acid.

25-28. (canceled)

29. A method of treating or preventing a disease associated with a need for an increase in mucin-like activity comprising administering to a patient in need thereof a polypeptide of claim 1.

30-37. (canceled)

38. A method for identifying a candidate compound as an antagonist/inhibitor or agonist/activator of a mucin-like polypeptide comprising:
(a) contacting the polypeptide of claim 1, said compound, and a mammalian cell or a mammalian cell membrane capable of binding the polypeptide; and
(b) determining whether the compound blocks or enhances the interaction of the polypeptide with the mammalian cell or the mammalian cell membrane, whereby an antagonist/inhibitor or agonist/activator is identified by blocking or enhancing the interaction of the polypeptide with the mammalian cell or the mammalian cell membrane.

39. A method for detecting a mucin-like polypeptide comprising:
(a) providing a protein-containing sample;
(b) contacting said sample with the antibody of claim 9; and
(c) determining the binding of said antibody to a polypeptide of the sample, thereby detecting a mucin-like polypeptide.

40. A method for detecting a nucleic acid encoding a mucin-like polypeptide comprising:
(a) providing a nucleic acids-containing sample;
(b) contacting said sample with a nucleic acid of claim 14; and
(c) determining the hybridization of said nucleic acid with a nucleic acid of the sample, thereby detecting a nucleic acid encoding a mucin-like polypeptide.

41. The method of claim 40 wherein hybridization is determined by Polymerase Chain Reaction

42. A kit for detecting a mucin-like polypeptide comprising a polypeptide of claim 1 and an antibody thereto.
